(19) United States
(12) Patent Application Publication
(10) Pub. No.: US 2014/0348966 A1
(43) Pub. Date: Nov. 27, 2014

(54) GARCINIA BUCHANANII BAKER COMPOUNDS, COMPOSITIONS AND RELATED METHODS

(71) Applicants: Onesmo B. Balemba, Moscow, ID (US); Thomas Hofmann, Freising (DE); Sofie Pasilis, Moscow, ID (US); Gary M. Mawe, Burlington, VT (US); Stuart M. Brierley, Adelaide (AU); Gemma Mazzuoli, Freising (DE); Timo Stark, Freising (DE); Michael Schemann, Freising (DE); Dagmar Kruger, Freising (DE)

(72) Inventors: Onesmo B. Balemba, Moscow, ID (US); Thomas Hofmann, Freising (DE); Sofie Pasilis, Moscow, ID (US); Gary M. Mawe, Burlington, VT (US); Stuart M. Brierley, Adelaide (AU); Gemma Mazzuoli, Freising (DE); Timo Stark, Freising (DE); Michael Schemann, Freising (DE); Dagmar Kruger, Freising (DE)

(21) Appl. No.: 14/367,736

(22) PCT Filed: Dec. 21, 2012
(86) PCT No.: PCT/US12/71441

§ 371 Date: Jun. 20, 2014

Related U.S. Application Data
(60) Provisional application No. 61/579,329, filed on Dec. 22, 2011.

(51) Int. Cl. A61K 36/38; A61K 31/7048; A61K 31/353; C07D 311/32; C07H 17/07

(52) U.S. Cl. CPC A61K 36/38; C07D 311/32; C07H 17/07; A61K 31/353; A61K 31/7048
USPC 424/769; 549/403; 536/18.1

(57) ABSTRACT

Embodiments of the present disclosure related to compounds and compositions derived from G. buchananii baker and methods thereof.

### FIELD OF THE DISCLOSURE

Embodiments of the present disclosure related to compounds and compositions derived from G. buchananii Baker and methods of making and using those compounds and compositions.

### BACKGROUND OF THE DISCLOSURE

In developing nations diarrheal diseases are a significant cause of debilitation, weakened immunity, susceptibility to infection, stunted growth, morbidities, and mortalities of high-risk groups (children, elderly, and HIV/AIDS patients). Each year ˜2 billion cases of diarrheal diseases occur, resulting in ˜1.5 million deaths of children under the age of five. Diarrheal disease is also a major complication in HIV/AIDS patients. In sub-Saharan Africa, ˜1.5 million HIV/AIDS patients die directly or indirectly due to diarrheal each year. In the developed countries, diarrhea illnesses result in a significant number of mortalities, hospitalizations, and outpatient clinic visits, especially in children under 5 years, irritable bowel syndrome patients, and the elderly. These diseases are a significant financial burden to families, businesses, and governments worldwide.

Despite complex etiology and pathobiology, diarrheal disease is fundamentally caused by agents that induce and increase propulsive motility, hypersecretion, variable degrees of inflammation and pain, and altered immunity of the bowel. Many strategies have been used for the treatment of diarrheal diseases. These include antimotility agents such as opiates and somatostatin analogs, absorbents, antisecretory medicines (ekephalinase inhibitors), vaccinations, antibiotics, and oral rehydration therapy. Unfortunately, financial constraints limit synthetic drug availability in developing nations. Today, only 39% of children with diarrhea in developing countries receive the low-osmolarity fluid replacement kit with zinc, a recommended treatment, to prevent dehydration and reduce mortality. Consequently, there is need for development of new interventions and approaches to treat diarrhea, with efforts being directed toward the manufacture of effective, affordable, and accessible formulations that target both symptoms and underlying causes of these diseases.

Herbal extracts have been used for several millennia to treat diarrheal diseases, and it is estimated that up to 80% of the population in some developing countries is currently dependent on such options. This suggests that herbal remedies have the potential to fill the aforementioned niche. Nevertheless, such remedies continue to be regarded with skepticism due to a lack of objective data regarding their safety, mechanisms of action, and efficacy. Widely used antidiarrheal folk remedies include extracts from blackberry roots and bark, Croton lechleri, Galla chinensis, blueberry leaves and fruit, chamomile leaves, apples, green bananas, wood creosote, and Garcinia plant bark and fruit.

Diarrhea is the second leading cause of malnutrition and death among children under five years old in developing countries, causing 20% of all child deaths and for a total of over 1.7 million deaths each year. In addition, diarrheal diseases cause debilitation, morbidities and mortalities among persons displaced by humanitarian crisis and natural disasters, HIV/AIDS patients, and the elderly. Within developed countries, the episodes of diarrhea are relatively high, especially in children, travelers, deployed military personnel although only approximately 1 out of 6 people seek medical treatment.

Diarrhea is a protective response of the gastrointestinal (GI) tract that manifests itself with excessive mucosal fluid secretion and increased propulsive motility to remove the insulting agents, such as pathogens, toxins, drugs, or allergens, from the bowel. As such, it is often associated with a net loss of body fluids due to intestinal hyper-secretion, powerful hyper-motility and cramping pain. Therefore, treatments are directed to combat these symptoms, the causative agents and inflammation. Treatment options include the use of low-osmolarity oral rehydration salts (ORS) supplemented with zinc, to counter intestinal fluid loss and, in children, continuous breast feeding to provide nutrients and enhance child immunity. The regimen also includes anti-motility agents, opiates, somatostatin analogs, absorbents, anti-secretory medicines, vaccinations and antibiotics. However, recommended ORS therapy alone does not treat all underlying causes of diarrheal symptoms, or shorten the duration of illness. In developing countries, ˜60% of children with diarrhea are from poor families and do not use ORS or synthetic drugs. Approximately 80% of the population use herbal remedies to treat all forms of diarrheal diseases.

Oxidative stress is a serious condition that leads to chronic metabolic and degenerative diseases. It is caused by endogenous free radicals, generated in the human body as metabolic by-products, or by free radicals from exogenous sources like ultraviolet light, ionizing radiation, chemotherapeutics, environmental toxins and inflammatory cytokines, which attack various substrates in the body evoking irreparable injuries, cell death and necrosis. The targets of these free radicals are all cellular components, e.g. proteins, carbohydrates, nucleic acids and polyunsaturated fatty acids. The destructive power of these free radicals causing oxidative damage is associated with coronary heart diseases, atherosclerosis, aging, cancer and inflammatory conditions.

Natural phytochemicals having antioxidant activity, such as vitamins (ascorbic acid, vitamin E) carotenoid terpenoids (carotenoids), flavonoid polyphenolics (flavonoids), and alkyl sulfide, reduce risks of many degenerative and metabolic diseases. The antioxidative power of phenolic compounds is mainly due to their redox properties, which play an important role in absorbing and neutralizing free radicals, quenching singlet and triplet oxygen or eliminating peroxides. In the last few years, several investigations have shown that the antioxidant activity of a plant extracts is highly correlated with the extract's phenolic content.

There is a need in the art for treatments for diarrhea and compositions that counteract oxidative stress. Examples of such treatments and compounds are provided herein.

Throughout this description, including the foregoing description of related art, any and all publicly available documents described herein, including any and all U.S. patents, are specifically incorporated by reference herein in their entirety. The foregoing description of related art is not intended in any way as an admission that any of the documents described therein, including pending United States patent applications, are prior art to embodiments of the present disclosure. Moreover, the description herein of any disadvantages associated with the described products, methods, and/or apparatus, is not intended to limit the disclosed embodiments. Indeed, embodiments of the present disclosure may include certain features of the described products, methods, and/or apparatus without suffering from their described disadvantages.

### SUMMARY OF THE DISCLOSURE

Botanical extracts have historically played key roles as precursors to modern drugs and as remedies for a host of diseases including diarrhea and dysentery. Scientific data and indigenous knowledge suggest that Garcinia species can be employed as natural remedies for a variety of illnesses, including diarrheal diseases. Stem and root bark extracts of G. buchananii are currently used in Africa to treat diarrhea, dysentery, abdominal pain, and a range of infectious diseases.

The present invention relates to topical cosmetic and pharmaceutical compositions comprising an effective amount of a biologically active phenolic compound-containing extract, or active fraction thereof, obtainable from G. buchananii. Such compositions are useful in reducing propulsive motility in the gut and may be used in the treatment of gastrointestinal dysfunction, including irritable bowel syndrome (IBS) and diarrhea. In a preferred embodiment, the composition contains at least one G. buchananii flavonoid (e.g., C-glycosyl flavonoids) or a derivative thereof.

The biologically active extracts of G. buchananii useful in this invention can be prepared by any means capable of extracting phenolic compounds from the plant material, using standard extraction techniques. Such extractions include, but are not limited to, ethanol, methanol, ethyl acetate, acetone, chloroform and water, or any other solvent and water. The active phenolic fraction can be obtained from any portion of the plant, but preferably the extract is taken from the aerial portions of the plant, including leaves, twigs, stem, or bark, as well as seeds. The G. buchananii phenolics useful in the present invention are preferably flavonoids, tannins and steroids.

The invention also includes a method of making an extract having the properties outlined in the preceding paragraphs, and a method of reducing colon motility using the extract.

The presently preferred method of making the extract includes at least the steps of: preparing a starting extract from the stem and root bark of G. buchananii, this starting extract including charged and polar compounds and the active fraction; concentration of the extract to a smaller volume; and enrichment of the extract for the active fraction and for (poly) phenol and/or flavonoid compounds generally. In a presently preferred embodiment, the method includes the further steps of: removing most of the free monomer and dimer sugars from the extract. Steps following the preparation of a starting extract, are not necessarily performed in the order listed. Techniques are described for accomplishing each of the indicated steps by chromatography or by precipitation and phase extraction steps.

A more specific description of portions of the disclosure is provided below.

The disclosure provides a composition comprising an isolated compound with Formula 11, shown below.

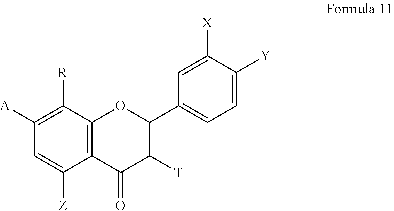

According to certain embodiments of Formula 11, A, X, Y and Z are each independently H, OH, halogen, CN, amine, imine or imide, and R and T are bonds to other molecules with Formula 11. Thus, the compounds of Formula 11 can contain two or more monomers according to Formula 11 attached via positions R and T. In certain embodiments, the bond between two monomers of Formula 11 is between an R bond in the first monomer and a T bond in the second monomer. This is demonstrated in the structure of compound 5 (Formula 5) shown below. However, according to other embodiments a first and second monomer of Formula 11 could be joined at each monomer's S or T positions. Also, the term “halogen” can refer to fluorine (F), chlorine (Cl), bromine (Br), iodine (I), and astatine (At). In other embodiments, the term “halogen” refers to only F, Cl and Br. The disclosure also encompasses stereoisomers for example diastereomers, enantiomers and racemic mixtures of those enantiomers of the compounds described above.

The disclosure provides a composition comprising an isolated compound with Formula 12, shown below.

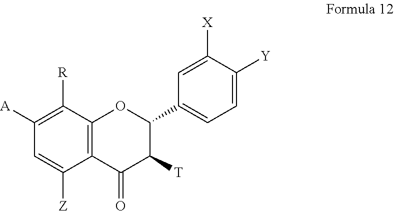

According to certain embodiments of Formula 12, A, X, Y and Z are each independently H, OH, halogen, CN, amine, imine or imide, and R and T are bonds to other molecules with Formula 12. Thus, the compounds of Formula 12 can contain two or more monomers according to Formula 12 attached via positions R and T. In certain embodiments, the bond between two monomers of Formula 12 is between an R bond in the first monomer and a T bond in the second monomer. This is demonstrated in the structure of compound 5 (Formula 5) shown below. However, according to other embodiments a first and second monomer of Formula 12 could be joined at each monomer's S or T positions. Also, the term “halogen” can refer to fluorine (F), chlorine (Cl), bromine (Br), iodine (I), and astatine (At). In other embodiments, the term “halogen” refers to only F, Cl and Br. The disclosure also encompasses stereoisomers for example diastereomers, enantiomers and racemic mixtures of those enantiomers of the compounds described above.

The disclosure provides a composition comprising an isolated compound with Formula 13, shown below.

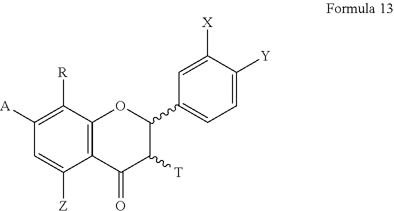

According to certain embodiments of Formula 13, A, X, Y and Z are each independently H, OH, halogen, CN, amine, imine or imide, and R and T are bonds to other molecules with Formula 13. Thus, the compounds of Formula 13 can contain two or more monomers according to Formula 13 attached via positions R and T. In certain embodiments, the bond between two monomers of Formula 13 is between an R bond in the first monomer and a T bond in the second monomer. This is demonstrated in the structure of compound 5 (Formula 5) shown below. However, according to other embodiments a first and second monomer of Formula 13 could be joined at each monomer's S or T positions. Also, the term “halogen” can refer to fluorine (F), chlorine (Cl), bromine (Br), iodine (I), and astatine (At). In other embodiments, the term “halogen” refers to only F, Cl and Br. The disclosure also encompasses stereoisomers for example diastereomers, enantiomers and racemic mixtures of those enantiomers of the compounds described above.

In other embodiments, the isolated compound is a compound with formula 5

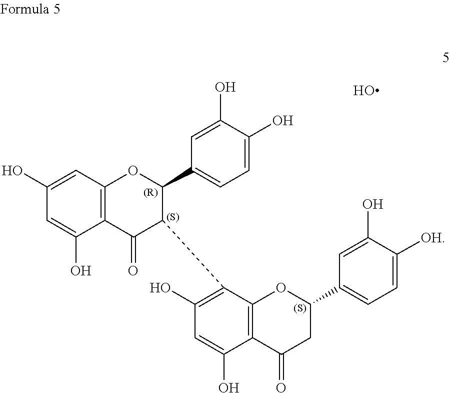

In other embodiments, according to Formula 11 one of A, X, Y or Z must be halogen, CN, amine, imine or imide. In another embodiment, all of A, X, Y and Z are OH.

In other embodiments, the composition described above is a pharmaceutical composition. The pharmaceutical composition can include a pharmaceutically acceptable salt and/or excipient. The pharmaceutical composition can also include a pharmaceutically acceptable binder. The pharmaceutical composition can also be packaged as a pharmaceutically acceptable dosage form. The dosage form can be packaged for any dosage method known in the art. For example, the dosage form can be administered orally. However, in other embodiments, the dosage form is administered rectally, intravenously, subcutaneously, transdermally, transnasally, intraocularly or intraperitoneally. Additional acceptable dosage forms and more specific examples of each are provided below. It is contemplated that the compositions described herein could be administered using any method known in the art or the methods described herein. The disclosure also encompasses stereoisomers for example diastereomers, enantiomers and racemic mixtures of those enantiomers of compounds with the structure of formula 5.

In other embodiments, the pharmaceutical composition includes an isolated compound has a formula that is selected from

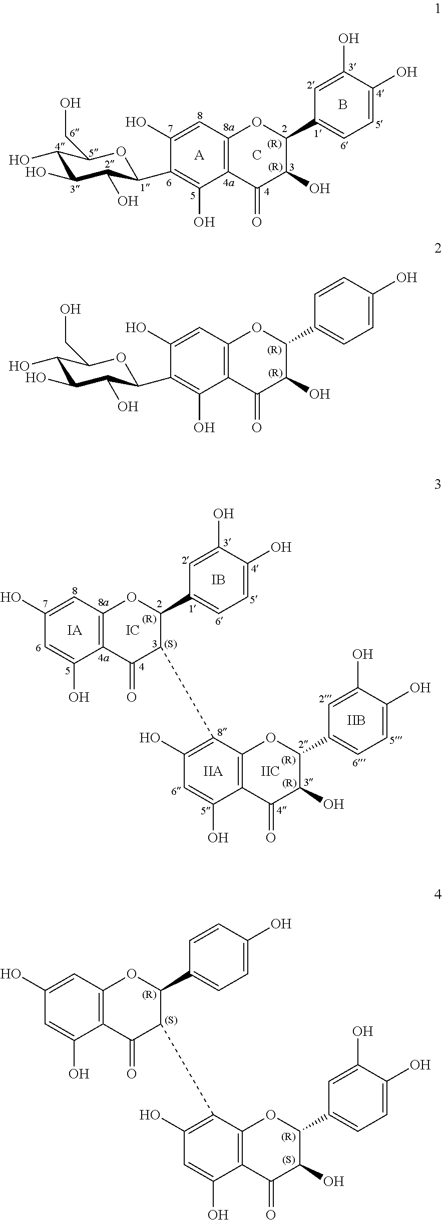

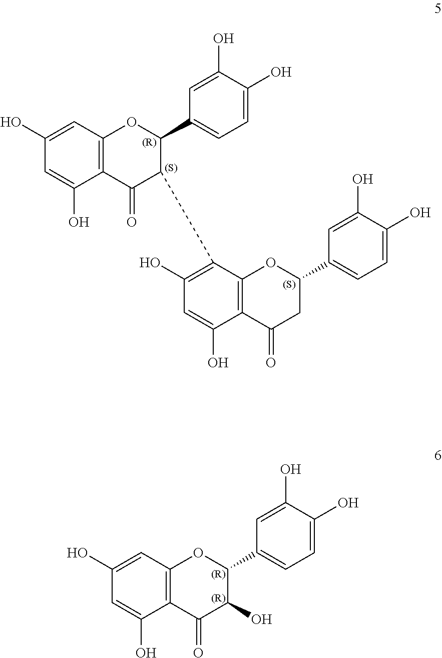

The disclosure also encompasses stereoisomers for example diastereomers, enantiomers and racemic mixtures of those enantiomers of compounds with the structure of formulas 1-6.

The disclosure also provides a composition comprising a fraction of an extract of a component of G. buchananii wherein the extract is enriched in a compound having a formula selected from the group consisting of

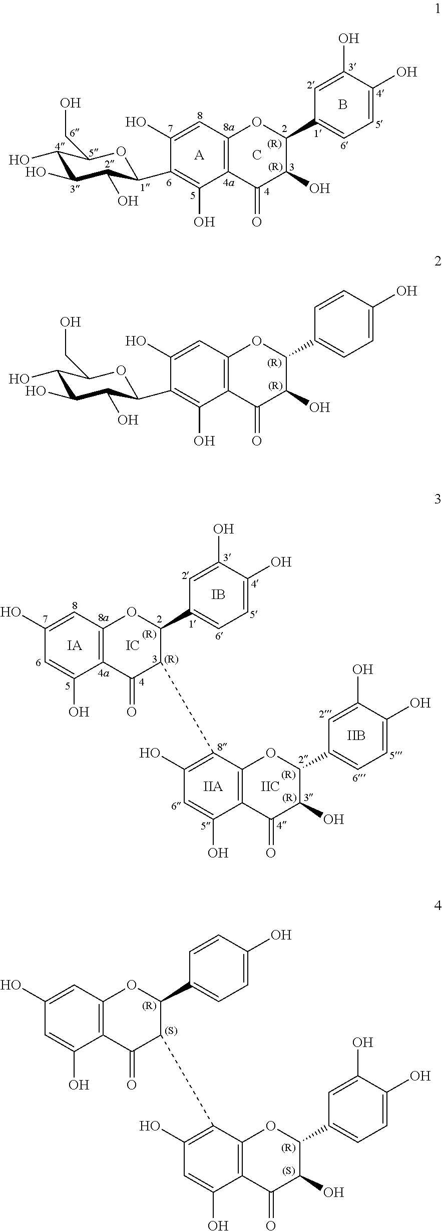

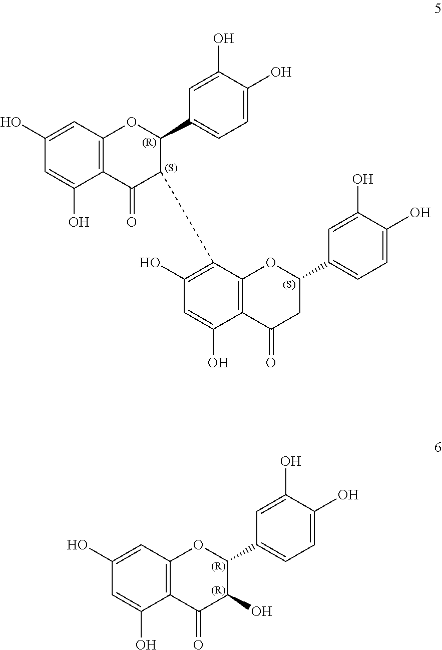

The disclosure also encompasses stereoisomers for example diastereomers, enantiomers and racemic mixtures of those enantiomers of compounds with the structure of formulas 1-6.

In certain embodiments, the component of G. buchananii is the bark of G. buchananii. The bark of G. buchananii can be collected from the stem, root or branches of the plant. In certain embodiments, the bark is collected from the stem of G. buchananii. According to certain embodiments, the fraction is isolated using preparative thin layer chromatography (PTLC). In other embodiments, the fraction is isolated using medium pressure liquid chromatography (MPLC). More detailed methods for isolating these fractions are described below. These or any other methods known in the art can be used to isolate the fractions described herein.

The disclosure also provides a composition comprising a fraction of an extract of a component of G. buchananii wherein the extract is enriched in a compound(s) with antioxidant properties. This can be measured by measuring the antioxidant properties of the fraction. This measurement can then be compared to the antioxidant properties of the whole G. buchananii extract. This enrichment can be a 2-3000% enrichment. In other embodiments, the enrichment is at least 1, 2, 3, 4, 5, 10, 20, 30, 40, 50, 75, 100, 200, 300, 400, 500, 600, 700, 800, 900, 1000, 1500, 2000, 2500 or 3000%. According to certain embodiments, the enrichment of the fraction is compared to the antioxidant properties of the whole G. buchananii extract from which the fraction is derived. According to certain embodiments, the enrichment can be measured using the H₂O₂ assay or the ORAC assay. These assays are described in greater detail below. The measurement can also be made by defining the fraction as having a minimum antioxidant property or a range of antioxidant properties. According to certain embodiments, the fraction has an EC₅₀ of less than 200 using the H₂O₂ assay. According to other embodiments, the fraction has an EC₅₀ of less than 10,000 using the H₂O₂ assay. In other embodiments, the fraction has an EC₅₀ of between 200 and 10,000, between 200 and 6000, 500 and 6000, between 700 and 6000 or between 1000 and 6000 using the H₂O₂ assay. According to other embodiments, the fraction has a value of greater than 1 μmol TE/pmol using the ORAC assay. According to other embodiments, the fraction has value less than than 1300 μmol TE/pmol using the ORAC assay. In other embodiments, the fraction has value of between 5 and 1300, between 5 and 1000, 40 and 1000, between 100 and 1000 or between 140 and 1000 μmol TE/pmol using the ORAC assay. In certain embodiments, the G. buchananii extract is made from the bark of G. buchananii. The bark of G. buchananii can be collected from the stem, root or branches of the plant. In certain embodiments, the bark is collected from the stem of G. buchananii.

The disclosure also provides a composition comprising a fraction of an extract of a component of G. buchananii wherein the extract is enriched in a compound(s) which reduce motility of the gastro-intestinal tract in mammalian subjects. This reduction in motility can be measured by a change in pellet propulsive velocity compared to control. In certain embodiments, the fraction creates a pellet propulsive velocity of less than 75% of control when administered in an effective amount. In other embodiments, the fraction creates a pellet propulsive velocity of between 10 and 80, 25 and 75 or 40-60% when administered in an effective amount. The pellet propulsive velocity can be measured by any method known in the art, either in vitro or in vivo. For example, the pellet propulsive velocity can be measured by pinning segments of guinea pig distal colon (˜12 cm) in a 50 mL organ bath and continuously perfused with oxygenated Krebs solution at 36.5° C. The pellet propulsion velocities and contractile activities can be measured with spatio-temporal maps by using the Gastrointestinal Motility Monitoring system and C-SOF-570 GIMM Software, respectively (GIMM; Med-Associates Inc., Saint Albans, Vt., USA) as described elsewhere.

The disclosure also provides a composition comprising a fraction of an extract of a component of G. buchananii wherein the extract is enriched in a compound(s) which inhibits Ca²⁺ mobilization in smooth muscle cells. This reduction in Ca²⁺ mobilization can be measured by monitoring Ca²⁺ events using a fluorescent Ca²⁺ indicator dye and action potentials and slow waves in smooth muscle cells that are activated through Ca²⁺ mobilization. In certain embodiments, the fraction reduces action potentials to greater than 50% of control when administered to smooth muscle cells in an effective amount. In other embodiments, the fraction reduces action potentials to greater than 10, 20, 30, 40, 50, 60, 70, 80, 90, 95, 96, 97, 98, 99 or 100% of control when administered to smooth muscle cells in an effective amount. In other embodiments, the fraction reduces action potentials between 25 and 75, 30 and 60 or 40 and 50% of control when administered to smooth muscle cells in an effective amount.

The disclosure also provides methods of inhibiting Ca²⁺ mobilization in smooth muscle cells by administering any of the compositions or fractions described above. In certain embodiments, the compositions or fractions are administered in an effective amount as described herein. The reduction in Ca²⁺ mobilization can be measured by monitoring action potentials in smooth muscle cells that are activated through Ca²⁺ mobilization. In certain embodiments, the compositions or fractions reduce action potentials to greater than 50% of control when administered to smooth muscle cells in an effective amount. In other embodiments, the compositions or fractions reduce action potentials to greater than 10, 20, 30, 40, 50, 60, 70, 80, 90, 95, 96, 97, 98, 99 or 100% of control when administered to smooth muscle cells in an effective amount. In other embodiments, the compositions or fractions reduce action potentials between 25 and 75, 30 and 60 or 40 and 50% of control when administered to smooth muscle cells in an effective amount. In certain embodiments, the compositions or fractions are administered to a subject. The subject can be an animal. Animals can include mammals, fish, amphibians, birds and reptiles. Mammals can include rodents, marsupials, ungulates, primates, and carnivores. Examples of mammals also include humans, apes, monkeys, rats, mice, hamsters, gerbils, rabbits, horses, cattle, llamas, camels, dogs, cats, bats, goats and sheep. In certain examples, the subjects are humans. In certain subjects, the subjects are suffering from gastro-intestinal distress as defined herein. These definitions of subjects can be applied to any of the embodiments described, herein.

In certain embodiments, the smooth muscle cells are in vivo. Thus, the compositions and fractions are administered to a subject in vivo. In other embodiments, the smooth muscle cells are in vitro. In these embodiments, the cells are cultured or tissues or organs including smooth muscle cells are cultured. Examples of methods for culture of these cells are provided herein.

The disclosure also provides methods of reducing the redox state of a biological system by administering any of the compositions or fractions or combinations thereof described above. The biological system can be selected from a cell, a tissue, an organ and an organism. The reduction of the redox state (making the redox state less oxidized) of a biological system is performed by administering one or more compositions or fractions that have antioxidative properties in an effective amount. The antioxidative properties of a given composition or fraction can be measured according to any method known in the art. For example, the antioxidative properties of a fraction or composition could be measured using the H₂O₂ or ORAC methods described herein.

The disclosure also provides methods of treating gastro-intestinal distress in a subject in need thereof comprising administering any of the compositions or fractions or combinations thereof described above. In certain embodiments, gastro-intestinal distress includes diarrhea. In some embodiments, diarrhea includes secretory diarrhea, osmotic diarrhea, exudative diarrhea, motility-related diarrhea, or inflammatory diarrhea. Diarrhea can be caused by infection, auto-immune problems, inflammatory bowel disease, maldigestion or diet. In other embodiments, gastro-intestinal distress also includes dysentery. Gastro-intestinal distress can also include diarrhea, colitis, ulcerative colitis, inflammatory bowel disorder, irritable bowel syndrome, ischemic bowel disease, Crohn's disease, colitis due to acute and chronic intestinal ischemia, hormone-secreting tumors, ulcerative colitis, celiac disease, Whipple disease, Graft-versus-Host disease after stem cell transplantation, food poisoning, dysentery, viral gastroenteritis, food allergy, food intolerance, bile acid malabsorption and infection. Infection can be caused by certain strains of E. coli, norovirus, rotavirus, adenovirus (types 40 and 41), astroviruses, Campylobacter species, particularly jejuni, Salmonella, Shigella, Giardia, Cholera, Clostridium difficile, Staphylococcus aureus, Entamoeba histolytica, Cryptosporidium.

The disclosure also provides methods of treating pain in a subject in need thereof comprising administering any of the compositions or fractions or combinations thereof described above. In certain embodiments, the fractions are M4 and/or M5 and the compounds are (2R,3R,2″R,3″R) GB-2 and/or 2R,3S,2″S) buchananiflavanone. The disclosure also provides all stereoisomers and enantiomers of (2R,3R,2″R,3″R) GB-2 and/or 2R,3S,2″S) buchananiflavanone.

The disclosure also provides methods of treating gastro-intestinal distress in a subject in need thereof comprising administering any of the compositions or fractions or combinations thereof described above along with one or more known treatments for diarrhea. These treatments can include oral rehydration salts, antibiotics, bismuth compounds, anti-motility agents and bile acid sequestrants. The disclosure also provides dosage forms and kits that combine any of the compositions or fractions or combinations thereof described above along with one or more known treatments for diarrhea.

The disclosure also provides methods of treating pain in a subject in need thereof comprising administering any of the compositions or fractions or combinations thereof described above along with one or more known treatments for pain. These treatments can include opiate or non-opiate pain relievers. The disclosure also provides dosage forms and kits that combine any of the compositions or fractions or combinations thereof described above along with one or more known treatments for diarrhea.

The disclosure also provides methods of isolating any of the compounds of or fractions described above. In one embodiment, the method includes producing an extract from G. buchananii bark comprising water and ethanol; fractionating the extract using PTLC or MPLC; and isolating fractions with antioxidant activity, thereby isolating any of the compounds or fractions described above. In certain embodiments of this method, the water is present in the extract at between 5 and 50%. In other embodiments of this method, the fractionation splits the extract into between 3 and 10 or 5 and 8 fractions. In other embodiments of this method, the extract was filtered, extracted with hexane, evaporated and resuspended in a water/methanol mixture. In other embodiments of this method, the fractionation was performed by MPLC, PTLC, HPLC or solvent fractionation. In certain embodiments, the solvent fractionation is performed using ethyl acetate. Other methods of isolating any compounds or fractions described herein are also provided below in the Detailed Description and in the Examples.

The disclosure also provides methods of treating constipation in a subject in need thereof comprising administering semi-refined fractions (PTLC2 and M6) from G. buchananii stem bark extract.

In certain embodiments, the isolated compound is present in a fraction of a component of G. buchananii wherein the fraction is enriched in the compound having prokinetic effects. In certain embodiments, the subject is a mammalian subject. The mammalian subject can be a human. In other embodiments, the component of G. buchananii is the bark of G. buchananii. The bark can be isolated from the stem, roots or branches of G. buchananii. The fraction can be isolated using preparative thin layer chromatography (PTLC), medium pressure liquid chromatography (MPLC) and high pressure liquid chromatography

The disclosure also provides methods of treating constipation in a subject in need thereof comprising administering the PTLC2 fraction from G. buchananii stem bark extract. In certain embodiments, the subject is a mammalian subject. The mammalian subject can be a human.

The disclosure also provides methods of treating diarrhea by administering less than 10 g of G. buchananii bark powder per 100 mL Krebs to a mammalian subject. In certain embodiments, the administration is between 0.1 g G. buchananii bark powder per 100 mL Krebs and 10 g of G. buchananii bark powder per 100 mL Krebs. In other embodiments, the administration is between 1 and 8, 2 and 7, 3 and 6 or 4 and 5 g of G. buchananii bark powder per 100 mL Krebs. The mammalian subject can be a human. In certain embodiments, the mammalian subject can include rodents, marsupials, ungulates, primates, and carnivores. Examples of mammals also include humans, apes, monkeys, rats, mice, hamsters, gerbils, rabbits, horses, cattle, llamas, camels, dogs, cats, bats, goats and sheep. In certain examples, the subjects are humans. In certain subjects, the subjects are suffering from gastro-intestinal distress as defined herein. These definitions of subjects can be applied to any of the embodiments described, herein. The bark of G. buchananii can be collected from the stem, root or branches of the plant. In certain embodiments, the bark is collected from the stem of G. buchananii.

### BRIEF DESCRIPTION OF THE DRAWINGS

FIG. 1. Intraluminal and bath applications of G. buchananii bark extract inhibit pellet propulsion in isolated segments of guinea pig distal colon. (A) Bar graph showing the effects of intraluminal VS bath delivery of the extract (1 g bark powder/100 mL Krebs solution; 5-min application) on propulsive motility. No difference was detected between intraluminal and bath applications of the vehicle with regard to pellet velocity (bracket with ns). Intraluminal and bath extract applications reduced pellet velocity (bracket with asterisk). 1 g powder in 100 mL Krebs had larger inhibitory effects when applied in the bath vs an intraluminal delivery (bracket with triangle). (B) The concentration-dependent effects of the extract and the effects of washout on pellet propulsion. Lower concentrations (0.01-0.1 g bark powder/100 mL Krebs) did not alter motility. Higher concentrations (1-10 g/100 mL Krebs) inhibited propulsion in a concentration-dependent fashion. At concentrations of 1 g bark powder/100 mL Krebs and below, pellet propulsion was rapidly restored to normal values and an increased rate of propulsive motility (30-100% above baseline) was observed following washout for 10-20 min Normal pellet propulsion was not restored by 20 mins of washout after treatment with G. buchananii bark powder at a concentration of 10 g/100 mL Krebs.

FIG. 2. Demonstration that evoked fast excitatory postsynaptic potentials (fEPSPs) activity was inhibited by application of G. buchananii extract (0.25-0.5 g bark powder/100 mL Krebs), and these actions were not mediated by opioid or alpha-2 adrenoceptor receptors. (A) Representative traces of fEPSPs, demonstrating that G. buchananii extract inhibited fEPSPs in S-neurons in the myenteric ganglia of guinea pig distal colon. These actions were not affected by the opioid receptor antagonist, naloxone (10 μmol L⁻¹), or by the alpha-2 adrenoceptor antagonist, yohimbine (1 μmol L⁻¹). (B) Summary data showing that G. buchananii bark extract (0.25 g bark powder/100 mL Krebs) reduced fEPSPs amplitudes in the myenteric neurons after 5 min (bracket with triangle). The inhibitory actions of G. buchananii extract on fEPSPs persisted in the presence of the opioid receptor antagonist, naloxone (10 μmol L⁻¹) and the alpha-2 adrenoceptor antagonist, yohimbine (1 μmol L⁻¹) (bracket with asterisk). The fEPSPs amplitudes were restored to normal values by washout for 5-10 mins, and were not affected by naloxone (ns bracket).

FIG. 3. Inhibitory actions of G. buchananii extract (2 g bark powder/100 mL Krebs) on pellet motility do not involve opioid or α-2 adrenergic receptor activation. (A-B) Summary data showing that neither naloxone (10 μmol L⁻¹; A, bracket with triangle) nor yohimbine (1 μmol L⁻¹; B, bracket with triangle) altered the ability of G. buchananii extract to reduce propulsive motility. The rebound prokinetic effect of G. buchananii extract detected after washout also persisted in the presence of naloxone and yohimbine (A-B, brackets with asterisks).

FIG. 4. Garcinia buchananii extract contains bioactive components that act as GABA receptor ligands. (A-B) Summary data showing that GABA (50 μmol L⁻¹, A, triangle), the GABA_{B} receptor antagonist, phaclofen (PCF, 40 μmol L⁻¹; B, star), and the GABA_{A} receptor antagonist picrotoxin (PTX, 30 μmol L⁻¹; B, square), did not alter the effects of G. buchananii extract (2 g bark powder/100 mL Krebs, delivered by bath application) on propulsive motility after 5-10 mins Recovery of propulsive motility by post-treatment washout was similar to vehicle results for GABA treatment (increasing propulsive motility by up to 70% above baseline at 15-20 mins washout intervals; (A, bracket with asterisk; P<0.05). A slower recovery was observed during washout after treatment using the extract in combination with picrotoxin (PTX, 30 μmol L⁻¹) or phaclofen (PCF, 40 μmol L⁻¹). In these cases, motility was still significantly lower than that of vehicle after 20 mins of washout (bracket with asterisk in B; P<0.05).

FIG. 5. Preparative thin layer chromatography (PTLC) was used to separate and isolate fractions of the bark extract. Five fractions were isolated using PTLC. These fractions were labeled as PTLC 1-5. (A) Preparative thin-layer chromatography (PTLC) separation of aqueous G. buchananii extract and fraction identification based on color indexes. Extract sample indicates the region where 100 μL of extract was applied using a CAMAG Linomat 5 sample applicator. PTLC1-5 designate the five fractions isolated from aqueous G. buchananii extract. PTLC silica shows a region used to collect silica for control experiments. (B) Separation of aqueous G. buchananii extract (extract) and further separation of PTLC1-5 fractions using high-performance thin-layer chromatography. Each PTLC fraction contained a minimum of three distinct bands.

FIG. 6: A series of charts that show results of UPLC-TOF-MS analysis of PTLC fractions and Garcinia buchananii extract from top to bottom.

FIG. 7: Characterization of the effect of aqueous G. buchananii extract fractions on guinea pig colon motility using pellet propulsion assays in isolated distal colons. Summary data (20 min application to the serosal surface) showing that PTLC fractions contain components with anti-motility and pro-motility effects. PTLC1, PTLC5, and a combination of PTLC1+PTLC5 (*; P<0.05; see Table 1) significantly inhibited pellet propulsion. The effects of PTLC1 and PTLC5 were not additive. PTLC2 increased pellet propulsion (Δ; P<0.05). Compared with control (PTLC silica), PTLC3 and PTLC4 did not alter propulsive velocity (P>0.05).

FIG. 8: The anti-motility effects of aqueous G. buchananii bark extract elicited via mucosal surface application depend on 5-HT₄ and 5-HT₃ receptors. (A) Summary data (20 min after application) showing that compared with vehicle, G. buchananii extract (2 g bark powder/100 mL Krebs) significantly inhibited pellet velocity (*P<0.001). Serotonin hydrochloride (5-HT; 0.5 μmol L⁻¹), CJ-033466 (300 nmol L⁻¹), cisapride (CISA; 100 nmol L⁻¹), and RS-56812 (50 nmol L⁻¹) all individually increased pellet velocity beyond that of vehicle (A; each P<0.05) and 2 g extract alone ( [symbol] ; each P<0.001). Compared with G. buchananii extract alone (2 g ext.), the endogenous 5-HT agonist 5-HT, 5-HT₄ receptor agonists cisapride (CISA) and CJ-033466, and 5-HT₃ receptor agonists RS-56812 significantly reduced the potency of the extract to inhibit pellet propulsion (♦; each P<0.05). (B) Summary data (20 min after application) showing that compared with vehicle, 2 g bark powder/100 mL Krebs) significantly reduced pellet velocity (*; P<0.001). Furthermore, intraluminal application of the 5-HT₃ receptor antagonist granisetron (GRAN; 0.5 μmol L⁻¹) and GRAN+2 g extract significantly reduced pellet velocity (Δ; each P<0.05). In contrast, the 5-HT₄ receptor antagonist (GR-113808) and 2 g extract plus GR-113808 did not reduce pellet velocity. Compared with G. buchananii extract (2 g ext.) alone, a combination of 2 g extract with granisetron and GR-113808 significantly reduced the effectiveness of G. buchananii extract to inhibit motility (*; P<0.05).

FIG. 9: 5-HT₃ and 5-HT₄ receptor agonists reverse the anti-motility effects elicited by G. buchananii bark extract when applied to the serosal surface. (A) Summary data (20 min after application) showing that compared with vehicle, 1 g G. buchananii (1 g ext.) significantly inhibited pellet motility by 90% (*P<0.001) and the 5-HT₄ receptor agonist, cisapride (CISA: 100 nmol L⁻¹) inhibited pellet propulsion by 15% (Δ; P<0.05). Unlike intraluminal applications (see FIG. 8A), the 5-HT₃ receptor agonist RS-56812 (50 nmol L⁻¹) and the 5-HT₄ receptor agonist CJ-033466 (CJ, 300 nmol L⁻¹) did not alter pellet velocity (P>0.05). In addition, the extract (1 g ext.) did not inhibit pellet propulsion in the presence of 5-HT₃ receptor agonist, RS-56812 and 5-HT₄ receptor agonists cisapride and CJ-033466 (♦; P<0.001). Interestingly, the combination of G. buchananii extract with CJ-033466 increased pellet velocity when compared with vehicle and cisapride ( [symbol] ; P<0.05 and P<0.01, respectively). (B) Summary data (20 min after application) showing that compared with vehicle, G. buchananii extract (1 g ext.) inhibited pellet propulsion (*P<0.001). A mixture of 5-HT₃ agonist RS-56812 and 5-HT4 agonist cisapride (CISA) increased pellet velocity (Δ; P<0.05). Compared with extract alone, when colons were treated with 1 g G. buchananii extract combined with cisapride and RS-56812, the extract failed to reduce pellet motility ( [symbol] , P<0.001). Instead, the combination increased pellet velocity beyond that of vehicle alone (♦; P<0.05). (C) In serosal applications (20 min after application), 5-HT₃ and 5-HT₄ receptor antagonists did not alter the anti-motility effect of G. buchananii. G. buchananii extract (1 g ext.) inhibited pellet propulsion (*P<0.001). A combination of G. buchananii extract with the 5-HT₃ antagonists ondansetron (ONDAN, 0.5 μmol L⁻¹) and granisetron (GRAN, 1 μmol L⁻¹) and 5-HT₄ receptor antagonists GR-113808 (GR-113808, 5 μmol L⁻¹) inhibited pellet propulsion with similar magnitude to that of the extract alone (Δ; P<0.05). GR-113808 appeared to augment the extract's actions, whereas 5-HT₃ receptor antagonists tended to inhibit the effects of the extract by 5-10%. (D) A combination of the 5-HT₃ receptor antagonist, ondansetron (ONDAN; 0.5 μmol L⁻¹) with 5-HT₄ receptor antagonist GR-113808 (5 μmol L⁻¹) did not affect pellet propulsion for 20 min (P>0.05). However, mixing ondansetron and GR-113808 with G. buchananii extract (1 g ext.) reduced the anti-motility potency of G. buchananii extract by 40% (♦; P<0.05).

FIG. 10: 5-HT₃ and 5-HT₄ receptor agonists and antagonists affect the anti-motility effectiveness of PTLC1 and PTLC5 fractions elicited from serosal side of guinea-pig distal colon. (A) Summary data showing that compared with PTLC silica, PTLC1 (15 mg per 100 mL Krebs) inhibited pellet propulsion by 25% after 20 min (*P<0.05). As with G. buchananii extract, in the presence of the 5-HT₄ agonists cisapride (CISA; 100 nmol L⁻¹) and CJ-033466 (300 nmol L⁻¹), PTLC1 did not alter pellet propulsion (P>0.05). In contrast, PTLC1 inhibited pellet propulsion by 35% in the presence of the 5-HT₃ receptor agonist RS-56812 (50 nmol L⁻¹) (♦; P<0.01). (B) Summary data showing that compared with PTLC silica PTLC5 (3.8 mg per 100 mL Krebs) inhibited pellet propulsion by 20% after 20 min (*; P<0.05). Unlike PTLC1, PTCL5 inhibited pellet propulsion in the presence of the 5-HT₄ receptor agonist cisapride (CISA; 100 nmol L⁻¹) (Δ; P<0.05). As with PTLC1 and G. buchananii extract, in the presence of the more specific 5-HT₄ receptor agonist CJ-033466 (300 nmol L⁻¹), PTLC5 did not alter pellet propulsion (P>0.05). The pellet propulsion velocity obtained in the presence of PTLC5 combined with CJ-033466 was higher than that of PTLC5 alone (♦; P<0.05). Furthermore, PTLC5 did not reduce pellet propulsion in the presence of the 5-HT₃ receptor agonist RS-56812 (50 nmol L⁻¹; P>0.05), which contrasts with PTLC1. (C) Summary data (20 min application) showing that compared with PTLC silica, PTLC1 alone (15 mg per 100 mL Krebs) inhibited pellet propulsion by 25% (*P<0.05) and by 58% in the presence of 5-HT₄ antagonist GR-113808 (Δ; P<0.001). GR-113808 augmented the effects the fractions by 30%. In contrast, PTLC1 did not inhibit pellet propulsion in the presence of 5-HT₃ receptor antagonists, granisetron and ondansetron (each 1 μmol L⁻¹; P>0.05). Instead, combining PTLC1 with ondansetron increased propulsive motility beyond that of PTLC silica, PTLC1 alone ( [symbol] ; P<0.05). Combining PTLC1 with granisetron significantly reduced the anti-motility potency of PTLC1 when compared with a mixture of PTLC1 with the 5-HT₄ antagonist GR-113808 (♦; P<0.05). (D) Summary data (20 min application) showing that compared with PTLC silica, PTLC5 alone inhibited pellet propulsion by 20% (*; P<0.05) and by 50% in the presence of 5-HT₄ antagonist GR-113808 (Δ; P<0.001). As with PTLC1, GR-113808 augmented the effects the PTLC5 by 30%. Similar to PTLC1, PTLC5 did not inhibit pellet propulsion in the presence of 5-HT₃ receptor antagonists, ondansetron and granisetron (each 1 μmol L⁻¹); P>0.05). Pellet propulsion velocity obtained when PTLC5 was applied in combination with granisetron was significantly greater than that of PTLC5 alone and PTLC5 mixed with GR-113808 (♦; P<0.05 and P<0.001, respectively). PTLC5 mixed with GR-113808 caused a significantly reduced propulsion velocity when compared with PTLC5 plus odansetron ( [symbol] ; P<0.001).

FIG. 11 shows bar graphs demonstrating decreases in mechanosensitivity due to administration of 0.5 g/100 ml Krebs of a Garcinia buchananii extract in in vitro mouse colon preparations.

FIG. 12: Aqueous G. buchananii bark extract prevents fluid loss via stools. The 35% HLD caused a dramatic increase in fecal fluid content during the inducement period (see days 2-6). Compared with non-treated rats (lactose), all doses of G. buchananii bark extract (0.1 and 1.0 g bark powder/L) significantly reduced fecal fluid content after one day (day 7; P<0.05 and P<0.01, respectively). Similarly, loperamide significantly reduced fecal fluid content after 1 day (P<0.01). An increased reduction of fecal fluid content was seen during subsequent treatment days (2-4 days) with the extract at doses of 0.1 and 1.0 g/L (see days 8-10; P<0.01 and P<0.001, respectively). Treatments using 1.0 g extract and loperamide reduced fecal fluid content to normal levels observed in rats fed a standard chow diet (SD; n=3). After 3-4 days (see days 8-10) of treatment, fluid content in feces from HLD rats treated with 0.1 g extract was greater than that of SD rats (P<0.05). However, it was not different from HLD rats treated with 1.0 g bark powder/L extract or loperamide (8.4 mg/L; n=4; P>0.05). All statistical comparisons were performed using 1-way ANOVA.

FIG. 13: G. buchananii extract treatment and loperamide did not increase the number of fecal pellets (defecation rate) in rats with diarrhea due to HLD after 4 days of treatment. A high lactose diet (HLD) caused rats to produce watery or loose, mucoid, sticky, and yellowish stools significantly reducing the number of pellets beyond that of rats fed a control standard chow diet (SD) (+; P<0.001; 1-way ANOVA). Although not significantly different, the numbers of pellets produced by rats treated with aqueous G. buchananii bark extract (0.1-5.0 g/L) were 2-3 times greater than those produced by untreated rats on the HLD or HLD rats treated with loperamide.

FIG. 14 shows the effects of G. buchananii bark extract on weight loss, food consumption and fluid consumption in HLD rats with diarrhea.

FIG. 14A: The HLD caused significant loss of weight compared with the standard chow diet (SD; [symbol] ; P<0.001). The weight gain of SD rats was significantly greater than those of all the anti-diarrhea treatments (Δ; P<0.001). 0.1 g G. buchananii bark extract, not only reversed the HLD induced weight loss but actually caused a weight gain (*; P<0.05). G. buchananii bark extract at 0.5 g/L and 1.0 g/L showed trends of a reduced weight loss compared with HLD although this did not reach significance (P>0.05), whilst 5 g/L was similar to HLD alone (P>0.05).

FIG. 14B: The aqueous G. buchananii bark extract did not improve overall food consumption in rats with HLD-induced diarrhea. A HLD significantly reduced food consumption ( [symbol] ; P<0.001). Likewise, food consumption was reduced in diarrheic rats treated with G. buchananii bark extract (all doses) and loperamide (A; P<0.001).

FIG. 14C: A HLD did not cause a significant decline in fluid consumption compared with SD alone. Similarly, lower doses (0.1 g/L and 1.0 g/L) of G. buchananii bark extract did not significantly alter fluid consumption. However, 5.0 g/L G. buchananii bark extract and loperamide significantly reduced water intake (♦; P<0.01).

FIG. 15 shows the effects of treating HLD induced diarrheic rats with anti-motility fractions, PTLC1 (15 mg/100 mL) and PTLC5 (3.8 mg/100 mL) animal weights (A), food consumption (B) and water intake (C).

FIG. 15A: PTLC1 and PTLC5 both reversed the HLD induced weight loss and promoted weight gain. The HLD caused significant weight loss compared with SD alone ( [symbol] ; P<0.001). PTLC1 and PTLC5 both reversed the HLD induced weight loss and promoted weight gain (*; P<0.001; Δ; P<0.001).

FIG. 15B: PTLC1 and PTLC5 both increased food consumption in HLD induced diarrheic rats. Compared with SD rats, a HLD significantly reduced rats' food consumption ( [symbol] ; P<0.001). PTLC1 and PTLC5 both reversed the effect of a HLD and increased food consumption in diarrheic rats (*; P<0.001 and Δ; P<0.01; respectively). PTLC1 increased food intake compared with SD (♦; P<0.01).

FIG. 15C: PTLC5 increased fluid consumption in HLD induced diarrheic rats. The HLD did not cause a significant decline in water consumption compared with SD rats (P>0.05). PTLC5 significantly increased fluid consumption when compared with PTLC1 (*; P<0.01) as well as SD and untreated (HLD) rats (Δ; P<0.05 and P<0.001; respectively)

FIG. 16: Garcinia buchananii bark extract reduces intestinal bloating. Pictures showing that compared to standard chow diet (SO), a high lactose diet (HLD) caused bloating and distension of cecum and colon in rats. Rats on a SD had smaller cecum sizes and their colons were filled with fecal pellets. Rats on a HLD had bloated and distended distal ileum, cecum and colon. All doses of G. buchananii extract and loperamide reduced cecum distension (size).

FIG. 17 shows the structure of antioxidant compounds isolated from fractions of Garcinia buchananii stem bark extract.

FIG. 18 is a line graph depicting high resolution LC-TOF-MS analysis of compound 1 (also known as antioxidative compound 1, herein) as well as its ¹H NMR spectrum.

FIG. 19 is a chart showing a 2D NMR spectrum representing compound 1.

FIG. 20 shows two line graphs showing circular dichromism spectroscopic measurements of the commercially available reference isomer (+)-(2R,3R)-taxifolin (6) as well as the isolated C-glycosides 1 and 2 (compounds 1 and 2).

FIG. 21 is a chart showing a 2D NMR spectrum representing compound 3.

FIG. 22 shows two line graphs showing circular dichromism spectroscopic measurements of compounds 3, 4 and 5.

FIG. 23A shows the results of medium pressure liquid chromatographic separation of an ethanol/water G. buchananii extract separating the extract into eight fractions M1-M8.

FIG. 23B is a schematic showing compounds 1-5 isolated from their respective fractions M1-M5. Compounds 1-5 were the main components of their respective fractions.

FIG. 24A is a graph showing percent pellet propulsive velocity in Krebs and DMSO controls compared to the M1, M2 and M3 fractions. M1, M2 and M3 did not affect pellet propulsion velocity.

FIG. 24B is a graph showing percent pellet propulsive velocity in Krebs and DMSO controls compared to the M6 and M8 fractions. M6 and to some extent M8 increased pellet propulsion velocity in isolated guinea pig colons, which is similar to the findings obtained using PTLC2.

FIG. 25A is a graph showing percent pellet propulsive velocity in Krebs control compared to the M4 and M5 fractions. M4 inhibited pellet propulsion.

FIG. 25B is a spatial temporal map of a pellet propulsion pattern in a colon segment (distance versus time) from oral end (top left) to aboral end (bottom right) with control administered. Under control conditions the propulsion is linear.

FIG. 25C is a spatial temporal map of a pellet propulsion pattern in a colon segment (distance versus time) from oral end (top left) to aboral end (bottom right) with M4 administered. M4 transiently reduced pellet propulsion ten minutes after application. M4 reduced colon diameter and aboral relaxation.

FIG. 26A is a graph showing percent pellet propulsive velocity in DMSO control compared to the quercetin and the M5 fractions. M5 inhibited pellet propulsion.

FIG. 26B is a spatial temporal map of a pellet propulsion pattern in a colon segment (distance versus time) from oral end (top left) to aboral end (bottom right) with control administered. Under control conditions the propulsion is linear.

FIG. 26C is a spatial temporal map of a pellet propulsion pattern in a colon segment (distance versus time) from oral end (top left) to aboral end (bottom right) with M7 administered. M7 transiently reduced pellet propulsion ten minutes after application.

FIG. 27 is a chart showing the results of high performance liquid chromatographic separation of ethanol/water MPLC fraction M7 into subfractions M7-1 to M7-4.

FIG. 28 is a graph showing percent pellet propulsive velocity for the M7-3 and M7-4 fractions. M7-4 inhibited pellet propulsion.

FIG. 29A is a chart showing results from a time of flight mass spectroscopy chromatogram showing the various peaks and masses for compounds in the M7 fraction.

FIG. 29B is a chart showing results from a time of flight mass spectroscopy chromatogram showing the various peaks and masses for compounds in the M7-4 fraction. The masses of 541 and 557 suggest that the candidate compounds with antimotility effects are biflavanones similar in structure to manniflavanone, GB-2 and buchananiflavanone but have less oxygen. The compound with a mass of 929 is probably also a flavanone, a special flavanone derivative and is unknown. 243 is most likely a 1,5-dihydroxy-2,3-dimethoxyxanthone.

FIG. 30 is a bar graph showing the effect of G. buchananii stem bark extract on mechanosensory neurons in guinea pig ileum. G. buchananii inhibits neurotransmission in mechanosensory neurons.

FIG. 31 is a bar graph showing the effect of G. buchananii stem bark extract MPLC fractions M4 and M5 on mechanosensory neurons in guinea pig ileum. M4 and M5 inhibit neurotransmission in mechanosensory neurons.

FIG. 32A is a chart showing representative traces of evoked inhibitory junction potentials (IJPs) in colon smooth muscle. Junction potentials were evoked in impaled circular muscle cell by transmural electrical field stimulation in whole mounts pinned 1.5-2.5 cm between stimulating electrodes. This chart shows baseline, treatment with 6 mg/mL of M4 in Krebs for 15 minutes and after 15 minutes of washout.

FIG. 32B is a chart showing representative traces of evoked IJPs in colon smooth muscle. Junction potentials were evoked in impaled circular muscle cell by transmural electrical field stimulation in whole mounts pinned 1.5-2.5 cm between stimulating electrodes. This chart shows baseline, treatment with M5 in Krebs for 10 minutes and after 20 minutes of washout.

FIG. 32C is a chart showing representative traces of evoked IJPs in colon smooth muscle. Junction potentials were evoked in impaled circular muscle cell by transmural electrical field stimulation in whole mounts pinned 1.5-2.5 cm between stimulating electrodes. This chart shows baseline, treatment and with 6.6 mg/mL of M7 in Krebs for 10 minutes.

FIG. 33A is a chart showing traces of antidromic action potentials in an AH neuron were elicited by direct stimuli (a single pulse of 0.5 ms) applied to a fiber tract in the interganglionic nerve strand while holding a cell at −90 mV. Compared with the vehicle and washout, more antidromic action potentials were elicited in the presence of the extract (n=2 cells).

FIG. 33B shows charts demonstrating the effects of direct current injection (0.2 nA; 500 ms) into AH neurons. Direct current injection during baseline recording and in the presence of G. buchananii had similar effects.

FIG. 34 is a bar graph showing the relative antioxidative activity (ORAC assay) of fractions M1-M7 of G. buchananii stem bark extract.

FIG. 35 is a series of charts showing the quantitation of the main antioxidative compounds in G. buchananii via UPLC-TOF-MS analysis. From top to bottom: extracted ion chromatograms of accurate masses of (2R,3R)-Taxifolin-6-C-β-D-glucopyranoside, (2R,3S,2″R,3″R)-GB-2 and (2R,3S,2″S)-Buchananiflavanone, as well as (2R,3S,2″R,3″R)-manniflavanone. A mass window of 10 mDa for all extracted ions was used. Down BPI: (base peak ion mass chromatogram) of the whole extract.

FIG. 36A is a bar graph showing the concentration of (2R,3R)-Taxifolin-6-C-β-D-glucopyranoside, (2R,3S,2″R,3″R)-manniflavanone, (2R,3S,2″R,3″R)-GB-2 and (2R,3S,2″S)-Buchananiflavanone in G. buchananii stem bark extract.

FIG. 36B lists the calculated amount of antioxidative activity (ORAC assay) for each compound in 100 mg of extract and the concentrations of antioxidative compounds in 100 mg G. buchananii stem bark extract.

FIG. 37 is a bar graph showing the relative antioxidative activity (ORAC assay) of fractions M1-M8 of G. buchananii stem bark extract, combinations of these fractions and of (2R,3R)-Taxifolin-6-C-β-D-glucopyranoside, (2R,3S,2″R,3″R)-manniflavanone, (2R,3S,2″R,3″R)-GB-2 and (2R,3S,2″S)-Buchananiflavanone.

FIG. 38 is a bar graph showing the relative antioxidative activity (H₂O₂ assay) of fractions M1-M8 of G. buchananii stem bark extract and of (2R,3R)-Taxifolin-6-C-β-D-glucopyranoside, (2R,3S,2″R,3″R)-manniflavanone, (2R,3S,2″R,3″R)-GB-2 and (2R,3S,2″S)-Buchananiflavanone.

FIG. 39 is a bar graph showing the relative antioxidative activity (H₂O₂ assay) of fractions M1-M8 of G. buchananii stem bark extract, combination of fractions M1-M8 and combination of (2R,3R)-Taxifolin-6-C-β-D-glucopyranoside, (2R,3S,2″R,3″R)-manniflavanone, (2R,3S,2″R,3″R)-GB-2 and (2R,3S,2″S)-Buchananiflavanone thereof.

FIG. 40A shows a micrograph of guinea pig gall bladder smooth muscle fascicles and traces of Ca²⁺ flashes generated from selected smooth muscle cells wherein the cells are administered a control.

FIG. 40B shows a micrograph of guinea pig gall bladder smooth muscle fascicles and traces of Ca²⁺ flashes generated from selected smooth muscle cells wherein the cells are administered 0.5 g of G. buchananii stem bark powder/100 ml of physiological saline solution.

FIG. 40C is a bar graph showing the reduction of Ca²⁺ flashes in smooth muscle cells in intact full thickness (*P<0.01) and muscularis (*P<0.001) gallbladder preparations from guinea pigs when treated with G. buchananii stem bark extract (0.5 g/100 ml PSS).

FIG. 40D is a bar graph showing that G. buchananii extract inhibits Ca²⁺ flashes in smooth muscle cells in muscularis externa preparations of the guinea pig distal colon.

FIG. 41A is shows traces of action potentials (AP) from guinea pig gallbladder smooth muscle cells (GBSM; standard intracellular microelectrode recording in intact tissues) showing that compared with Krebs solution (vehicle), G. buchananii stem bark extract (0.5 g/100 ml vehicle) inhibits spikes of AP (rapid membrane depolarizations due to Ca²⁺ influx via L-type calcium channels) and sub-threshold membrane depolarizations (arrows).

FIG. 41B shows the effect of G. buchananii stem bark extract on slow wave action potentials (SW) and membrane potentials in guinea pig colon smooth muscle cells. Representative traces of SW showing that compared with Krebs vehicle, G. buchananii extract from 0.5 g stem bark powder/100 ml Krebs inhibits SW in circular smooth muscle cells in intact preparations of muscularis externa. Washout rapidly restores SW. The rhythmicity and synchronicity of SW returns to normal after a washout.

FIG. 42A shows traces of action potentials from gallbladder smooth muscle cells during superfusion with Krebs vehicle at 36.5 degrees Celcius.

FIG. 42B shows traces of action potentials from gallbladder smooth muscle cells demonstrating that manniflavanone inhibits the discharge of action potentials. Manniflavanone inhibits the discharge of spikes before eliminating sub-threshold membrane depolarizations (arrow).

FIG. 42C shows traces of sub-threshold membrane depolarizations (arrow, 10 min) and action potentials (15 min) from gallbladder smooth muscle cells showing restoration of electrical activity after washout of manniflavanone.

FIG. 43A shows traces of slow wave action potentials (SW) from smooth muscle cells of the inner circular layer of guinea pig colon muscularis externa in the presence of Krebs vehicle.

FIG. 43B shows traces of slow wave action potentials from smooth muscle cells of the inner circular layer of guinea pig colon muscularis externa manniflavanone. Manniflavanone inhibited the discharge of sub-threshold membrane depolarizations and superimposed spikes.

FIG. 43C shows traces of slow wave action potentials from smooth muscle cells of the inner circular layer of guinea pig colon muscularis externa after washout of manniflavanone. Washout restores SW activity.

FIG. 44A is a bar graph showing colonic afferent recordings with G. buchananii stem bark extract fraction M4.

FIG. 44B is a bar graph showing colonic afferent recordings with G. buchananii stem bark extract fraction M5.

FIG. 45 is A chromatogram of high pressure liquid chromatographic separation of G. buchananii matched with PTLC 1 and PTLC 5 fractions to indicate chromatograms containing these fractions.

FIG. 46 is a trace showing results from an Ussing voltage claim method on human mucosal/submucosal preparations.

FIG. 47 is a bar graph showing the pro-secretory effect of G. buchananii [0.3 mg/ml] in mucosal/submucosal preparations of guinea pig distal colon.

FIG. 48 is a trace showing the anti-motility action of MPLC fraction 1 (M1; (2R,3R)-taxifolin-6-C-β-D-glucopyranoside) from G. buchananii in humans.

### DETAILED DESCRIPTION OF THE INVENTION

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

According to some embodiments, there is provided an extract prepared from G. buchananii, which is enriched for an activity that inhibits motility of the colon. In some embodiments, the extract prepared from G. buchananii is enriched for an activity that inhibits motility from both the mucosal and serosal surfaces of the colon. In some embodiments, the extract prepared from G. buchananii is enriched for an activity which reduces propulsive motility through inhibition of S-neuron synaptic activity. In some embodiments, the extract prepared from G. buchananii is enriched for an activity that has a serotonergic effect. In some embodiments, the extract prepared from G. buchananii is enriched for a pro-motility effect. In some embodiments, the extract prepared from G. buchananii is enriched for bioactive compounds that interact with 5-HT₃ and 5-HT₄ receptors to inhibit peristaltic activity. In some embodiments, the extract prepared from G. buchananii is enriched for bioactive compounds that reduce colon motility. In some embodiments, the extract prepared from G. buchananii is enriched for polyphenol and/or flavonoid compounds. In some embodiments, the extract prepared from G. buchananii is enriched for flavonoids. In some embodiments, the extract prepared from G. buchananii is enriched for flavanoids. In some embodiments, the extract prepared from G. buchananii is enriched for xanthones. In some embodiments, the extract prepared from G. buchananii is enriched for flavonoids, tannins and steroids. In some embodiments, the extract prepared from G. buchananii is enriched for flavonoids, tannins, phenols and steroids. In some embodiments, the extract prepared from G. buchananii is enriched for biflavanones, flavonoids, glycosides, alkaloids, tannins, phenols and steroids. In some embodiments, the extract prepared from G. buchananii is enriched for flavonoids, xanthones, glycosides, or alkaloids. In some embodiments, the extract prepared from G. buchananii is enriched for flavonoids, steroids, alkaloids, tannins and phenols. In some embodiments, the extract prepared from G. buchananii is enriched for flavonoids, phenolics, creosol, guaiacol and 4-ethylguaiacol. In some embodiments, the extract prepared from G. buchananii is enriched for flavonoids and/or phenols. In some embodiments, the extract prepared from G. buchananii is enriched for flavonoids and/or phenols further comprising steroids, alkaloids and tannins.

In some embodiments, the extract prepared from G. buchananii is enriched by at least about 500- to 1500-fold for the anti-propulsive motility activity, as compared with the initial aqueous extracts which are 100% derived from G. buchananii. The extract may be enriched to a similar degree in the concentration of flavanones, flavonoid and other polyphenol compounds detected by spectroscopic methods. In some embodiments, the extract prepared from G. buchananii is enriched by at least about 20- to 150-fold for the anti-propulsive motility activity, as compared with the initial aqueous extracts which are 100% derived from G. buchananii. The extract may be enriched to a similar degree in the concentration of flavonoid and other polyphenol compounds detected by spectroscopic methods. In some embodiments, the extract prepared from G. buchananii is enriched by at least about 50- to 500-fold for the anti-propulsive motility activity, as compared with the initial aqueous extracts which are 100% derived from G. buchananii. The extract may be enriched to a similar degree in the concentration of flavonoid and other polyphenol compounds detected by spectroscopic methods.

The disclosure also provides fractions of the G. buchananii extract. In certain embodiments, these fractions are enriched with compounds that are effective in decreasing propulsive motility activity in the gastrointestinal tract and can be effective in treating various gastrointestinal disorders including diarrhea. These fractions can also be effective in inhibiting Ca²⁺ signaling in smooth muscle cells. In certain embodiments, these fractions and derivative compounds have antioxidant properties.

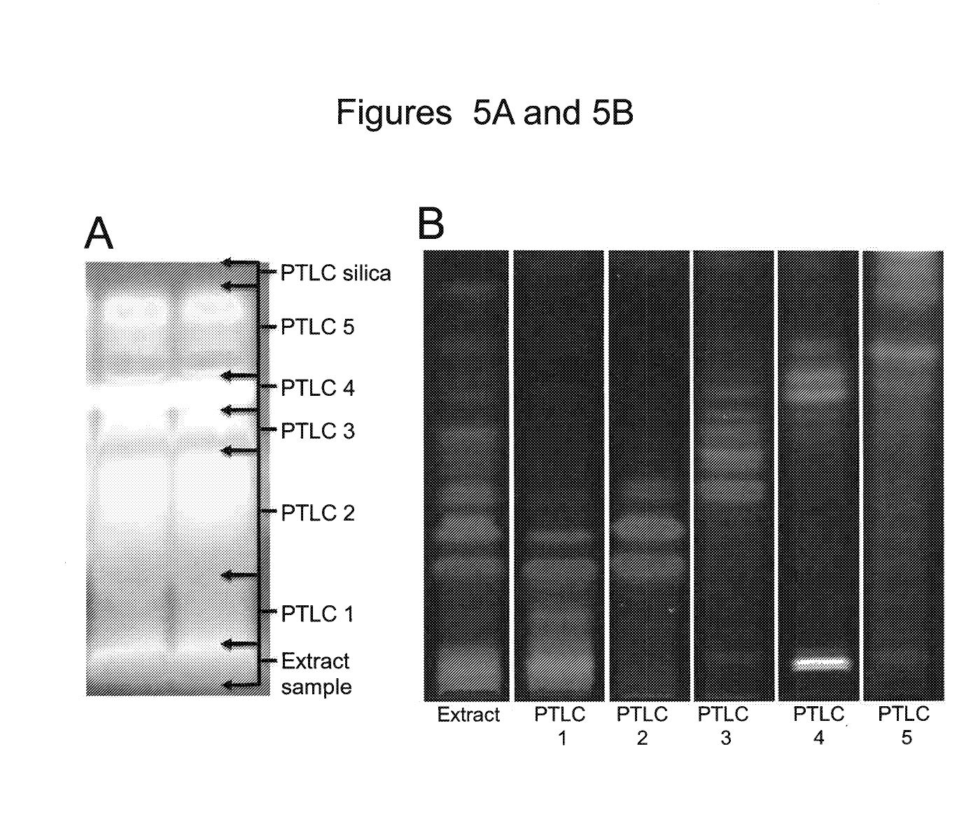

According to certain embodiments fractions are created using preparative thin layer chromatography (PTLC) and/or medium pressure liquid chromatography (MPLC) and/or high-performance liquid chromatography (HPLC). According to the disclosure, in some embodiments there are five fractions isolated by PTLC. Methods for isolating these PTLC fractions are found in the Examples and throughout the specification. These fractions can be defined by the methods used to isolate them. Further, these fractions can be defined by the coloration based on UV spectra as shown in FIGS. 5A and 5B. These fractions can also be defined by their antioxidative properties. In particular, PTLC fraction 1 (PTLC1) and PTLC fraction 5 (PTLC5) were particularly effective in inhibiting pellet velocity in diarrheic rats. Further, PTLC1 and PTLC5 increased appetite in diarrheic rats compared to control diarrheic rats. Lack of appetite is a symptom of diarrhea that exacerbates the loss of minerals through the stool. Increasing appetite in diarrheic patients would increase their ability to respond to the disease. PTLC1 and PTLC5 are also likely to reduce or prevent bloating which causes pain in subjects with diarrhea.

PTLC2 and PTLC3 increased pellet velocity in diarrheic rats. Thus, PTLC1 and PTLC5 contain compounds that are effective in decreasing pellet velocity and thus in treating diarrhea. PTLC2 and PTLC3 are effective in increasing pellet velocity and thus in treating constipation.

The compositions of PTLC1 and/or PTLC5 could be combined with various compounds to further decrease pellet velocity. One example is the 5-HT₄ receptor antagonist GR-113808. 5HT₃ receptor antagonists entirely inhibited the anti-motility actions of both fractions. Thus, it is possible that the compounds in PTLC1 and PTLC5 act through the 5HT₃ receptor. Methods for isolating these PTLC fractions are found in the Examples and throughout the specification. These fractions are defined by their antioxidative properties.

MPLC fractions include fractions M1-M8. Fractions 1-5 were characterized using 1D and 2D NMR techniques. These fractions can be defined as providing NMR output similar to that shown in the Examples and Figures below. These fractions can also be defined by the methods used to isolate them in the Examples below. Further, each of the fractions M1, M3, M4 and M5 contained a dominant compound of formulas 1-5 disclosed herein. These compounds are (2R,3R)-Taxifolin-6-C-β-D-glucopyranoside (1), and (2R,3R)-aromadendrin-6-C-β-D-glucopyranoside (2) both from M1; (2R,3R,2″R,3″R)-manniflavone (3) from M3; (2R,3R,2″R,3″R) GB-2 from M4 and (2R,3S,2″S) buchananiflavanone (5) from M5. These compounds have anti-oxidative properties. The properties of the compounds described above extend to any and all of their stereoisomers for example diastereomers, enantiomers and racemic mixtures of those enantiomers. These fractions can also be defined as being enriched in the compounds associated with them or with their relative anti-oxidative properties as defined in the Examples.

Fractions M4, M5 and M7 also showed the ability to decrease the motility of the gastrointestinal tract. Fraction M7 was subfractionated into fractions M7-1, M7-2, M7-3 and M7-4 as described below in the Examples. M7-4 also showed the ability to decrease the motility of the gastrointestinal tract. M4, M5 and M7 are also likely to reduce or prevent bloating which causes pain in subjects with diarrhea.

G. buchananii extract showed the ability to inhibit Ca²⁺ signaling in smooth muscle cells, as well as fraction M3 and (2R,3R,2″R,3″R)-manniflavone. Further, G. buchananii extract as well as fractions M4 and M5 and their compounds GB-2 and (2R,3S,2″S) buchananiflavanone have been shown to reduce pain. Fractions M6 and M7 have also shown promise in reduction of pain.

Combination Therapies

The compounds, compositions, extracts or fractions disclosed herein can be combined with other therapies appropriate for the treatment of gastrointestinal disorders or pain. Treatments for gastrointestinal disorders include oral rehydration salts, antibiotics, bismuth compounds, anti-motility agents and bile acid sequestrants. Treatments for pain include opiate and non-opiate pain relievers. Opiate pain relievers include morphine, diamorphine, heroin, buprenorphine, dipipanone, pethidine, dextromoramide, alfentanil, fentanyl, remifentanil, methadone, codeine, dihydrocodeine, tramadol, pentazocine, vicodin, oxycodone, hydrocodone, percocet, percodan, norco, dilaudid, darvocet or lorcet. Non-opiate pain relievers include salicylate, such as aspirin, amoxiprin, benorilate or diflunisal; an arylalkanoic acid, such as diclofenac, etodolac, indometacin, ketorolac, nabumetone, sulindac or tolmetin; a 2-arylpropionic acid (a “profen”), such as ibuprofen, carprofen, fenoprofen, flurbiprofen, loxoprofen, naproxen, tiaprofenic acid or suprofen; a fenamic acid, such as mefenamic acid or meclofenamic acid; a pyrazolidine derivative, such as phenylbutazone, azapropazone, metamizoie or oxyphenbutazone; a coxib, such as celecoxib, etoricoxib, lumiracoxib or parecoxib; an oxicam, such as piroxicam, lornoxicam, meloxicam or tenoxicam; or a sulfonanilide, such as nimesulide. Any of these compounds can be co-administered with any of the compositions, compounds, extracts or fractions disclosed herein.

These combination therapies can be provided in a single or multiple dosage forms. These dosage forms can be made into kits.

Pharmaceutical Compositions

The compounds described herein can be used in their final non-salt form. On the other hand, the present invention also encompasses the use of these compounds in the form of their pharmaceutically acceptable salts, which can be derived from various organic and inorganic acids and bases by procedures known in the art. Pharmaceutically acceptable salt forms of the compounds according to the invention are for the most part prepared by conventional methods. If the compound according to the invention contains a carboxyl group, one of its suitable salts can be formed by reacting the compound with a suitable base to give the corresponding base-addition salt. Such bases are, for example, alkali metal hydroxides, including potassium hydroxide, sodium hydroxide and lithium hydroxide; alkaline earth metal hydroxides, such as barium hydroxide and calcium hydroxide; alkali metal alkoxides, for example potassium ethoxide and sodium propoxide; and various organic bases, such as piperidine, diethanolamine and N-methylglutamine. The aluminium salts of the compounds are likewise included. In the case of certain compounds, acid-addition salts can be formed by treating these compounds with pharmaceutically acceptable organic and inorganic acids, for example hydrogen halides, such as hydrogen chloride, hydrogen bromide or hydrogen iodide, other mineral acids and corresponding salts thereof, such as sulfate, nitrate or phosphate and the like, and alkyl- and monoarylsulfonates, such as ethanesulfonate, toluenesulfonate and benzenesulfonate, and other organic acids and corresponding salts thereof, such as acetate, trifluoroacetate, tartrate, maleate, succinate, citrate, benzoate, salicylate, ascorbate and the like. Accordingly, pharmaceutically acceptable acid-addition salts of the compounds include the following: acetate, adipate, alginate, arginate, aspartate, benzoate, benzenesulfonate (besylate), bisulfate, bisulfite, bromide, butyrate, camphorate, camphorsulfonate, caprylate, chloride, chlorobenzoate, citrate, cyclopentanepropionate, digluconate, dihydrogenphosphate, dinitrobenzoate, dodecylsulfate, ethanesulfonate, fumarate, galacterate (from mucic acid), galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, isobutyrate, lactate, lactobionate, malate, maleate, malonate, mandelate, metaphosphate, methanesulfonate, methylbenzoate, monohydrogenphosphate, 2-naphthalenesulfonate, nicotinate, nitrate, oxalate, oleate, palmoate, pectinate, persulfate, phenylacetate, 3-phenylpropionate, phosphate, phosphonate, phthalate, but this does not represent a restriction.

Furthermore, the base salts of the compounds described herein include aluminum, ammonium, calcium, copper, iron (III), iron (II), lithium, magnesium, manganese(III), manganese(II), potassium, sodium and zinc salts, but this is not intended to represent a restriction. Of the above-mentioned salts, preference is given to ammonium; the alkali metal salts sodium and potassium, and the alkaline earth metal salts calcium and magnesium. Salts of the compounds which are derived from pharmaceutically acceptable organic non-toxic bases include salts of primary, secondary and tertiary amines, substituted amines, also including naturally occurring substituted amines, cyclic amines, and basic ion exchanger resins, for example arginine, betaine, caffeine, chloroprocaine, choline, N,N′-dibenzylethylenediamine (benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris(hydroxymethyl)methylamine (tromethamine), but this is not intended to represent a restriction.

The above-mentioned pharmaceutical salts which are preferred include acetate, trifluoroacetate, besylate, citrate, fumarate, gluconate, hemisuccinate, hippurate, hydrochloride, hydrobromide, isethionate, mandelate, meglumine, nitrate, oleate, phosphonate, pivalate, sodium phosphate, stearate, sulfate, sulfosalicylate, tartrate, thiomalate, tosylate and tromethamine, but this is not intended to represent a restriction.

The acid-addition salts of basic compounds are prepared by bringing the free base form into contact with a sufficient amount of the desired acid, causing the formation of the salt in a conventional manner. The free base can be regenerated by bringing the salt form into contact with a base and isolating the free base in a conventional manner. The free base forms differ in a certain respect from the corresponding salt forms thereof with respect to certain physical properties, such as solubility in polar solvents; for the purposes of the invention, however, the salts otherwise correspond to the respective free base forms thereof.

As mentioned, the pharmaceutically acceptable base-addition salts of the compounds are formed with metals or amines, such as alkali metals and alkaline earth metals or organic amines. Preferred metals are sodium, potassium, magnesium and calcium. Preferred organic amines are N,N′-dibenzylethylenediamine, chloroprocaine, choline, diethanolamine, ethylenediamine, N-methyl-D-glucamine and procaine.

The base-addition salts of acidic compounds according to the invention are prepared by bringing the free acid form into contact with a sufficient amount of the desired base, causing the formation of the salt in a conventional manner. The free acid can be regenerated by bringing the salt form into contact with an acid and isolating the free acid in a conventional manner. The free acid forms differ in a certain respect from the corresponding salt forms thereof with respect to certain physical properties, such as solubility in polar solvents; for the purposes of the invention, however, the salts otherwise correspond to the respective free acid forms thereof.

If a compound according to the invention contains more than one group which is capable of forming pharmaceutically acceptable salts of this type, the invention also encompasses multiple salts. Typical multiple salt forms include, for example, bitartrate, diacetate, difumarate, dimeglumine, diphosphate, disodium and trihydrochloride, but this is not intended to represent a restriction.

With regard to that stated above, it can be seen that the expression “pharmaceutically acceptable salt” in the present connection is taken to mean an active ingredient which comprises a compound in the form of one of its salts, in particular if this salt form imparts improved pharmacokinetic properties on the active ingredient compared with the free form of the active ingredient or any other salt form of the active ingredient used earlier. The pharmaceutically acceptable salt form of the active ingredient can also provide this active ingredient for the first time with a desired pharmacokinetic property which it did not have earlier and can even have a positive influence on the pharmacodynamics of this active ingredient with respect to its therapeutic efficacy in the body.

The disclosure furthermore relates to medicaments comprising at least one compound according to the invention and/or tautomers and stereoisomers thereof, including mixtures thereof in all ratios, and optionally excipients and/or adjuvants.

Pharmaceutical formulations can be administered in the form of dosage units which comprise a predetermined amount of active ingredient per dosage unit. Such a unit can comprise, for example, 0.05 mg to 1 g, preferably 1 mg to 700 mg, particularly preferably 5 mg to 100 mg, of a compound according to the invention, depending on the condition treated, the method of administration and the age, weight and condition of the patient, or pharmaceutical formulations can be administered in the form of dosage units which comprise a predetermined amount of active ingredient per dosage unit. Preferred dosage unit formulations are those which comprise a daily dose or part-dose, as indicated above, or a corresponding fraction thereof of an active ingredient. Furthermore, pharmaceutical formulations of this type can be prepared using a process which is generally known in the pharmaceutical art.

Pharmaceutical formulations can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such formulations can be prepared using all processes known in the pharmaceutical art by, for example, combining the active ingredient with the excipient(s) or adjuvant(s).

Pharmaceutical formulations adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the active-ingredient component can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or mannitol. A flavour, preservative, dispersant and dye may likewise be present.

Capsules are produced by preparing a powder mixture as described above and filling shaped gelatine shells therewith. Glidants and lubricants, such as, for example, highly disperse silicic acid, talc, magnesium stearate, calcium stearate or polyethylene glycol in solid form, can be added to the powder mixture before the filling operation. A disintegrant or solubiliser, such as, for example, agar-agar, calcium carbonate or sodium carbonate, may likewise be added in order to improve the availability of the medicament after the capsule has been taken.

In addition, if desired or necessary, suitable binders, lubricants and disintegrants as well as dyes can likewise be incorporated into the mixture. Suitable binders include starch, gelatine, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatine or polyvinylpyrrolidone, a dissolution retardant, such as, for example, paraffin, an absorption accelerator, such as, for example, a quaternary salt, and/or an absorbent, such as, for example, bentonite, kaolin or dicalcium phosphate. The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tabletting machine, giving lumps of non-uniform shape, which are broken up to form granules. The granules can be lubricated by addition of stearic acid, a stearate salt, talc or mineral oil in order to prevent sticking to the tablet casting moulds. The lubricated mixture is then pressed to give tablets. The compounds according to the invention can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups and elixirs, can be prepared in the form of dosage units so that a given quantity comprises a prespecified amount of the compound. Syrups can be prepared by dissolving the compound in an aqueous solution with a suitable flavour, while elixirs are prepared using a non-toxic alcoholic vehicle. Suspensions can be formulated by dispersion of the compound in a non-toxic vehicle. Solubilisers and emulsifiers, such as, for example, ethoxylated isostearyl alcohols and polyoxyethylene sorbitol ethers, preservatives, flavour additives, such as, for example, peppermint oil or natural sweeteners or saccharin, or other artificial sweeteners and the like, can likewise be added.

The dosage unit formulations for oral administration can, if desired, be encapsulated in microcapsules. The formulation can also be prepared in such a way that the release is extended or retarded, such as, for example, by coating or embedding of particulate material in polymers, wax and the like.

The compounds according to the invention and salts, solvates and physiologically functional derivatives thereof can also be administered in the form of liposome delivery systems, such as, for example, small unilamellar vesicles, large unilamellar vesicles and multilamellar vesicles. Liposomes can be formed from various phospholipids, such as, for example, cholesterol, stearylamine or phosphatidylcholines.

The compounds according to the invention and the salts, solvates and physiologically functional derivatives thereof can also be delivered using monoclonal antibodies as individual carriers to which the compound molecules are coupled. The compounds can also be coupled to soluble polymers as targeted medicament carriers. Such polymers may encompass polyvinylpyrrolidone, pyran copolymer, polyhydroxypropylmethacrylamidophenol, polyhydroxyethyl aspartamidophenol or polyethylene oxide polylysine, substituted by palmitoyl radicals. The compounds may furthermore be coupled to a class of biodegradable polymers which are suitable for achieving controlled release of a medicament, for example polylactic acid, poly-epsilon-caprolactone, polyhydroxybutyric acid, polyorthoesters, polyacetals, polydihydroxypyrans, polycyanoacrylates and crosslinked or amphipathic block copolymers of hydrogels.

Pharmaceutical formulations adapted for transdermal administration can be administered as independent plasters for extended, close contact with the epidermis of the recipient. Thus, for example, the active ingredient can be delivered from the plaster by iontophoresis, as described in general terms in Pharmaceutical Research, 3(6), 318 (1986).

Pharmaceutical compounds adapted for topical administration can be formulated as ointments, creams, suspensions, lotions, powders, solutions, pastes, gels, sprays, aerosols or oils.

For the treatment of the eye or other external tissue, for example mouth and skin, the formulations are preferably applied as topical ointment or cream. In the case of formulation to give an ointment, the active ingredient can be employed either with a paraffinic or a water-miscible cream base. Alternatively, the active ingredient can be formulated to give a cream with an oil-in-water cream base or a water-in-oil base.

Pharmaceutical formulations adapted for topical application to the eye include eye drops, in which the active ingredient is dissolved or suspended in a suitable carrier, in particular an aqueous solvent.

Pharmaceutical formulations adapted for topical application in the mouth encompass lozenges, pastilles and mouthwashes.

Pharmaceutical formulations adapted for rectal administration can be administered in the form of suppositories or enemas.

Pharmaceutical formulations adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose.

Suitable formulations for administration as nasal spray or nose drops with a liquid as carrier substance encompass active-ingredient solutions in water or oil.

Pharmaceutical formulations adapted for administration by inhalation encompass finely particulate dusts or mists, which can be generated by various types of pressurised dispensers with aerosols, nebulisers or insufflators.

Pharmaceutical formulations adapted for vaginal administration can be administered as pessaries, tampons, creams, gels, pastes, foams or spray formulations.

Pharmaceutical formulations adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multidose containers, for example sealed ampoules and vials, and stored in freeze-dried (lyophilised) state, so that only the addition of the sterile carrier liquid, for example water for injection purposes, immediately before use is necessary. Injection solutions and suspensions prepared in accordance with the recipe can be prepared from sterile powders, granules and tablets.

It goes without saying that, in addition to the above particularly mentioned constituents, the formulations may also comprise other agents usual in the art with respect to the particular type of formulation; thus, for example, formulations which are suitable for oral administration may comprise flavours.

A therapeutically effective amount of a compound according to the invention depends on a number of factors, including, for example, the age and weight of the animal, the precise condition that requires treatment, and its severity, the nature of the formulation and the method of administration, and is ultimately determined by the treating doctor or vet. However, an effective amount of a compound according to the invention for the treatment of neoplastic growth, for example colon or breast carcinoma, is generally in the range from 0.1 to 100 mg/kg of body weight of the recipient (mammal) per day and particularly typically in the range from 1 to 10 mg/kg of body weight per day. Thus, the actual amount per day for an adult mammal weighing 70 kg is usually between 70 and 700 mg, where this amount can be administered as a single dose per day or usually in a series of part-doses (such as, for example, two, three, four, five or six) per day, so that the total daily dose is the same. An effective amount of a salt or solvate or of a physiologically functional derivative thereof can be determined as the fraction of the effective amount of the compound according to the invention per se. It can be assumed that similar doses are suitable for the treatment of other conditions mentioned above.

The invention furthermore relates to medicaments comprising at least one compound according to the invention and/or tautomers and stereoisomers thereof, including mixtures thereof in all ratios, and at least one further medicament active ingredient.

The invention also relates to a set (kit) consisting of separate packs of (a) an effective amount of a compound according to the invention and/or tautomers and stereoisomers thereof, including mixtures thereof in all ratios, and (b) an effective amount of a further medicament active ingredient.

The set comprises suitable containers, such as boxes, individual bottles, bags or ampoules. The set may, for example, comprise separate ampoules, each containing an effective amount of a compound according to the invention and/or tautomers and stereoisomers thereof, including mixtures thereof in all ratios, and an effective amount of a further medicament active ingredient in dissolved or lyophilised form.

### DEFINITIONS

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only not intended to be limiting. Other features and advantages of the invention will be apparent from the following detailed description and claims.

For the purposes of promoting an understanding of the embodiments described herein, reference will be made to preferred embodiments and specific language will be used to describe the same. The terminology used herein is for the purpose of describing particular embodiments only, and is not intended to limit the scope of the present invention. As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic and/or pharmaceutical agents and equivalents thereof known to those skilled in the art, and so forth. Thus, for example, a reference to “a host cell” includes a plurality of such host cells, and a reference to “an antibody” is a reference to one or more antibodies and equivalents thereof known to those skilled in the art, and so forth.

“Treatment” as used herein refers to improving the symptoms of a given pathology or reducing the likelihood of the onset of symptoms. The pathologies referred to herein are gastrointestinal pathologies including diarrhea, colitis, ulcerative colitis, inflammatory bowel disorder, irritable bowel syndrome, ischemic bowel disease, Crohn's disease, colitis due to acute and chronic intestinal ischemia, hormone-secreting tumors, ulcerative colitis, celiac disease, Whipple disease, Graft-versus-Host disease after stem cell transplantation, food poisoning, dysentery, viral gastroenteritis, food allergy, food intolerance, bile acid malabsorption and infection. Infection can be caused by certain strains of E. coli, norovirus, rotavirus, adenovirus (types 40 and 41), astroviruses, Campylobacter species, particularly jejuni, Salmonella, Shigella, Giardia, Cholera, Clostridium difficile, Staphylococcus aureus, Entamoeba histolytica, Cryptosporidium. In certain embodiments, the pathology is diarrhea. Symptoms associated with diarrhea include increase in frequency of bowel movements, loose stool, bloating, lack of appetite, increased secretion of fluid into the intestine, increased speed of passage of stool through the intestine, dehydration, mineral abnormalities, gastrointestinal pain and anal irritation. In certain embodiments, compounds, compositions extracts and fractions of extracts disclosed herein can reduce the symptoms of diarrhea by reducing speed of passage of stool through the intestines and/or reduction in gastrointestinal pain. However, compounds, compositions extracts and fractions of extracts disclosed herein can improve any symptom or reduce the likelihood of onset of symptoms associated with diarrhea.

As used herein, an “effective amount” can refer to a “therapeutically effective amount” or a “biologically effective amount.”

A “therapeutically effective amount” is an amount of a compound, composition, extract or fraction disclosed herein that when administered to a subject results in treatment of the subject. The dosage administered, as single or multiple doses, to an individual may vary depending upon a variety of factors, including the route of administration, patient conditions and characteristics (sex, age, body weight, health, size), extent of symptoms, concurrent treatments, frequency of treatment and the effect desired. Adjustment and manipulation of established dosage ranges are well within the ability of those skilled in the art, as well as in vitro and in vivo methods of determining the dosage of the compounds, extracts and fractions disclosed herein.

A “biologically effective amount” is an amount of a compound, composition, extract or fraction disclosed herein that when administered to a biological sample results in a biologically relevant effect. For example, this effect could be anti-oxidation (reduction or reduced oxidation of a biological system) decrease in Ca²⁺ signaling in smooth muscle cells, reduction or increase in HT signaling, reduction or increase in gastrointestinal motility either in vivo or in an in vitro model using cultured cells, tissues or organs or reduction or increase in nociceptor function.

### EXAMPLES

It is understood that modifications which do not substantially affect the activity of the various embodiments of this invention are also provided within the definition of the invention provided herein. Accordingly, the following examples are intended to illustrate but not limit the present invention. While the claimed invention has been described in detail and with reference to specific embodiments thereof, it will be apparent to one of ordinary skill in the art that various changes and modifications can be made to the claimed invention without departing from the spirit and scope thereof. Thus, for example, those skilled in the art will recognize, or be able to ascertain, using no more than routine experimentation, numerous equivalents to the specific substances and procedures described herein. Such equivalents are considered to be within the scope of this invention, and are covered by the following claims.

### Example 1

### The Traditional Antidiarrheal Remedy, Garcinia buchananii Stem Bark Extract, Inhibits Propulsive Motility and Fast Synaptic Potentials in the Guinea Pig Distal Colon

The aim of this study was to determine the effects of an aqueous G. buchananii stem bark extract on peristalsis and neurotransmission activity of the guinea pig distal colon. The mechanisms and efficacy of G. buchananii bark extract using the guinea pig distal colon model of gastrointestinal motility was investigated.

Methods:

Stem bark was collected from G. buchananii trees in their natural habitat of Karagwe, Tanzania. Bark was sun dried and ground into fine powder, and suspended in Krebs to obtain an aqueous extract. Isolated guinea pig distal colon was used to determine the effect of the G. buchananii bark extract on fecal pellet propulsion. Intracellular recording was used to evaluate the extract action on evoked fast excitatory postsynaptic potentials (fEPSPs) in S-neurons of the myenteric plexus.

Key Results:

Garcinia buchananii bark extract inhibited pellet propulsion in a concentration-dependent manner, with an optimal concentration of −10 mg powder per mL Krebs. Interestingly, washout of the extract resulted in an increase in pellet propulsion to a level above basal activity. The extract reversibly reduced the amplitude of evoked fEPSPs in myenteric neurons. The extract's inhibitory action on propulsive motility and fEPSPs was not affected by the opioid receptor antagonist, naloxone, or the alpha-2 adrenoceptor antagonist, yohimbine. The extract inhibited pellet motility in the presence of gammaaminobutyric acid (GABA), GABA_{A} and GABA_{B} receptor antagonists picrotoxin and phaclofen, respectively. However, phaclofen and picrotoxin inhibited recovery rebound of motility during washout.

Materials and Methods

Animals and Solutions:

Male adult guinea pigs (Charles River, Montreal, Canada and Elm Hill Breeding Labs, Chelmsford, Mass., USA) weighing 250-350 g were housed in metal cages with soft bedding. Animals had access to food and water ad libitum and were maintained at 23-24° C. on a 12:12 h light-dark cycle. Animals were anesthetized with isoflurane and exsanguinated. The entire distal colon was removed, stored in Krebs solution (mmol L⁻¹: NaCl, 121; KCl, 5.9; CaCl₂, 2.5; MgCl₂, 1.2; NaHCO₃, 25; NaH₂PO₄, 1.2; glucose, 8; aerated with 95% O₂/5% CO₂; all from Sigma-Aldrich, St. Louis, Mo., USA) and used for subsequent motility or electrophysiology experimentation. The Institutional Animal Care and Use Committees of both the University of Idaho and the University of Vermont approved all animal procedures.

Preparation of Aqueous Garcinia buchananii Extract:

Stem bark samples were collected from stems of G. buchananii trees (family name Clusiaceae; vernacular name Omusharazi; in their natural habitat in Nyakasimbi village (GPS: Latitudes: −1.852247, Longitudes 31.024017; altitude: ˜1600 m), Karagwe, Kagera, Tanzania. The bark specimens used are similar to G. buchananii bark deposited at the Department of Botany herbarium, University of Dar es Salaam (voucher specimen # DK 063/06, see Kisangau, D. P. and others, 2007). Bark was collected in October 2006 and September 2009, trimmed into small pieces, sun dried for 1 week and ground into powder using a wooden mortar and pestle. The powder was sieved using 1-mm mesh with criss-cross patterning. The powder was transported using airtight bags, and was stored at 4° C. while protected from light. Freshly diluted extract solutions were used for each experiment. Appropriate amounts of the powder were weighed, mixed with 100 mL Krebs, and stirred for 30 min at room temperature. The mixtures were filtered using analytical filter paper [Schleicher and Schuell Blue Ribbon filter paper 589/3 (General Lab. Supplies, Pasadena, Tex., USA); 0.2 lm retention] and the filtrate (G. buchananii bark extract) was used as indicated in the procedures.

Gastrointestinal Motility Assays: Segments (˜10 cm long) of distal colon were pinned on either end in a Sylgard-lined 50 mL organ bath, continuously perfused with oxygenated Krebs solution (rate: 10 mL min⁻¹) and maintained at temperatures between 36 and 37° C. Tissues were initially allowed to equilibrate for 30 mins in re-circulating Krebs solution. Colonic motility was studied using the Gastronintestinal Motility Monitoring system (GIMM; Med-Associates Inc., Saint Albans, Vt., USA), which included a digital video camera (Catamount Research and Development Inc, St Albans, Vt., USA) to film fecal pellet propulsion. Trials were separated by 5-min recovery periods between successive pellet runs. The GIMM system software calculated velocities by tracking pellets as they traversed colon segments. After initial equilibration, five pellet propulsion trials in vehicle solution were obtained to determine the basal velocity for each tissue preparation. To reduce variations between runs and experiments, the values obtained were used to generate a normalized basal velocity by dividing the values of the 4th-5th runs by the value of the 1st-2nd runs. Garcinia buchananii stem bark extract and test compounds dissolved in 100 mL of Krebs solution were superfused either into the organ bath or into the lumen of the colon using polyethylene tubing [PE 205; outside diameter 9.5 mm (BD-Worldwide, Sparks, Md., USA)]. The effects of extract and test compounds were evaluated by obtaining velocities of four trials taken at 5-min intervals (5-20 min) Normalized velocity data for these treatments were obtained by dividing the test velocities acquired by the average velocity of the five pellet propulsion runs performed in vehicle solution (runs 1-5, above). Normalized data became ratios without units, and were presented as a percent velocity of basal activity for each tissue. Velocities of pellet propulsion were also studied during washout of tissue with Krebs solution (after extract or test compound application) for 20 min

Intracellular Recording: To study fast excitatory postsynaptic potentials, longitudinal muscle-myenteric plexus preparations of guinea pig colon were pinned and stretched in a 2.5 mL Sylgard-lined recording chamber. The tissues were maintained at 36-37° C. by continuous perfusion with re-circulating oxygenated Krebs solution (10 mL min⁻¹). Nifedipine (5 μmol L⁻¹) and atropine (200 nmol L⁻¹) were added to Krebs solution to limit muscle contractions. Myenteric ganglia were visualized at ×200 using Hoffman modulation contrast optics on an inverted microscope (Nikon Diaphot, Melvilee, N.Y., USA).

Individual neurons were randomly impaled using glass microelectrodes, which were filled to the shoulder with 1.0 mol L⁻¹ KCl and topped off with 2.0 mol L⁻¹ KCl, generating a range of 50-150 MΩ input resistance. Membrane potential was measured with an Axoclamp-2A amplifier (Axon Instruments, Union City, Calif., USA) and the electrical signals were acquired and analyzed using PowerLab Chart version 5.01 (ADlnstruments, Castle Hills, Australia).

Input resistance and resting membrane potential were determined for neurons before and after exposure to G. buchananii extract. Using monopolar extracellular electrodes made from Teflon®-insulated platinum wire, synaptic input to myenteric neurons was elicited by direct single pulse stimuli (0.5 ms duration) applied to interganglionic fiber tracts. S-type neurons were identified based on the existence of fast excitatory postsynaptic potentials (fEPSPs) and the lack of a shoulder on the re-polarizing phase of the action potential. Amplitudes of the maximum fEPSPs were acquired while injecting hyperpolarizing currents to maintain membrane potential (˜−90 MV) and avoid action potentials. The amplitude was measured by taking the difference in voltage between the peak of each fEPSP and the membrane holding potential. Only neurons with an input resistance >50 MV were considered to be healthy and selected for study.

Data Analysis: Statistical analysis of data was done using Student's t-test, oneway ANOVA, and the Newman-Keul's multiple comparison post-test using GraphPad Prism 4 software (GraphPad Software Inc., San Diego, Calif., USA). Data were expressed as the mean±SEM for n values representing the number of colon segments from different animals used in each experiment. Statistical differences were considered significant at a P-value <0.05. Drugs and other materials Phaclofen, picrotoxin, yohimbine hydrochloride, GABA (c-aminobutyric acid), nifedipine, atropine, and naloxone hydrochloride were purchased from Sigma-Aldrich Chemical (St. Louis, Mo., USA).

Results

Intraluminal VS Serosal Application of Garcinia buchananii Bark Extract and Rapid Reversal of Induced Inhibition.

Control Velocity:

Average basal velocity of pellet propulsion was 2.4±0.6 mm s⁻¹ (n=28). The normalized basal pellet velocity computed by dividing the values of pellet velocities of the 4th-5th runs by the value of the 1st-2nd runs, with the ratio expressed as 100% was: ˜99.6%, ±2.0%; n=18.

Effect of Garcinia buchananii Extract on Basal Pellet Motility.

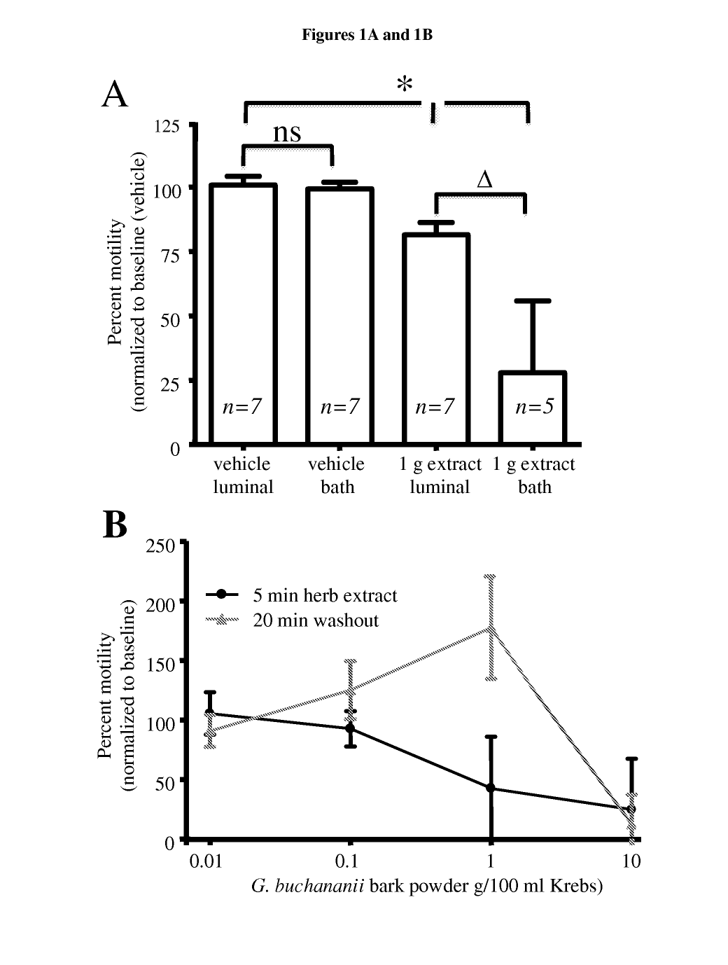

Aqueous extract from G. buchananii bark powder did not alter the pH of Krebs (vehicle solutions). When added to the bathing solution, the extract rapidly inhibited pellet propulsion in a concentration dependent manner (FIG. 1A, B). The onset of a response occurred within 1 min of exposure. The extract caused a less extensive reduction in pellet propulsion when administered to the colon intraluminally (1 g bark powder/100 mL extract intraluminal: 80.9%±6.3%, n=7 VS 1 g bark powder/100 mL extract bath: 27. 8%±27.8%; n=5; 10 min; P<0.001; FIG. 1A). The extract-induced inhibition of propulsive motility was rapidly reversed by washout, which increased pellet velocity by 30-100% above basal activity with exception of an extract prepared using 10 g bark powder/100 mL Krebs (FIG. 1B).

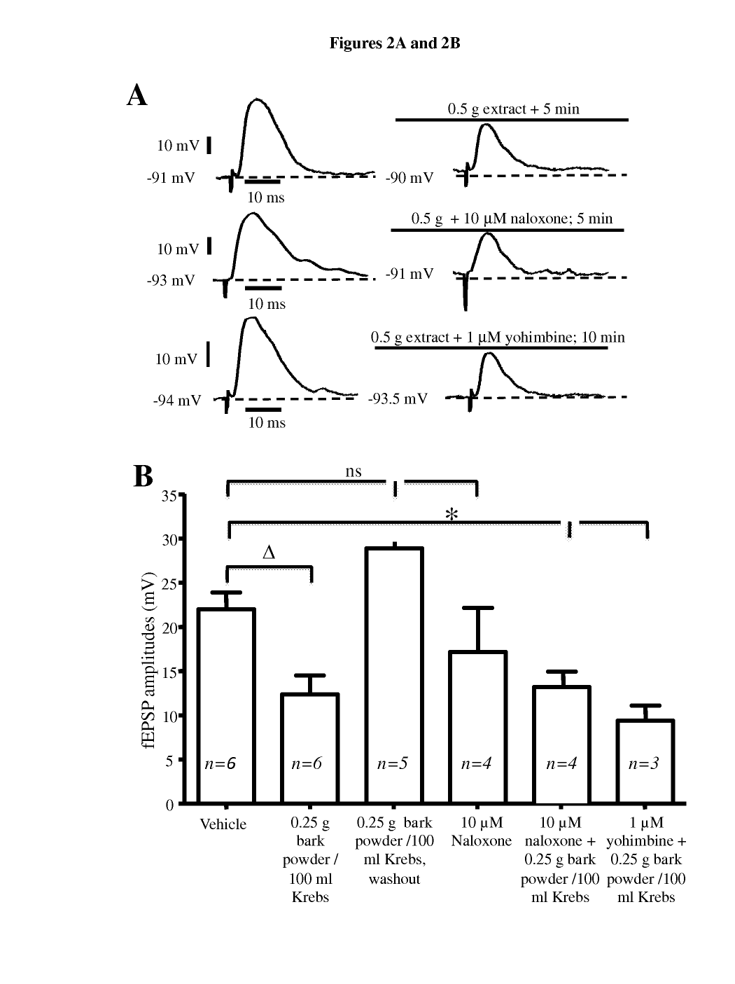

Garcinia buchananii extract inhibits enteric neurotransmission. Intracellular recording was used to study the effect of G. buchananii extract on 24 S-neurons in the myenteric ganglia of guinea pig distal colon using longitudinal smooth muscle-myenteric plexus preparations (FIG. 2A, B). S-type neurons exhibit fEPSPs after application of single electrical stimuli to interganglionic nerve fiber tracts. We found that compared with the motility assay, lower concentrations of G. buchananii extract (0.25-0.5 g bark powder/100 mL Krebs) caused a considerable decrease of fEPSP amplitudes. Application of G. buchananii extract (0.25 g bark powder/100 mL Krebs) did not alter the resting membrane potential (RMP; vehicle: −52.08±0.8, n=6 VS −50.78±0.6 n=4, P=0.33) or input resistance (vehicle: 111.2±14.3, n=6 VS 97.7±11.5, n=3; P=0.92) of S-neurons.

Application of the extract in the presence of naloxone (n=4) and yohimbine (n=3) did not alter the membrane potential of S-type myenteric neurons. Changes in neuronal excitability, in terms of rheobase or presence of anodal break action potentials, were not observed. When evoked synaptic potentials were evaluated, the extract (0.25-0.5 g bark powder/100 mL Krebs) reduced fEPSP amplitudes of S-neurons after 2-5 mins of application (vehicle: 21.9±1.9 MV, n=6 VS 0.25 g bark powder/100 mL Krebs: 12.3±2.1 MV, n=6; P=0.007; 5-min interval; FIG. 2A, B). The effect of the extract on fEPSP amplitudes was readily reversed by washout (0.25 g bark powder/100 mL Krebs: 13.4±1.2 MV VS 10-min washout: 28.8±2.08 MV; n=5; P=0.007; FIG. 2B). The amplitude of fEPSPs in S-neurons rebounded to slightly above basal level following washout (vehicle: 21.9±1.9 MV, n=13 VS 10-min washout: 28.8±2.08 MV, n=5; P=0.06).

The Effects of Garcinia buchananii Extract are not Altered by Opioid or Alpha-2 Adrenergic Receptor Antagonists.

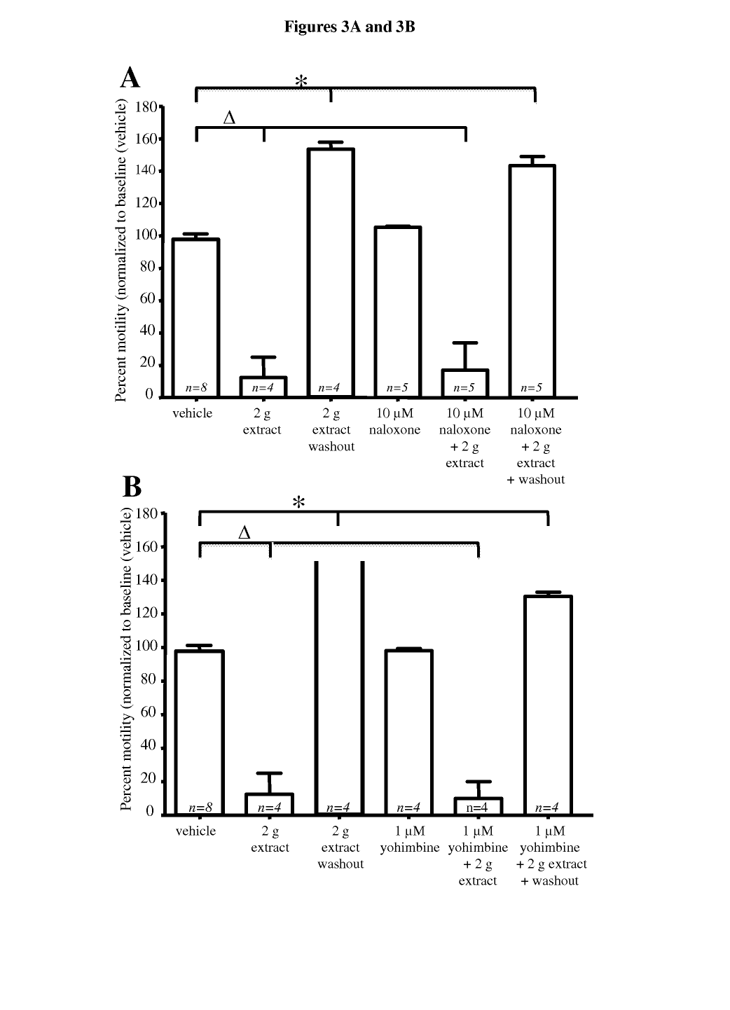

Previous studies have demonstrated that opioid and alpha-2 adrenergic receptor agonists result in inhibition of enteric ganglia fEPSPs. To determine whether G. buchananii extract acts via activation of these receptors, the actions of the extract was tested in the presence of receptor antagonists. The μ-opiod receptor antagonist naloxone (10 μmol L⁻¹) and alpha-2 (2a, 2b, 2c)-adrenergic receptor antagonist yohimbine (1 μmol L⁻¹) had no effect on fEPSPs. Application of the extract in the presence of naloxone (vehicle: 21.9±1.9 MV, n=6 VS 0.25 g bark powder/100 mL Krebs+10 μmol L⁻¹ naloxone: 13.1±1.7 MV; n=4; P=0.03) and yohimbine (vehicle: 21.9±1.9 MV, n=6 VS 0.25 g bark powder/100 mL Krebs+1 μmol L⁻¹ yohimbine: 9.3±1.7 MV, n=3; P=0.009) did not alter the inhibitory actions of the extract on fEPSPs (FIG. 2B). In motility studies, G. buchananii extract 2 g bark powder/100 mL Krebs rapidly reduced pellet velocity (vehicle: 99.7%±3.7%, n=8 VS 2 g bark powder/100 mL Krebs extract: 12.5%±12.0%, n=4; P<0.001; 10 min; FIG. 3A, B). Like in fEPSP studies, motility assays showed no change in the ability of the G. buchananii bark extract to reduce pellet propulsion while in the presence of naloxone (10 μmol L⁻¹) or yohimbine (1 μmol L⁻¹) (vehicle: 99.7%±3.7%, n=8 VS 2 g bark powder/100 mL Krebs extract+10 μmol L⁻¹ naloxone: 20.4%±20.4%; n=5; P<0.001; vehicle: 99.7%±3.7%, n=8 VS 2 g bark powder/100 mL Krebs extract+1 μmol L⁻¹ yohimbine: 10.0%±10.0%; n=6; 10-min interval; P<0.001; FIG. 3A, B).

Garcinia buchananii Bark Extract Inhibits GABA Receptors

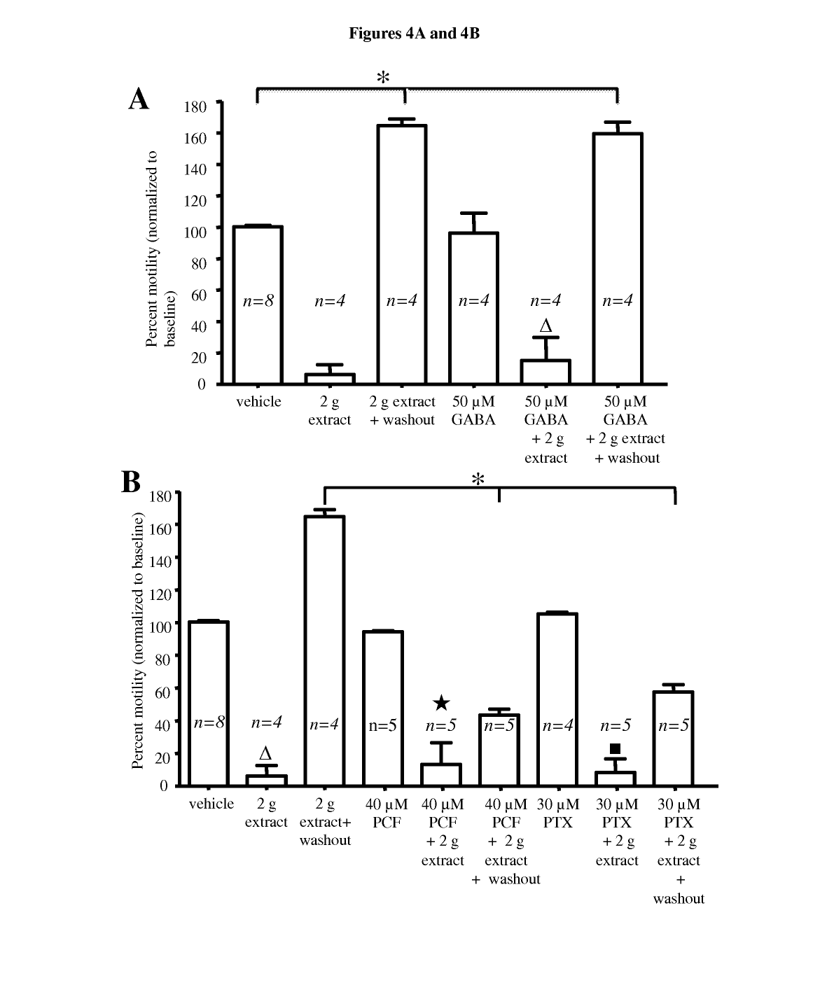

Flavonoids, which have been found in the bark of Garcinia plants, inhibit neurotransmission in the brain by acting via gamma-aminobutyric acid (GABA) receptors. Colonic motility assays were used to examine whether G. buchananii extract interacts with GABA receptors. Garcinia buchananii extract (2 g bark powder/100 mL Krebs) inhibited pellet propulsion in the presence of GABA agonist GABA (50 μmol⁻¹; FIG. 4A), the GABA_{A}-antagonist picrotoxin (PTX, 30 μmol L⁻¹) and the GABA_{B}-antagonist phaclofen (PCF, 40 μmol L⁻¹), respectively (FIG. 4B). While GABA showed no influence on recovery of pellet propulsion from the inhibitory effects of the extract, both phaclofen and picrotoxin caused a slower rate of recovery, such that normal propulsive activity was not achieved after 20 mins of Krebs washout (2-g extract washout: 157.0%±6.0%, n=4 VS 2-g extract+PCF washout: 43.5%±21.4%; n=5; P<0.001 and 2-g extract+PTX washout: 58.5%±8.1%; n=5; P<0.001; FIG. 4A, B).

Discussion

The purpose of this study was to determine whether G. buchananii extract, a herbal remedy for diarrheal diseases in sub-Saharan Africa, inhibits colonic motility, and to begin to identify the target tissues and the cellular mechanisms underlying these effects. We found that G. buchananii extract contains readily soluble bioactive components that rapidly inhibit propulsive motility in guinea pig distal colon segments.

This inhibitory activity occurs via activation of yet to be identified targets in the mucosa and myenteric ganglia. The extract's action involves an inhibition of synaptic transmission presumably by either reducing presynaptic neurotransmitter release, or by a blockade of postsynaptic excitatory responses in the myenteric ganglia. The motility and synaptic inhibitory actions were not mediated by the activation of presynaptic opioid or α-adrenergic receptors, which are the targets of most antimotilic drugs commonly used to treat diarrheal diseases.

The findings of this study demonstrated that the components of the G. buchananii bark extract inhibit motility from both the mucosal and serosal surfaces of the guinea pig colon. Although an antimotility response with a higher magnitude was obtained by applying the extract on the serosal surface, the onset of responses in both cases did not differ, occurring as early as 1 min after exposure to the extract. The variation between the two routes suggests that when G. buchananii bark extract was applied to the bathing solution, bioactive components activated a larger subset of myenteric neurons, as this bath application exposed the serosal surface to the extract. Consequently the myenteric plexus was rapidly exposed to a larger extract volume.

The findings also suggested a reduced rate of target site access following oral administration. These ideas are supported by the findings that lower concentrations of the bark extract are required to inhibit fEPSPs in longitudinal smooth muscle-myenteric preparations VS higher concentrations need for bath and intraluminal application in motility studies using intact colon segments. In either case, the responses appear to be mainly a result of inhibition of neurotransmission in the myenteric plexus.

Interestingly, at concentrations of less than 10 g G. buchananii bark powder per 100 mL Krebs, the inhibition of propulsion was reversed after 10-20 min washout. Upon washout, the velocities rebounded beyond the basal level by up to 70%. This suggests that G. buchananii extract may contain pro-motility components. This idea is supported by observations that G. buchananii bark extract can be separated into fractions of antimotilic and pro-kinetic activities using thin layer chromatography and HPLC (demonstrated by M6 and M8 in this application). Several other possibilities for these observations exist. Differences in the activation rates of cellular processes needed for inhibition/stimulation of propulsion and tissue metabolism of prokinetic VS antimotilic components are proposed explanations. The findings support the concept that mild overdose following consumption of G. buchananii extract can readily be reversed.

One major action of G. buchananii bark extract reported here was inhibition of interneuronal transmission in the myenteric ganglia. Fast excitatory synaptic transmission in the ENS plays a critical role of regulating intestinal motility. Basal resting membrane potentials, input resistance, and fEPSP amplitudes observed in this study correspond with previous findings in the myenteric ganglia of guinea pig distal colon. One theory based upon the findings reported here, is that G. buchananii bark extract contains bioactive components that either inhibit presynaptic neurotransmitter release from myenteric nerve terminals, or interfere with postsynaptic responses. Furthermore, this study has demonstrated that inhibition was not mediated by activation of presynaptic opioid or α-2 adrenergic receptors, which are the targets of some antidiarrheal drugs. In the guinea pig distal colon, myenteric neurons exhibit mixed fEPSPs with a larger proportion being regulated by nicotinic acetylcholine receptors. It has been proposed that plant polyphenols can selectively block nicotinic acetylcholine receptors, showing another potential mechanism for G. buchananii bark extract to inhibit fEPSPs.

Presynaptic inhibition of neurotransmission in the ENS can involve activation of α2-adrenoceptors, opioid receptors, muscarinic M₂ receptors, Adenosine A₁, and 5-HT_{1A} receptors, all of which couple to G-proteins and eventually modulate neurotransmitter release.

The bioactive components in G. buchananii bark extract appear to affect enteric neurotransmission at least in part via GABA receptors. Recent studies using botanical extracts suggest that bioflavonoids inhibit neurotransmission in the brain. These botanical metabolites either inhibit or activate GABA receptors. There is also evidence suggesting that flavonoids and xanthones constitute the major components of some Garcinia bark extracts. GABA is a neurotransmitter found in interneurons, which mediates activation of cholinergic excitatory neurons of the guinea pig distal colon. In the present study, GAB_{A} did not affect G. buchananii bark extract's activity. However, GABA_{A} and GABA_{B} receptor antagonists suppressed the rebound of propulsive activity during washout, suggesting that if rebound is due to activity of pro-kinetic components, they act as ligands of GABA_{A} and GABA_{B} receptors. It is likely that these pro-kinetic components cannot readily be washed out. In light of these findings, further studies are needed to distinguish how the components of the extract affect GABA neurotransmission in the ENS.

Within our model, it is likely that bioactive components of G. buchananii bark extract are acting via the colonic mucosa to inhibit peristaltic activity, and the specific mucosal target receptors are not known. The antimotility effects are elicited rapidly. Dissolving the bark powder into Krebs solution seems to effectively incorporate the bioactive components of G. buchananii bark into vehicle solution. Botanical extracts and their derivatives are decomposed and extensively metabolized or modified in the GI tract. It is unclear whether the components that inhibit colonic motility in this study would reach the colon following oral administration, or whether it is the systemically delivered metabolic derivatives that act as antidiarrheal remedies.

Taken together, these findings indicated that G. buchananii bark extract was capable of acting via mucosal targets and/or myenteric S-type neurons to inhibit propulsive motility in guinea pig distal colon Inhibition of synaptic transmission is likely to reduce bowel pain and discomfort, often associated as symptoms of diarrhea. Garcinia plant extracts contain components with anti-inflammatory, antiprotozoa, antibacterial, antiviral, and antioxidant activity. These characteristics and our findings suggest the bark of G. buchananii is promising to become an effective antidiarrheal botanical remedy.

In conclusion, our findings suggest that G. buchananii bark extract contains readily soluble bioactive compounds that act via mucosal targets or directly on the ENS to reduce propulsive motility through inhibition of S-neuron synaptic activity.

### Example 2

### Garcinia buchananii Bark Extract is an Effective Anti-Diarrheal Remedy

Oral rehydration therapy (ORT) is a recommended method of treatment for diarrhea. However, one disadvantage of ORT is that it does not reduce pain and shorten the duration of diarrheal symptoms. Garcinia buchananii bark extract is a non-opiate anti-diarrheal preparation traditionally used in parts of Africa to treat various diarrheal diseases and associated abdominal discomfort and pain. However, little or nothing is known about the efficacy, mechanism of action or the safety of the bark extract, and the bioactive components of the bark have not been characterized. The aim of our study was to show that G. buchananii bark extract is effective in stopping diarrhea and to determine the biologically active components in the extract. Methods: A high-lactose diet (35% of the total nutritional content) was used to induce diarrhea in 10 week old Wistar rats (389.2+/−6.3 g). Diarrheic rats were treated using G. buchananii bark extract (0.1 g, 0.5 g, 1 g and 5 g), and with the anti-diarrheal drug, loperamide (8.4 mg), added to 1 L drinking water. Rats were monitored for changes in consistency of pellets and stool mass, fecal fluid and urine production (mass; g and volume; mL). Rats were considered diarrheic if they produced watery stools, soft, yellowish stools compared to normal, pliable, soft, well-formed pellets as previously described by other researchers. Preparative thin layer chromatography (PTLC) was used to define, separate and isolate fractions of the bark extract based on color indexes (FIG. 5A). The effect of the isolated fractions on bowel motility was studied using fecal pellet propulsion assays in isolated guinea pig distal colon. Phytochemical analyses were conducted on the extract.

Results:

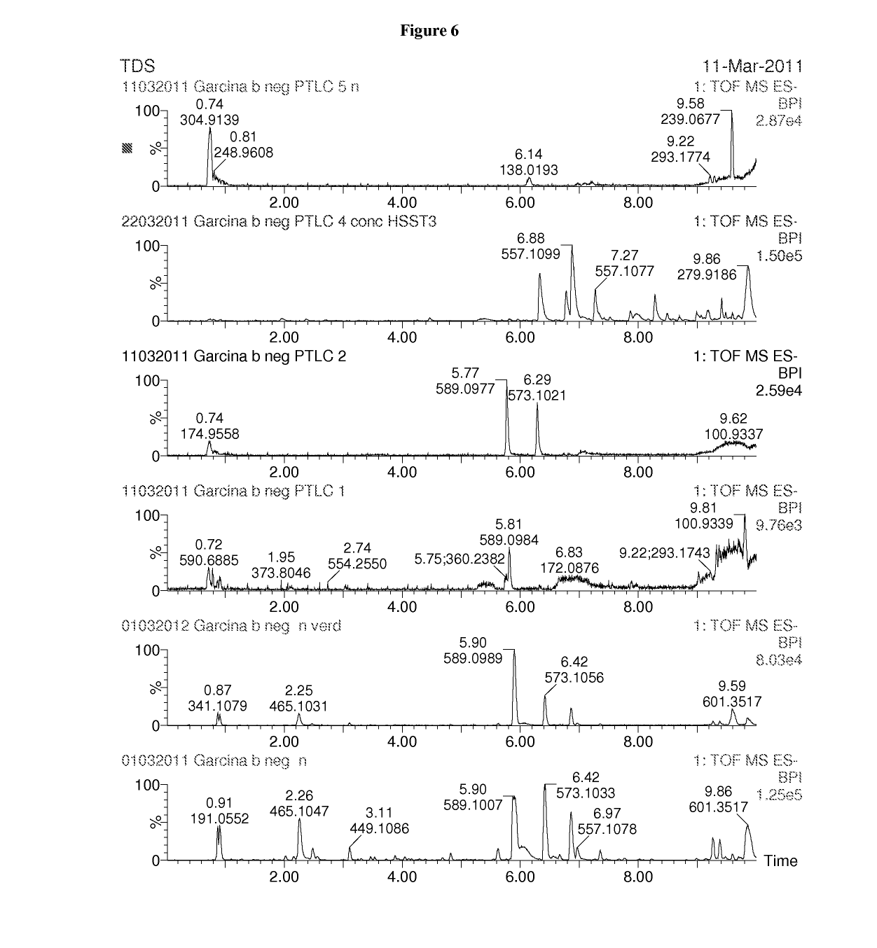

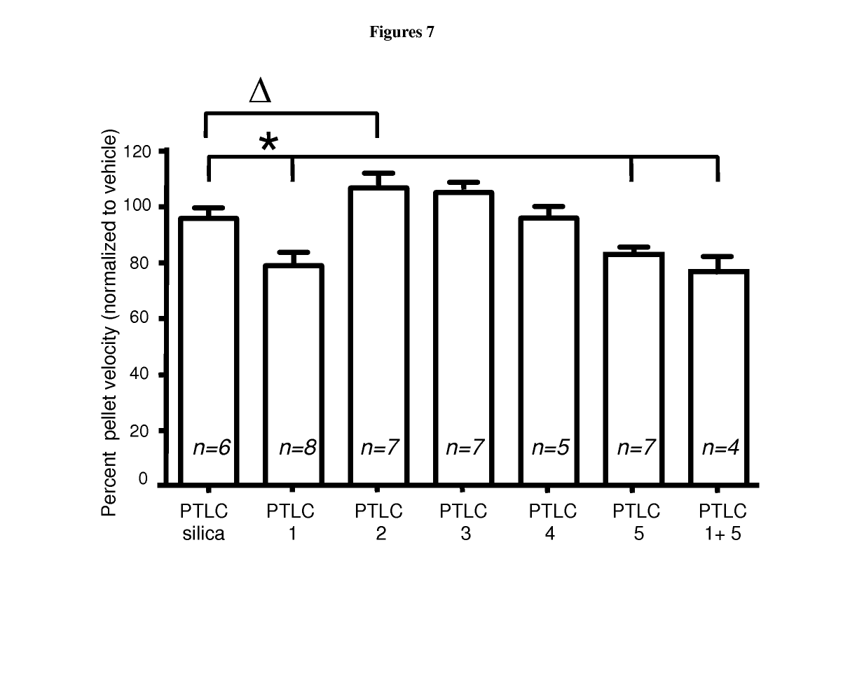

Five fractions were isolated using PTLC. These fractions were labeled as PTLC 1-5 (FIG. 5A). Each of these fractions can be isolated using these methods and isolating the fractions shown in FIG. 5A. Further separation of PTLC1-5 fractions using high performance thin layer chromatography (FIG. 5B) and deeper analysis of PTLC1-5 using UPLC-TOF-MS (FIG. 6) showed that each PTLC fraction contained a minimum of three distinct compounds. PTLC 1 and PTLC 5 inhibited pellet velocity, PTLC2 and PTLC3 increased pellet velocity, and PTLC 4 had no effect (FIG. 7). Flavonoids and sugars were detected in both fractions, phenols were found only in PTLC1, whereas alkaloids, tannins, and steroids were exclusively found in PTLC5. PTLC2, 3, and 4 were not subjected to chemical testing, as they did not have anti-motility effects. Diarrheic rats passed watery, loose feces, and were less active and alert than healthy rats. Rats treated with 0.1 g G. buchananii recovered from diarrhea, gained weight, and consumed more water and food as compared to the other groups of rats. However, rats treated with 1 g, 5 g extract, and with loperamide recovered rapidly from their diarrheic condition, as indicated by the hardening of their fecal pellets. Interestingly, rats treated with 1 g of G. buchananii bark extract, PTLC 1 (45 mg/300 ml) and PTLC 5 (11.5 mg/300 ml) produced more fecal pellets than rats treated with loperamide and 5 g extract.

G. buchananii is an effective anti-diarrheal remedy having an efficacy comparable to that of loperamide. Taken together our results suggest that G. buchananii bark extract contains anti-motility and pro-kinetic compounds with potential for diarrhea and constipation treatment.

### Example 3

### Garcinia buchananii Bark Extract Reduces High-Threshold Colonic Afferent Mechano-Sensitivity and Reverses Acute Inflammatory Mechanical Hypersensitivity

Garcinia buchananii bark extract is a traditional African remedy for diarrhea, dysentery, abdominal discomfort and pain. It has recently been shown that this extract reversibly reduces the amplitude of evoked fast excitatory post-synaptic potentials in myenteric neurons and correspondingly reduces colonic propulsive motility. However, it is currently unclear how this extract works to relieve abdominal discomfort and pain. As such we determined the effect of Garcinia buchananii bark extract on mechanosensitive afferents innervating the colon.

Methods:

An in vitro mouse colon preparation was used to study the mechano- and chemo-sensory function of serosal and mesenteric splanchnic afferents. These afferents have high mechanical activation thresholds to distension (>35 mmHg), circular stretch (>8 g) and probing with von Frey hairs. Mechanical hypersensitivity was induced by rectal administration of 0.1 mL (130 μg/mL) TNBS. Afferents were studied in healthy mice or mice with inflammation (7 days post-TNBS). 0.5 g of powdered Garcinia buchananii bark was added to 100 mL of Krebs solution, stirred for 30 mins and filtered to obtain an extract. This extract was applied for 5 mins to receptive fields via a small chamber on the colonic mucosal surface and the effects on mechanical sensitivity and direct excitation were measured.

Results:

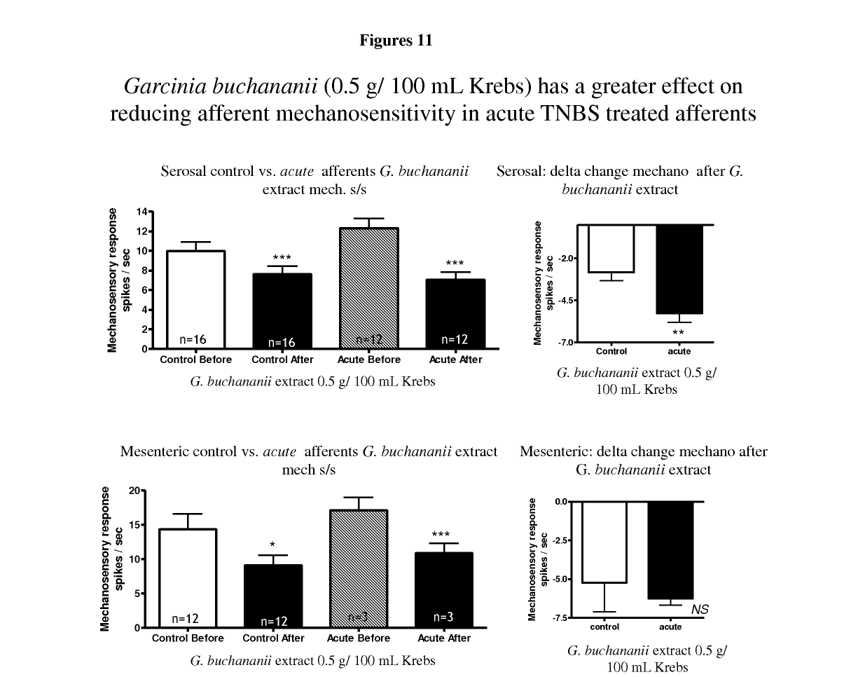

In healthy mice the extract evoked direct chemosensory responses in 81% of serosal and 33% of mesenteric afferents (FIG. 11). Furthermore, the extract significantly inhibited afferent firing in response to von Frey probing, although the extent of inhibition was greater in serosal (P<0.001, n=16) compared with mesenteric afferents (P<0.05, n=12). In TNBS-treated mice similar proportions of afferent responded directly to the extract as in healthy mice, with similar magnitudes of response. However, in TNBS-treated mice the extract had a significantly greater effect on reducing afferent mechanosensitivity (P<0.0001, n=12). In particular the extract induced inhibition of TNBS-treated serosal afferents was over double that observed in healthy serosal afferents (P<0.01, n=12-16).

Discussion:

Extracts from G. buchananii bark significantly reduce high-threshold colonic afferent mechanosensitivity. This inhibition is greater during acute inflammation and reverses acute inflammatory mechanical hypersensitivity. As such these reductions in mechanosensory function are likely to underlie the ability of G. buchananii to relieve abdominal discomfort and pain.

### Example 4

### 5-HT₃ and 5-HT₄ Receptors Contribute to the Anti-Motility Effects of Garcinia buchananii Bark Extract in the Guinea-Pig Distal Colon

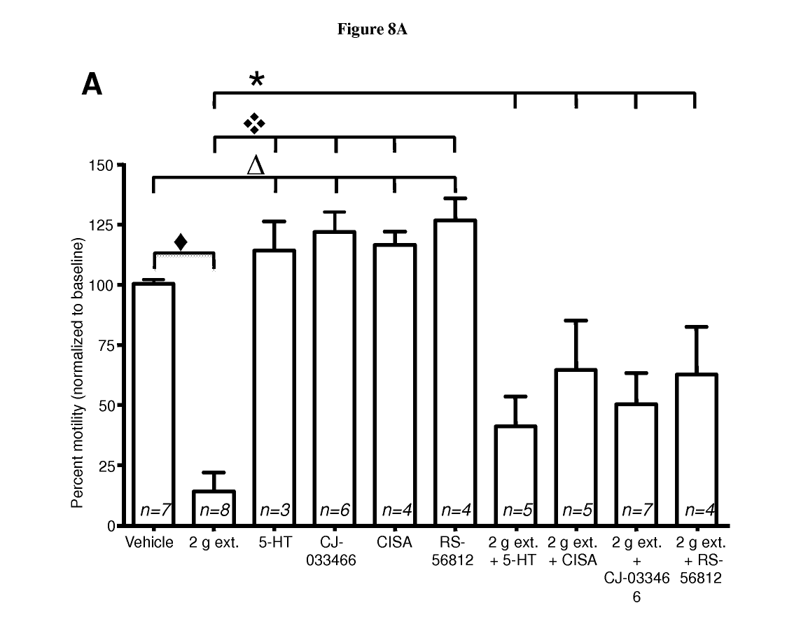

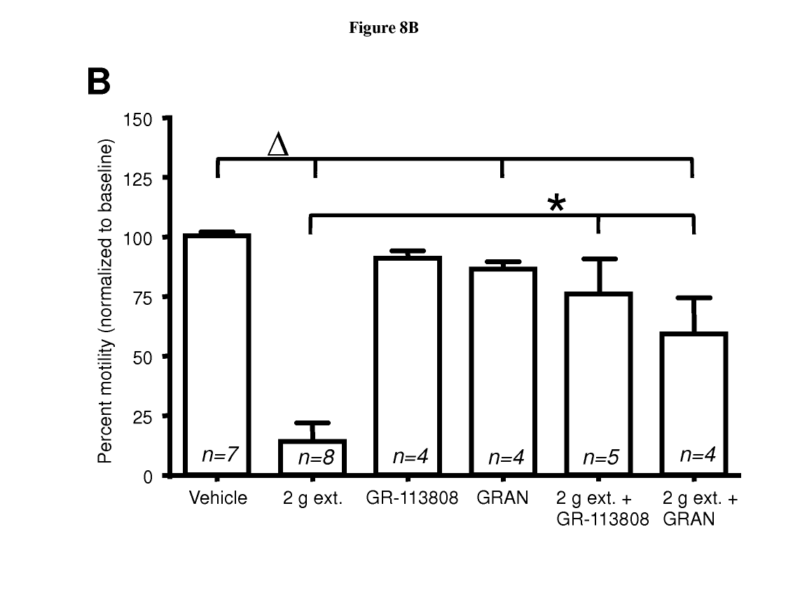

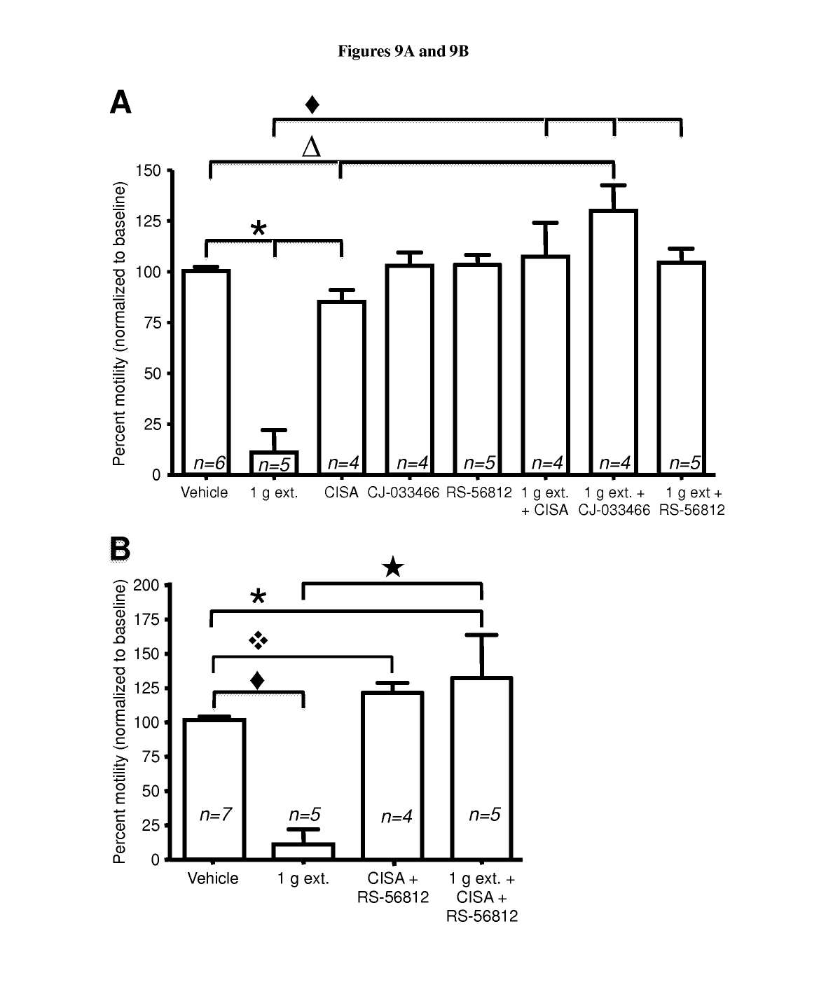

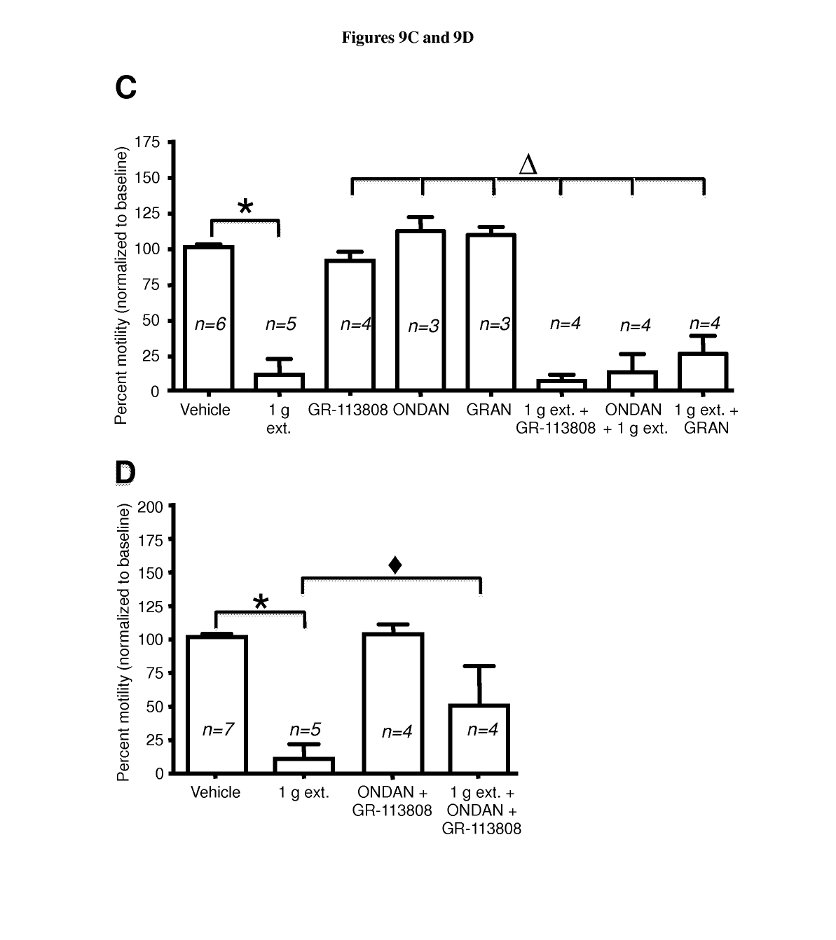

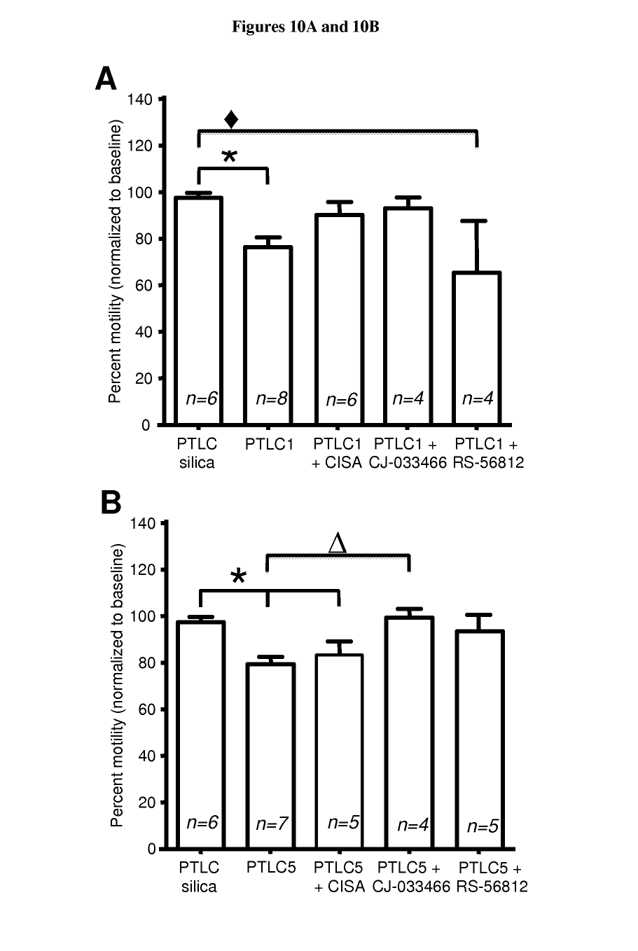

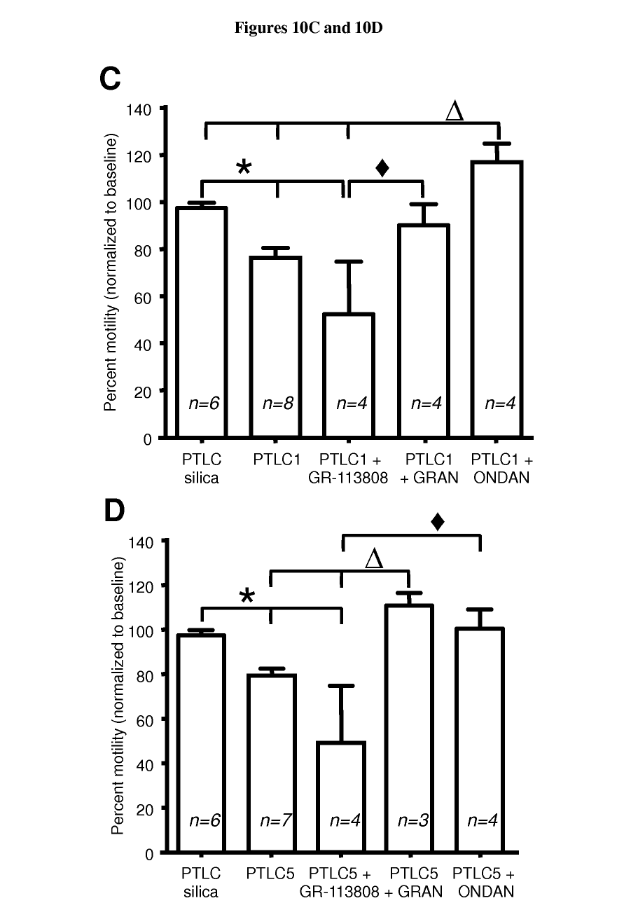

Garcinia buchananii bark extract is an anti-motility diarrhea remedy. It was investigated whether G. buchananii bark extract has components that reduce gastrointestinal peristaltic activity via 5-HT₃ and 5-HT₄ receptors. Methods Aqueous G. buchananii extract was separated into fractions using preparative thin layer chromatography (PTLC) and major chemical components were identified using standard tests. The anti-motility effects of the extract and its fractions (PTLC1-5) were studied through pellet propulsion assays using isolated guinea-pig distal colons. Results Anti-motility (PTLC1 & PTLC5) and pro-motility (PTLC2) fractions were isolated from the extract. Flavonoids and sugars were detected in both fractions, phenols were found only in PTLC1, whereas alkaloids, tannins, and steroids were exclusively found in PTLC5. The potency of the extract applied via the mucosal surface was reduced by 5-HT, 5-HT₃ receptor agonist RS-56812, 5-HT₄ receptor agonists cisapride and CJ-033466 (FIG. 8A), 5-HT₃ receptor antagonist granisetron, and 5-HT₄ receptor antagonist GR-113808 (FIG. 8B). The antimotility effects of the extract applied via the serosal surface was reversed by 5-HT, 5-HT₃ and 5-HT₄ receptor agonists (FIG. 9A-B) but was not affected by the 5-HT₃ and 5-HT₄ receptor antagonists (FIG. 9C). However, mixing ondansetron and GR-113808 with G. buchananii extract (1 g ext.) reduced the anti-motility potency of G. buchananii extract by 40% (FIG. 9D). The anti-motility effects of the aqueous extract and PTLC1&5 when applied serosally were reversed by RS-56812, cisapride and CJ-033466 (FIG. 10 A-B). The 5-HT₃ receptor antagonists, granisetron and ondansetron reduced the effects of the extract to an extent and completely reversed the anti-motility effects of PTLC1&5 (FIG. 10 C-D). GR-113808 inhibited the actions of the extract during the initial 10 minutes, but enhanced the extracts' anti-motility effects after 15 minutes. GR-113808 augmented the anti-motility activities of PTLC1 and 5 by 20% (FIG. 9C; 10C-D). Conclusions and inferences These results indicate that the anti-motility effects of G. buchananii aqueous extract are potentially mediated by compounds that affect 5-HT₃ and 5-HT₄ receptors.

Therefore, the aim of the present study was to determine whether the anti-motility action of G. buchananii extract involves 5-HT dependent mechanisms, operating through 5-HT₃ and 5-HT₄ receptors.

Materials and Methods

Preparation of Garcinia buchananii extract and fractionation using preparative thin layer chromatography (PTLC): G. buchananii stem bark was collected from plants in their natural habitats in Karagwe, Tanzania and processed as described previously by Balemba and colleagues. A sample of bark powder was deposited at the University of Idaho Stillinger herbarium (voucher #159918). An aqueous extract for isolating PTLC fractions was prepared by suspending 10 g G. buchananii bark powder in a 50 mL ethanol/water (70:30) mixture, sonicating for 20 minutes, and filtering using Whatman filter paper no. 4 (Fisher Scientific, Pittsburgh, USA). The filtrate was extracted with 40 mL of hexane to remove the more hydrophobic components from the aqueous extract. The aqueous extract fraction was collected in a round bottom flask and the solvent removed using a rotary evaporator (150 rpm at 50° C.). The sample was then freeze-dried for 24 hours. 10 g bark powder produced 0.9±0.2 g of freeze-dried samples. Each sample was reconstituted using 15 mL 70% ethanol/water mixture to obtain aqueous extract fraction solution (0.06 g/mL freeze-dried sample) for chromatographic separations.

Chromatographic separations were performed using a 20×20 cm, silica gel GF preparative thin layer chromatoghraphy (PTLC) plates (Analtech, Newark, USA). The stationary phase was 1000 μm thick. 8 μL of the aqueous extract fraction solution was applied as 13 mm band to the silica gel plates using a CAMAG Linomat 5 sample applicator equipped with a 100 μL syringe. A total of 72 μl of aqueous extract fraction solution (9 bands) were applied onto each PTLC plate. Separation was accomplished in a glass chromatography chamber using toluene/ethyl acetate/formic acid (30:20:5) as the mobile phase. After development, the PTLC plates were dried on a hot plate (set to low temperature) in a fume hood for 5 min Plates were viewed under a UVLS-28 EL Series UV Lamp (UVP LLC, Upland, Calif., USA) using 365 nm UV light. Five major fractions were identified. Each fraction was scraped into a 50 mL centrifuge tube. Scrapings from 19 PTLC plates were pooled together for each individual fraction. 30 mL of ethanol was added to each fraction. Samples were sonicated for 20 minutes and centrifuged at a speed of 6000 rpm for 5 minutes. The supernatant was emptied into a 50 mL round bottom flask and solvent removed using a rotary evaporator. The weight of each collected fraction was determined Samples were stored at 4° C. Therefore, approximately 1,368 μL (roughly containing 82.08 mg) of purified freeze-dried aqueous extract fraction was applied to the 19 plates used to collect scrapings for isolating PTLC1-5 fractions (see above).

Phytochemical Screening:

Phytochemical screening was carried out using standard chemical methods. Flavonoids were detected by heating 5 g of G. buchananii bark powder in 10 mL ethyl acetate for 3 minutes. The presence of flavonoids was confirmed by a yellow coloration when the filtrate was mixed with 1 mL of 1% ammonia solution. The presence of tannins was confirmed by the appearance of a blue-black coloration after mixing 1% FeCl₃ with 10 mL of 0.5 g G. buchananii powder in distilled water. Steroids were confirmed by observing a reddish-brown ring after mixing 0.5 mL each acetic acid anhydride with 0.5 g G. buchananii powder in methanol, cooling in ice and mixing with both 0.5 mL chloroform and 1 mL of conc. H₂SO₄. Phenols were confirmed by appearance of a bluish-green coloration after mixing 1 mL aqueous extract (5 g G. buchananii bark powder/30 mL water) with 2 mL of FeCl₃ solution. Alkaloids were confirmed by the appearance of an orange color after adding a drop of picric acid to 2 mL aqueous extract (5 g G. buchananii bark powder/30 mL water). These procedures were used to characterize the compound classes found in PTLC1 (15 mg) and PTLC5 (3.8 mg).

Gastrointestinal Motility Assays:

Animals and solutions: All studies were approved by The University of Idaho Animal Care and Use Committee. Male adult guinea-pigs weighing 250-450 g (Elm Hill Breeding Labs, Chelmsford, Mass., USA) housed in metal cages with soft bedding were used in these studies Animals were maintained at 23-24° C. on a 12:12 h light-dark cycle and provided free access to food and water. Midline laparotomy and distal colon collection was performed after isoflurane anesthesia and exsanguinations of individual animals. Colons were stored in ice-chilled Krebs solution (mM: NaCl, 121; KCl, 5.9; CaCl₂, 2.5; MgCl₂, 1.2; NaHCO₃, 25; NaH₂PO₄, 1.2; and glucose 8; all from Sigma, St. Louis, Mo., USA; aerated with 95% O₂/5% CO₂).

A segment (˜10-12 cm long) of distal colon was pinned on either end in a Sylgard-lined 50 mL organ bath being continuously perfused with oxygenated Krebs solution (rate: 10 mL min⁻¹) and maintained at temperatures between 36-37° C. Nail-polish-coated guinea-pig pellets similar in shape and size to the “native” colonic fecal pellets were used to determine propulsive velocities using the Gastrointestinal Motility Monitoring system (Med-Associates Inc., Saint Albans, Vt., USA) for filming pellet propulsion as described by Hoffman and others (2010). Velocity was calculated based on the time required for pellets to traverse 5-6 cm of the central colon segment ending 1 cm from the aboral end. Data collection started after 30 minutes of equilibration. A five-minute interval was used to allow equilibration of motor activity between successive pellet runs. Baseline velocities were measured for 20 minutes.

In intraluminal studies, the aqueous extract was prepared by suspending 2 g of G. buchananii bark powder in 100 mL Krebs, whereas 1 g G. buchananii bark powder prepared in the same manner was used for bath (serosal) applications. The extract-Krebs mixture was continuously stirred for 30 minutes and filtered using analytical filter paper (Schleicher and Schuell Blue Ribbon filter paper 589/3; 0.2 μm retention). Garcinia buchananii extract and test compounds were delivered to isolated colons in Krebs solution, and evaluated for effects on pellet propulsion during 20-25 minute time intervals. Intraluminal drug deliveries (350 μL min⁻¹ Krebs solution) were performed using polyethylene tubing (PE 205; outside diameter 9.5 mm) inserted ˜1.5 cm into the oral end of the segments. In bath deliveries, G. buchananii extract and test compounds were superfused directly into the organ bath. Studies involving PTLC fractions were performed using the organ bath delivery method only. In initial pellet propulsion assays, we found that PTLC1 and PTLC5 obtained by collecting scrapings from 19 PTLC plates produced enough PTLC1 and PTLC5 fractions to significantly reduce pellet propulsion. Therefore, scrapings from PTLC 19 plates were used for each pellet propulsion assay in order to roughly maintain their proportionality relative to the bark powder and the aqueous extract fraction, used for chromatographic separations (see above).

Data Analysis:

To reduce variability between trials and between experiments, pellet propulsion velocity (mm sec⁻¹) for each trial was expressed as a percentage of normalized baseline velocity as described previously by Balemba, O.B. and others, 2010. Normalized control data (Krebs vehicle) passed the Shapiro-Wilk normality test (w=0.93 and P=0.20 for intraluminal applications and w=0.96 and P=0.27 for bath applications). Statistical analysis of all data was performed using one-way ANOVA and the Newman-Keul's multiple comparison post-test and Student's t-test using GraphPad Prism 5 software (GraphPad Software Inc., San Diego, Calif., USA). Data are expressed as Mean±SEM. Differences were considered statistically significant at P<0.05.

Drugs:

Serotonin hydrochloride (5-HT), CJ-033466, GR-113808, RS-56812 hydrochloride and granisetron hydrochloride were purchased from Tocris Bioscience (Ellisville, Mo., USA). Cisapride and ondansetron hydrochloride were purchased from Sigma-Aldrich (Saint Louis, Mo., USA). Stock solutions (1-5 mmol L⁻¹) were prepared by dissolving 5-HT, ondansetron and granisetron in water. Other drugs were dissolved in DMSO. The final dilution of DMSO in Krebs was less than 1:10,000.

Results

Phytochemical Composition of Aqueous G. buchananii Extract and its Anti-Motility Fractions

Using fluorescent color indexes, we identified five distinct regions after PTLC separation of G. buchananii extract. These regions were designated as PTLC1-5 (FIG. 5A). Upon further separation using high performance thin layer chromatography, each PTLC fraction revealed 3-5 sub-fractions (FIG. 5B). A deeper analysis of PTLC1-5 using UPLC-TOF-MS showed that each PTLC fraction contained a minimum of three distinct compounds (FIG. 6). Initial screening for the classes of compounds commonly found in Garcinia species revealed that aqueous G. buchananii extract contains flavonoids, sugars, glycosides, alkaloids, tannins, phenols and steroids (Table 1). These compound classes were also found in PTLC1 and PTLC5, the G. buchananii extract fractions that inhibited pellet motility (Table 2; FIG. 7). While flavonoids and sugars were detected in both fractions, phenols were found only in PTLC1 and alkaloids, tannins and steroids were exclusively found in PTLC5. PTLC 2, 3 and 4 were not subjected to chemical testing, as preliminary data showed them to have minimal anti-motility effects.

Baseline Pellet Velocity and the Actions of Aqueous G. buchananii Extract.

The aqueous G. buchananii bark extract prepared from 2 g bark powder did not alter the pH of Krebs (7.38+/−0.05). The baseline pellet velocity obtained with intraluminal superfusion of Krebs solution was 3.04±0.15 mm sec⁻¹ (n=26), which was similar to that recorded by superfusing Krebs into the organ bath (2.76±0.12; n=34; P=0.15). Intraluminal application of G. buchananii extract (2 g bark powder/100 mL Krebs) had no effect for the first 10 minutes (P>0.05), but inhibited pellet propulsion by 25% (P=0.002) after 15 minutes and 80-90% (P=0.0001) after 20 minutes (Tables 3A-B). Organ bath delivery of G. buchananii extract (1 g bark powder/100 mL Krebs) reduced pellet velocity by 50% after 5 minutes (42.7±16.4% VS. 101.7±2.5%; P<0.05) and 80-95% after 10 minutes (30.2±24.1% VS. 101.7±2.5%; P<0.05).

The Aqueous G. buchananii Bark Extract Contains Anti-Motility and Pro-Motility Components.

Organ bath applications of the PTLC fractions was used to study the effect of PTLC fractions on pellet propulsion in the guinea-pig distal colon in an effort to understand the variety of anti-motility components present in G. buchananii extract. The bath application approach was chosen because previous studies showed that G. buchananii exerts anti-motility effects with greater potency when applied in this manner as a result of inhibiting neurotransmission in the myenteric ganglia. We found that fractions from aqueous G. buchananii extract (PTLC1-5) had variable effects on colon motility. Control silica (PTLC Silica: 1 mg Krebs/100 mL), PTLC3 (10 mg/100 mL Krebs) and PTLC4 (0.9 mg/100 mL Krebs) did not affect pellet propulsion (Table 2; P>0.05; 20 minutes). Somewhat surprisingly, given that the overall effect of G. buchananii extract is to inhibit motility, PTLC2 (28 mg/100 mL Krebs) exhibited pro-motility effects, significantly increasing pellet velocity after 20 minutes (FIG. 7; Table 2; P<0.05). Conversely, PTLC1 (15 mg/100 mL Krebs) and PTLC5 (3.8 mg/100 mL Krebs) decreased pellet propulsion to a significant level (FIG. 7; P<0.05). Compared with PTLC silica, a combination of PTLC1 and PTLC5 inhibited pellet propulsion (P<0.05). The magnitude of this inhibition was similar to that observed with either PTLC1 or PTLC5, when they were applied individually (P>0.05). These results suggest that G. buchananii extract contains a variety of anti-motility and pro-motility components. We chose to focus on the anti-motility effects of G. buchananii extract, thus PTLC fractions having anti-motility effects were subjected to further investigation concerning how they affect 5-HT₃ and 5-HT₄ receptor activities.

5-HT₄ and 5-HT₃ Receptor Agonists and Antagonists Inhibit Anti-Motility Effects Normally Elicited by Mucosal Surface Application of G. buchananii Extract

To determine the roles of 5-HT₃ and 5-HT₄ receptors on G. buchananii extract activity, numerous compounds were applied intraluminally, either alone or in combination with the aqueous G. buchananii extract (2 g extract). These compounds were: the endogenous 5-HT agonist, 5-HT (0.5 μmol L⁻¹); the partial 5-HT₃ receptor agonist, RS-56812 (50 nmol L⁻¹); and the 5-HT₄ receptor agonists, cisapride (100 nmol L⁻¹) and CJ-033466 (300 nmol L⁻¹). All compounds increased pellet propulsion (Table 3A; FIG. 8A; P<0.05). When 5-HT, RS-56812, cisapride and CJ-033466 each were applied in combination with the extract, they reduced the anti-motility potency of G. buchananii extract (Table 3B; FIG. 8A; P<0.05; 20 minutes). In summary, 5-HT, 5-HT₃ and 5-HT₄ receptor agonists reduced the anti-motility actions of the extract elicited from the mucosal side and there was no difference between the effects of these agonists.

In another set of experiments, we found that 5-HT₃ and the 5-HT₄ receptor antagonists granisetron and GR-113808 also reduced the efficacy of G. buchananii extract (Table 3B; FIG. 8B). When applied for 20 minutes in the absence of the extract, granisetron (2 μM) reduced pellet propulsion by 15% (Table 3A; FIG. 8B; P<0.05) while GR-113808 (5 μM) caused a 10% decline (FIG. 8B; P>0.05). Combining granisetron with G. buchananii extract reduced the potency of the extracts' anti-motility effects by 50% (Table 3B; FIG. 8B; P=0.001), whereas GR-113808 reduced the efficacy of G. buchananii extract anti-motility effects by 70% (Table 3B; FIG. 8B; P<0.001; 20 minutes). In summary, the 5-HT₄ antagonist, GR-113808 and the 5HT₃ receptor antagonist, granisetron, inhibited the actions of the extract elicited from the mucosal side.

5-HT₃ and 5-HT₄ Receptor Agonists Reverse the Anti-Motility Effects of G. Buchananii Extract Elicited During Serosal Surface Application

Organ bath application of 5-HT₃ receptor agonist RS-56812 and 5-HT₄ agonist CJ-033466 did not affect pellet velocity (FIG. 9A; 101.7±2.5% vs 103.3±4.7% and 102.8±6.5%; each P>0.05), whereas the 5-HT₄ agonist cisapride reduced it by 15% (101.7±2.5% vs 85.0±5.5%; P<0.05). However, Combining 1 g G. buchananii extract with RS-56812 or cisapride completely reversed the anti-motility effects of the extract 104.3±6.9% and 107.4±16.7% vs 11.0±11.0%; P<0.05), while combining G. buchananii extract with CJ-033466 actually increased pellet velocity beyond that of vehicle (129.8±12.6% vs 101.7±2.5%; P<0.05) and cisapride alone (129.8±12.6% vs 85.0±5.9%; P<0.05). Furthermore, when RS-56812 and cisapride were tested together with the extract, pellet velocity was increased beyond that of vehicle (FIG. 9B; 132.5±31.4% vs 101.7±2.5%; P<0.05). In summary, the 5-HT3 and 5-HT4 receptor agonists reversed the extract's anti-motility actions elicited from serosal side. There was no difference between the effects of the agonists tested.

5-HT₄ and 5-HT₃ Receptor Antagonists Affect the Anti-Motility Action of G. buchananii extract normally elicited during serosal surface application

In organ bath deliveries, 5-HT₃ antagonists granisetron (1 μmol L⁻¹) and ondansetron (0.5 μmol L⁻¹) did not alter pellet propulsion (FIG. 9C; 101.7±2.5% vs 101.4±7.0% and 111.3±9.8%; P>0.05). Contrary to the intraluminal findings, G. buchananii extract reduced pellet propulsion in the presence of ondansetron and granisetron with magnitudes that were similar to that of the extract alone (FIG. 9C; 12.8±12.7% and 25.6±12.5% vs 11.0±11.0%; P>0.05; 20 minutes). In a manner similar to the 5-HT₃ antagonists, 5-HT₄ receptor antagonist GR-113808 (5 μmol L⁻¹) did not did not alter pellet propulsion for up to 20 minutes. Garcinia buchananii extract did not affect pellet propulsion during the initial 5-10 minutes of application in the presence of GR-113808 (101.7±2.5% vs 129.7±22.8%; P>0.05; 5 minutes). However, after 20 min, the velocities had dropped to levels slightly below those elicited by G. buchananii alone (4.2±4.0% vs 11.0±11.0%; P>0.05). Compared with vehicle, combining ondansetron and GR-113808 did not affect pellet velocity (FIG. 9D; 101.7±2.5% vs 104±7.3%; P>0.05). Although the effect was not significant, mixing GR-113808 and ondansetron with G. buchananii extract reduced the efficacy of the extract's anti-motility effects by 40% (FIG. 9D; 50.5±29.5% VS. 11.0±11.0%; P<0.01). In summary, the 5-HT₃ receptor antagonists did not significantly affect the actions of G. buchananii extract. 5-HT₄ receptor antagonist GR-113808 inhibited the extract for 5-10 minutes. Combining 5-HT₃ and 5-HT₄ antagonists weakened the efficacy of the extract to a much greater extent.

5-HT₃ and 5-HT₄ Receptor Agonists Reverse the Anti-Motility Effects Normally Elicited from Serosal Surface by PTLC1 and PTLC5 Fractions

Based on the observations that G. buchananii extract failed to inhibit pellet propulsion in the presence of 5-HT₃ receptor agonist RS-56812 and 5-HT₄ receptor agonists cisapride and CJ-033466, we investigated whether RS-56812, cisapride, and CJ-033466 affected the anti-motility effects of PTLC1 (15 mg) and PTLC5 (3.8 mg). Compared with PTLC silica, application of PTLC1 and PTLC5 for 20 min did not reduce pellet propulsion in the presence of CJ-033466 (FIG. 10A-B; 97.5±2.2% vs 99.4±4.5%; P>0.05), which is similar to G. buchananii extract. Cisapride inhibited the anti-motility effects of PTLC1 (FIG. 10A; 97.5±2.2% vs 90.3±5.4; P>0.05), but failed to inhibit anti-motility effects of PTLC5 (FIG. 10B; 97.5±2.2% vs 83.2±5.9%; P<0.05). RS-56812 failed to inhibit anti-motility effects of PTLC1 (FIG. 10A; 97.5±2.2% vs 65.3±22.3%; P<0.05), but completely reversed the effects of PTLC5 (FIG. 10B; 97.5±2.2% vs 93.8±6.5%; P>0.05). In summary, CJ-033466 inhibited the effects of PTLC1 and PTLC5, whereas cisapride inhibited PTLC1, but not PTLC5. The 5HT₃ receptor agonist, RS-56812, inhibited the effect of PTLC5, but not PTLC1.

5-HT₃ and 5-HT₄ Receptor Antagonists Affect PTLC1 and PTLC5 Anti-Motility Effects Elicited from Serosal Surface

When compared with PLTC silica, serosal application (20 min) of PTLC1 in the presence of ondansetron (0.5 μmol L⁻¹) and granisetron (1 μmol L⁻¹) did not affect pellet propulsion (FIG. 10C; 97.5±2.2% vs 116.9±7.9% and 90.2±8.8%; P>0.05). The combination combination of ondansetron and PTLC1 actually increased pellet propulsion (FIG. 5C; 97.5±2.2% vs 116.9±7.9%; P<0.05). In contrast, PTLC1 reduced pellet velocity with greater potency in the presence of 5-HT₄ receptor antagonist GR-113808 (FIG. 10C; 97.5±2.2% vs 52.4±22.4%; P<0.001). Like PTLC1, PTLC5 did not affect pellet propulsion in the presence of ondansetron and granisetron (FIG. 10D; 100.4±8.5 and 110.7±5.7% vs 97.5±2.2%; P>0.05). In addition, PTLC5 inhibited pellet propulsion with greater efficacy in the presence of GR-113808 (97.5±2.2% vs 49.1±35.6%; P<0.001). In summary, the 5-HT₄ receptor antagonist augmented the actions of PTLC1 and PTLC5, whereas the 5HT₃ receptor antagonists entirely inhibited the anti-motility actions of both fractions.

Discussion

The goal of this study was to determine whether the anti-motility effect of G. buchananii extract involves 5-HT₃ and 5-HT₄ receptors, and whether the extract can be separated into fractions that have similar effects. The extract was initially separated into five fractions; two with anti-motility effect, one with pro-motility effect, and two without any effect. This suggests that G. buchananii may be a valuable source of novel anti-motility and pro-motility compounds, or their derivatives. The ability of G. buchananii extract and its fractions to inhibit guinea-pig colonic motility was reversed by 5-HT₃ and 5-HT₄ receptor agonists, and variably affected by antagonists of these receptors. This indicates that the anti-motility bioactive components in the extract affect 5-HT₃ and 5-HT₄ receptors signaling either directly or indirectly. G. buchananii aqueous extract had a greater effect on 5-HT₄ receptors, especially during mucosal application, while the isolated fractions showed higher efficacy with 5-HT₃ receptors compared with 5-HT₄ receptors. Overall, our findings suggest that G. buchananii extract contains several anti-motility compounds that exert different effects on 5-HT₃ and 5-HT₄ receptors through mechanisms that remain to be determined

Garcinia buchananii is a Potential Source of Anti-Motility and Pro-Motility Compounds

There is a great deal of clinical and scientific evidence regarding major breakthroughs in treating diarrheal diseases using ORT, synthetic drugs especially opiate- and enkephalin-based formulations and vaccinations. However, current therapies do not necessarily shorten the duration of diarrhea, or lessen abdominal pain, and most synthetic drugs are limited by side effects (mainly constipation, dependency, and safety for children and pregnant women). The alteration in enteric neurotransmission is a major contributor to increased enteric secretion, hypermotility and cramping pain observed in diarrheal diseases. Therefore, there is the critical need for novel anti-diarrheal agents that target enteric neurotransmission. Botanical products and their derivatives have historically led to the discoveries of effective modern drugs and have the potential to fulfill this requirement. G. buchananii bark extract is a potential source of new anti-diarrhea drugs. This notion is supported by our previous findings that G. buchananii bark extract is a non-opiate preparation that reduces propulsive motility by inhibiting synaptic transmission in the myenteric ganglia. Additional support comes from the observations of this study that the extract's anti-motility effect may involve inhibition of 5-HT₃- and 5-HT₄-receptors.

Individual extract fractions (PTLC1 and PTLC5) with anti-motility effects produced less than 25% reduction of propulsive motility and their effects were not additive, while the extract alone inhibited motility by more than 70%. The reasons for these observations are unclear at this time. It is possible that the extract contains larger levels of bioactive compounds than PTLC1 and PTLC5 since increasing the amount of PTLC1 or PTLC5 caused greater inhibition of motility (Boakye and Balemba personal observations). We cannot rule-out, however, that the separation may have altered biological actions. Alternatively, despite the overall anti-motility effect of PTLC 1 and 5, these fractions contain sub-fractions (see FIGS. 5B; 6), which may have pro-motility properties that can reduce the degree of motility inhibition originally evoked by PTLC1 and PTLC5.

5-HT₃ and 5-HT₄ Receptors Play a Crucial Role in the Anti-Motility Effect of Bioactive Components of G. buchananii Extract

It is evident that 5-HT₃ and 5-HT₄ agonists reversed the effects of the extract and its fractions. Similarly, the 5-HT₃ and 5-HT₄ receptor antagonists either inhibited the anti-motility actions of G. buchananii extract and its fractions or to a degree augmented the effects of the fractions. These initial studies show that the crude extract and fractions contain multiple bioactive components and this suggests complicated effects on the effector tissues of colon motility. Overall, our study indicated that the bioactive compound(s) have a serotonergic effect, and provides the basis of future experiments using purified compounds. Our findings were similar to observations that wood creosote, a botanical preparation that stopped stress-induced diarrhea by inhibiting 5-HT₃ and 5-HT₄ receptors in rat colon. The observation of remarkable extract's anti-motility after 20 minutes supports the notion that concurrent inhibition of these receptors drastically reduces colonic motility. Agents that inhibit 5-HT₄ receptors or both 5-HT₃ and 5-HT₄ receptors reduce both the ascending and descending peristaltic reflexes. Furthermore, anti-emetic drugs act by inhibiting 5-HT₃ and 5-HT₄ receptors, and 5-HT₃ receptor antagonists are used as medications for functional bowel disorders and diarrhea-predominant IBS. Therefore, G. buchananii bark extract could also be a source of new compounds for the treatment of nausea and vomiting, functional bowel disorders and diarrhea-predominant IBS Also, the extract may be beneficial in pain management associated with these conditions. It has been shown that steroids have anti-inflammatory, anti-motility and anti-secretory effects in patients with collagenous colitis. Steroids have been shown to contribute to the anti-inflammatory activity of Garcinia extracts. Whether the steroids found in G. buchananii extract and PTLC5 have anti-inflammatory actions and also reduce motility is currently unclear.

The effect of the bioactive components of G. buchananii extract on 5-HT₃- and 5-HT₄-receptor signaling in the guinea-pig distal colon appears unique. The reasons for this claim is that the effects of G. buchananii extract and its fractions were inhibited by 5-HT₃ and 5-HT₄ receptor agonists and antagonists, or to some extent augmented by 5-HT₄ antagonist or a combination of 5-HT₃ and 5-HT₄ antagonists. The findings using PTLC1 and PTLC5 suggest differences in the relative contribution of 5-HT₃ and 5-HT₄ receptors to the anti-motility effect of the fractions with the 5-HT₃ receptors being the major effector. In contrast, 5-HT₄ receptors appeared to be the primary receptors affected by crude G. buchananii extract. It is possible that PTLC1 and PTLC5 fractions contain different bioactive compounds, or that PTLC fractionation altered biological activity of the active compounds, thus changing how they affect 5-HT receptors. Regardless of these differences, our results bolster the idea that the efficacy of bioactive compounds in G. buchananii extract involved in reducing colon motility depends on an inhibition of 5-HT₃ and 5-HT₄ receptors.

Gamma-mangostin, a xanthone from Garcinia cambogia fruit extracts interacts with 5-HT_{2A} receptors. Here, it was demonstrated for the first time that G. buchananii extract contains compounds that inhibit intestinal motility via 5-HT₃- and 5-HT₄-receptors Botanical compounds that have been shown to inhibit 5-HT₃- and 5-HT₄-receptors are flavonoids, phenolics, creosol, guaiacol and 4-ethylguaiacol. The compounds in G. buchananii extract (Table 1) that actively interact with the receptors therefore appear to be flavonoids and/or phenols. Whether steroids, alkaloids and tannins are involved should also be considered.

We have previously proposed that the rebound of pellet propulsion during washout following treatment of isolated guinea-pig colon with G. buchananii extract is due to pro-motility components in the extract. This study provides evidence to support the existence of pro-motility compounds in G. buchananii bark extract. It is possible that GABA receptors have a role in the pro-motility effect of PTLC2.

Intraluminal and serosal deliveries of 5-HT₃ and 5-HT₄ agonists show several differences. The agonists augmented motility while antagonists inhibited propulsion during intraluminal perfusion. In contrast, (with the exception of cisapride), agonists and antagonists did not affect pellet velocity when applied to the serosal side. Garcinia buchananii bark extract inhibits pellet propulsion rapidly from the serosal side. It is believed that this effect is due to the inhibition of synaptic neurotransmission in the myenteric ganglia presumably by inhibiting pre-synaptic release of acetylcholine, or by other mechanisms and neurotransmitters. While the findings of this study suggest serotonergic effects, a clear interpretation of the 5-HT₃ and 5HT₄ agonists ability to reverse extract inhibition will require further studies using purified compounds.

Conclusions:

Garcinia buchananii bark extract contains bioactive compounds that interact with 5-HT₃ and 5-HT₄ receptors to inhibit peristaltic activity.

Tables

TABLE 1
Phytochemical analysis of G. buchananii extract and anti-motility
fractions PTLC1 and PTLC5.

| | G. buchananii Aqueous | | |
|---|---|---|---|
| Compound | extract | PTLC1 | PTLC5 |
| Flavonoids | ++++ | +++ | ++ |
| Glycosides | +++ | N/A | N/A |
| Sugars | ++++ | +++ | + |
| Alkaloids | +++ | − | +++ |
| Tannins | +++ | − | +++ |
| Phenols | +++ | N/A | N/A |
| Steroids | ++++ | − | +++ |
| Anthraquinones | − | ND | ND |
| Phlobatannins | − | ND | ND |
| Terpenoids | − | ND | ND |
| Saponins | − | ND | ND |

Key:
Qualitative assessment of the classes of natural chemical compounds found in G. buchananii bark extract and PTLC fractions having anti-motility effects.
‘+’ indicates the compound was present and
‘−’ depicts the compound was absent.
‘N/A’ refers to an inability to perform the desired chemical test.
‘ND’ refers to not done since the component was not found in the extract.
Reaction intensity scores: + = low intensity; ++ = medium intensity, +++ = high intensity and ++++ = very high intensity.

TABLE 2
Bath application: The effect of 1 g G. buchananii extract and PTLC1-PTLC5 fractions on pellet
propulsion (expressed as a percent of velocity due to treatment, divided by normalized baseline
pellet velocity)

| | | | PTLC silica | PTLC1 (15 mg/ | PTLC2 |
|---|---|---|---|---|---|
| Duration | Vehicle | 1 g extract | 1 mg/100 mL; | 100 mL); | (28 mg/100 mL) |
| (min.) | (n = 6) | (n = 5) | (n = 6) | (n = 8) | (n = 7) |
| 5 | 102.1 ± 2.1% | 42.7 ± 16.4% | 106.3 ± 4.7% | 89.3 ± 4.5% | 98.3 ± 4.7% |
| | | *P < 0.0001 | P > 0.05 | *P > 0.05 | P > 0.05 |
| 10 | 100.3 ± 1.9% | 39.2 ± 24.1% | 101.4 ± 2.5% | 78.9 ± 4.8% | 106.7 ± 5.2% |
| | | *P < 0.0001 | P > 0.05 | *P < 0.05 | P > 0.05 |
| 20 | 102.5 ± 2.5% | 11.0 ± 11.0% | 97.5 ± 2.2% | 76.4 ± 4.2% | 114.3 ± 7.2% |
| | | *P < 0.0001 | P > 0.05 | *P < 0.05 | *P < 0.05 |

| | PTLC3 | PTLC4 (0.9 mg/ | PTLC5 (3.8 mg/ | PTLC1 + |
|---|---|---|---|---|
| Duration | (10 mg/100 mL) | 100 mL) | 100 mL | PTLC5; |
| (min.) | (n = 7) | (n = 5) | Krebs; (n = 7) | (n = 4) |
| 5 | 88.5 ± 8.4% | 92.6 ± 6.7% | 91.7 ± 6.4% | 99.6 ± 3.6% |
| | P > 0.05 | P > 0.05 | P > 0.05 | P > 0.05 |
| 10 | 106.3 ± 2.4% | 95.5 ± 4.1% | 82.5 ± 2.7% * | 92.8 ± 3.7% |
| | P > 0.05 | P < 0.05 | P > 0.05 | P > 0.05 |
| 20 | 100.5 ± 8.1% | 114.3 ± 4.7% | 79.3 ± 3.2% | 74.1 ± 9.0% |
| | P > 0.05 | P > 0.05 | * P < 0.05 | *P < 0.05 |

Key:
P values were obtained by using One-way ANOVA and the Newman-Keul's multiple comparison post-test to compare G. buchananii extract and PTLC silica with vehicle. PTLC fractions 1-5 were compared with PTLC silica. Asterisks indicate a significant change.

TABLE 3A
Intraluminal delivery: Control experiments of G. buchananii extract, 5-HT₃ and 5-HT₄ receptor agonists and antagonists.

| Duration | Vehicle (n = | 2 g extract | | | CJ-033466 | GR 113808 | | Granisetron |
|---|---|---|---|---|---|---|---|---|
| (min) | 7) | (n = 8) | 5-HT (n = 3) | Cisapride (n = 4) | (n = 6) | (n = 4) | RS 56812 (n = 4) | (n = 4) |
| 5 | 101.9 ± 2.9% | 97.0 ± 4.3% | 118.3 ± 16.2% | 115.6 ± 4.9% | 132.7 ± 7.1% | 109.3 ± 6.1% | 119.7 ± 5.2% | 99.3 ± 4.2% |
| | | P > 0.05 | *P < 0.05 | *P < 0.05 | *P < 0.05 | P > 0.05 | P > 0.05 | P > 0.05 |
| 15 | 101.2 ± 1.7% | 76.2 ± 11.1% | 117.6 ± 13.5% | 109.6 ± 9.3% | 112.1 ± 6.7% | 93.2 ± 3.6% | 119.6 ± 5.4% | 92.4 ± 1.4% |
| | | *P < 0.05 | P > 0.05 | P > 0.05 | P > 0.05 | P > 0.05 | P > 0.05 | P > 0.05 |
| 20 | 100.6 ± 1.6% | 7.8 ± 6.0% | 114.3 ± 11.9% | 122.0 ± 8.2% | 116.6 ± 5.6% | 91.7 ± 3.1% | 126.7 ± 9.2% | 86.5 ± 3.6% |
| | | *P < 0.0001 | *P < 0.05 | *P < 0.05 | *P < 0.05 | P > 0.05 | *P < 0.0001 | *P < 0.05 |

Key:
P values were obtained using One-way ANOVA and the Newman-Keul's multiple comparison post-test to compare treatments with vehicle. Asterisks indicate a significant change.

TABLE 3B
Intraluminal delivery of G. buchananii extract in combination with 5-HT₃ and 5-HT₄ agonists and antagonists

| | | | 2 g extract + | 2 g extract + | | 2 g | | |
|---|---|---|---|---|---|---|---|---|
| Duration | | 2 g extract | 5-HT | cisapride | 2 g extract + CJ- | extract + GR- | 2 g extract + RS- | 2 g extract + |
| (min) | Krebs (n = 7) | (n = 8) | (n = 5) | (n = 5) | 033466 (n = 7) | 113808 (n = 4) | 56812 (n = 4) | GRAN (n = 4) |
| 5 | 101.9 ± 2.9% | 97.0 ± 4.3% | 104.3 ± 16.1% | 99.2 ± 4.9% | 97.9 ± 8.0% | 101.6 ± 5.8% | 115.5 ± 13.2% | 86.3 ± 4.8% |
| | | | P > 0.05 | P > 0.05 | P > 0.05 | P > 0.05 | P > 0.05 | P > 0.05 |
| 15 | 101.2 ± 1.7% | 76.2 ± 11.1% | 111.0 ± 4.7% | 98.4 ± 19.4% | 66.7 ± 14.2% | 89.7 ± 14.9% | 89.3 ± 20.6% | 66.7 ± 16.4% |
| | | | P > 0.05 | P > 0.05 | P > 0.05 | P > 0.05 | P > 0.05 | P > 0.05 |
| 20 | 100.6 ± 1.6% | 7.8 ± 6.0% | 41.1 ± 12.4% | 66.5 ± 20.6% | 50.2 ± 13.1% | 76.2 ± 15.7% | 62.7 ± 19.9% | 59.2 ± 15.1% |
| | | | *P < 0.05 | *P = 0.05 | *P < 0.05 | *P < 0.05 | *P < 0.05 | *P < 0.05 |

Key:
P values are based on One-way ANOVA and the Newman-Keul's multiple comparison post-test to compare data of G. buchananii extract + 5-HT₃ and 5-HT₄ agonists as well as G. buchananii extract + 5-HT₃ and 5-HT₄ antagonists with G. buchananii extract alone. Asterisks indicate a significant change. GRAN = granisetron.

### Example 5

### Garcinia buchananii Bark Extract is an Effective Anti-Diarrheal Remedy for Lactose Induced Diarrhea

Ingesting large quantities of lactose (45-87%) causes diarrhea in humans and in animals. Lactose induces severe and persistent diarrhea, malnutrition, and intestinal damage that are similar to what is seen in children suffering from gastroenteritis or chronic diarrhea. The aims of this study were to investigate the effectiveness of G. buchananii bark extract in stopping lactose-induced diarrhea in rats, determine the effective dose, and test the effectiveness of anti-motility fractions isolated using preparative thin layer chromatography as anti-diarrheal agents.

Introduction

Methods:

A high-lactose (35%) diet induced diarrhea in Wistar rats, which were then treated with either G. buchananii bark extract (0.1, 0.5, 1.0 and 5.0 g bark powder) or its anti-motility fractions isolated using preparative thin layer chromatography; termed PTLC1 (15 mg) and PTLC5 (3.8 mg) or loperamide (8.4 mg), each dissolved in 1 liter of tap water. Numerous parameters were measured in each condition including consistency, fluid and mucus content of feces, body weight, water and food consumption, urine production and bloating.

Results:

Diarrheic rats produced watery or loose, mucuoid, sticky, feces. Fluids constituted 86% of stool mass compared with 42% for control rats fed standard chow. Compared with controls, diarrheic rats produced more urine, lost weight and had bloated ceca and colons. All doses of the extract (0.1, 0.5, 1.0 and 5.0 g), the anti-motility fractions and loperamide individually stopped diarrhea within 6-24 hrs of administration, whilst significantly reducing mucus and fecal fluid content, urine production and intestinal bloating. Interestingly, only rats treated with 0.1 g extract and the anti-motility fractions gained weight, whilst PTLC5 also increased water intake.

Conclusions:

Garcinia buchananii is an effective anti-diarrheal remedy. The extract contains compounds that reverse weight loss, promote food and water intake, supporting the notion that characterization of the compounds could lead to new therapies against diarrheal diseases.

Materials and Methods

Inducing Diarrhea in Rats Using a High-Lactose Diet

The study was conducted in accordance with the regulations of the University of Idaho Institutional Animal Care and Use Committee (IACUC). Sixty two, 10 week old Wistar rats were obtained from Harlan Animal Research Laboratory (Hayward Calif., USA). Rats were individually caged (23-24° C.; 12:12 h light-dark cycle) and quarantined for one week. Rats were fed a standard chow diet ad libitum (Animal Specialties, Hubbard, Oreg., USA) and had free access to water for four days prior to the inducement of diarrhea. A high-lactose diet (HLD) containing 35% lactose in place of starch (3.004 Kcal; Purina Mills; Richmond, Ind., USA) was fed to 52 rats. Diarrhea was induced within 24-48 hours after consuming the diet. Rats were monitored for changes in consistency of pellets and stool mass, fecal fluid and urine production (mass; g and volume; ml). Rats were considered diarrheic if they produced watery stools, soft, yellowish stools compared to normal, pliable, soft, well-formed pellets as previously described by other researchers.

Four days after introducing rats to a HLD, diarrheic rats were treated using varying doses of G. buchananii extract, its anti-motility fractions isolated using preparative thin layer chromatoghraphy (PTLC1 & PTLC5) and loperamide for standard comparison. Rats were maintained on a HLD during the entire treatment period.

Preparation of G. buchananii Bark Extract, PTLC1 & PTLC5 Fractions

Garcinia buchananii bark powder was prepared from stem barks collected from trees in their natural habitat in Karagwe, Tanzania, as described previously by Balemba, OB and colleagues, 2010. A sample can be found at the University of Idaho Stillinger herbarium (voucher #159918). 0.1 g, 0.5 g, 1.0 g and 5.0 g of G. buchananii bark powder were each suspended in 1 L of tap water, stirred for 30 minutes and filtered. The filtrate was then immediately used to treat rats against lactose-induced diarrhea.

The anti-motility fractions were obtained from the aqueous G. buchananii bark extract using a preparative thin layer chromatography (PTLC) separation method as described previously.

Treating Diarrheic Rats with G. buchananii Extract, PTLC1 and PTLC5 Fractions

Twenty nine rats on a HLD were randomly assigned and treated with varying doses of G. buchananii extract (0.1 g, 0.5 g, 1 g, and 5 g extract; n=6-10 rats per group). Eight rats were treated with PTLC1 or PTLC5 (each n=4) at a dose of 15 mg and 3.8 mg in 100 mL tap water, respectively. For control treatments, seven rats were treated with loperamide (8.4 mg/L tap water), eight rats were left untreated (HLD control) and ten rats received control standard chow diets (SD). Rats were treated for a total of four days, receiving freshly made drugs every two days.

Monitoring the Effect of Treatments

We studied the impact of a HLD and all the treatments on the consistency, form and appearance of feces. Body weight, food and water intake were measured every two days for the entire period. Diarrheic rats treated with G. buchananii (0.1 g/l, 1.0 g/l) and loperamide were used to study fecal fluid content, stool mass and urine production compared with untreated rats on a HLD and SD control diets (n=3-5). To achieve this, pellets/stools and urine were allowed to drop on non-leaking, polyester plastic mats spread in metal trays placed beneath wired cages housing rats. Pellets/stools and urine were collected every three hours, for 24 hours, for a 12-day period. Disposable polyethylene transfer pipettes (5.5 mL) were used to collect urine. Pellets/stools were collected by using custom-made pieces of Inkjet transparencies. The samples were stored in disposable, polypropylene containers (100 mL) tightly capped to avoid loss of moisture due to vaporization. Polyester mats, polypropylene containers and transfer pipettes were obtained from VWR international LLC, WA, USA.

Cumulative fresh weights of pellets/stools, and urine mass and volume were recorded every 6 hours for 24 hours. At the end of every 24-hour period, pellets and stools were transferred into Pyrex glass beakers and heated in the oven (70° C.; 24-hr) to obtain constant dry weight. Fecal fluid content was obtained by subtracting dry weight from fresh weight. To eliminate variations between animals, especially during diarrhea period, fecal fluid content was expressed as a fraction of fluid weight divided by total fecal fresh weight.

On the last day, all animals were euthanized by isoflurane inhalation and exsanguinated. Gross anatomical evaluation of the size and color of the small intestine, cecum, and colon as well as of the spleen, liver and kidney were recorded. Length and width of cecum were estimated using cotton threads and a ruler.

Results

Effect of a HLD on the Form of Feces, Fecal Fluid Content and Urine Production

We found that all rats fed a SD diet produced lots of well-formed, rounded, oblong fecal pellets (20+/−2 pellets; 5.82+/−0.15 g per day), whilst mucus was not readily observable by visual inspection. However, the consumption of a HLD caused diarrhea in 85% of rats within 24 hours and all rats within 48 hours after the initiation of the HLD. The majority (46%) of diarrheic rats had severe diarrhea characterized by profuse, watery stools visibly containing lots of mucus (Table 4).

TABLE 4
Comparison of the effectiveness of varying doses of G. buchananii
extract, PTLC1, PTLC5 and loperamide on treating high lactose diet-
induced diarrhea in rats.

| | Form of stool after 24 hrs to the 4^{th} day of treatment | | | | |
|---|---|---|---|---|---|
| | | Loose | | | |
| | Watery | stools + | Loose | | |
| | diarrhea + | mucous | stool with | | |
| | mucous | (non- | structural | Soft | Hardened |
| | (incontinence | structural | form of | formed | normal |
| Treatment | or profuse) | stool) | pellet | pellets | pellets |
| Standard Chow diet | − | − | − | ++ | ++++ |
| 35% lactose diet (HLD) | +++ | ++ | + | − | − |
| HLD + 0.1 g/L extract | − | − | ++ | +++ | + |
| HLD + 0.5 g/L extract | − | − | − | +++ | ++ |
| HLD + 1 g/L extract | − | − | − | +++ | +++ |
| HLD + 5 g/L extract | − | − | − | + | ++++ |
| HLD + PTLC1 | − | − | − | +++ | +++ |
| HLD + PTLC5 | − | − | + | +++ | ++ |
| HLD + 8.4 mg/L LP | − | − | − | − | ++++++ |

Key:
LP = Loperamide.
Qualitative assessment score or classification: ‘−’ condition not observed, + = observed once/day; ++ = observed twice/day, +++ = observed 3-4 times/day, ++++ = abundantly observed and ++++++ = almost exclusively observed.

23% of the rats produced wet, mucoid, loose stools. Approximately 12% of rats produced lots of loose, wet, sticky, yellowish stools with or without structural form of pellet (Table 4), which over time, gradually became watery stool. Visual correlation of feeding with diarrhea showed an association between severe diarrhea with ingesting larger amounts of the HLD.

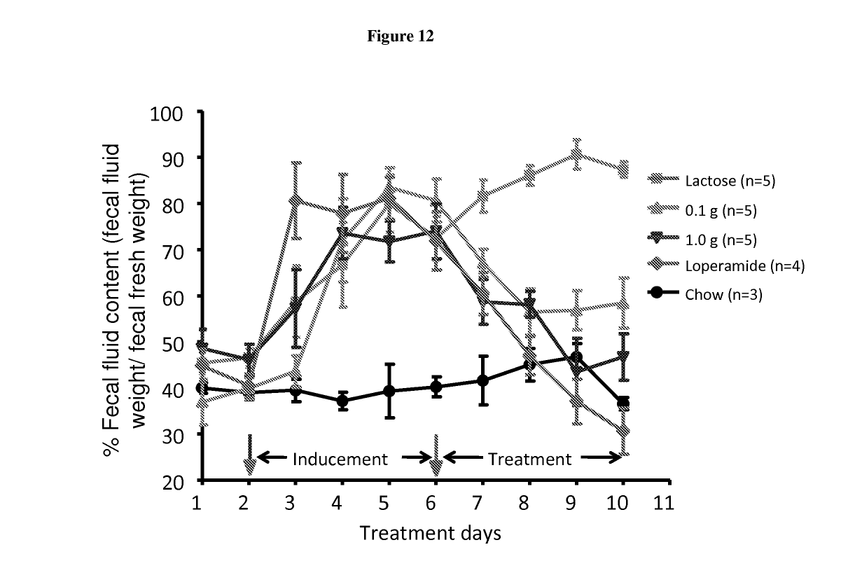

The HLD caused a significant increase in fecal fluid content as fluid content constituted 86% of stool mass compared with 42% in pellets produced by rats fed the SD (FIG. 12; HLD: 86.4±1.9% vs. SO: 42.0±2.3%; P<0.001). Furthermore, the HLD significantly increased urine production compared with SD rats (HLD: 9.32±1.09 g/day vs. SD: 4.68±0.19 g/day; P<0.001). In summary, a HLD successfully induced diarrhea in rats and caused a significant loss of body fluid through diarrhea.

Garcinia buchananii Bark Extract Stopped Diarrhea and Fluid Loss.

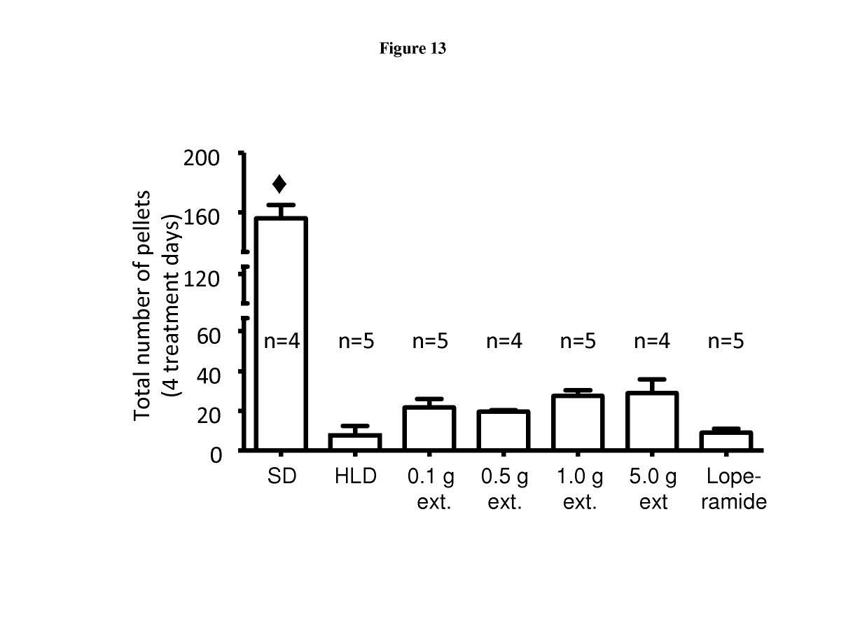

It was found that all doses of aqueous G. buchananii extract were effective in stopping the HLD induced-diarrhea in less than 6 hours after commencing treatment. The form and hardness of feces correlated with the dose and amount of extract consumed. Rats treated with G. buchananii extract (0.1 g/L) produced soft and poorly formed pellets (Table 4; FIGS. 12-13), whilst pellets of rats treated with G. buchananii extract (1.0 g/L) were well-formed, soft, and pliable. Rats treated with G. buchananii extract (5.0 g/L) produced well-formed, rounded, oblong, and relatively harder pellets.

Grossly, the hardness of these pellets was comparable to that of rats treated with loperamide (8.4 mg/L; Table 4). Compared with rats on a HLD alone, G. buchananii extract (0.1 g/L and 1.0 g/L) reduced fecal fluid content in rats with HLD-induced diarrhea (FIG. 12; HLD: 86.4±1.9% vs. 0.1 g extract: 59.7±2.5% and 1.0 g extract: 51.8±3.9%; P<0.01 and P<0.001, respectively), with effects observed as early as 6-24 hours after commencement of treatments. The effect of G. buchananii extract (GB; 1.0 g/L) was similar to that of loperamide (LP; 8.4 mg/L; GB: 51.8±3.9% vs. LP: 43.8±6.5%; P>0.05). In addition, G. buchananii extract (GB; 1.0 g/L) and loperamide reduced the HLD induced increase in urine production (HLD: 16.5+1-1.7 mL vs. CH: 8.4+1-0.5 mL; P<0.01) back to normal SD levels (GB: 8.2+1-0.8 mL and LP: 5.4+1-1.1 mL vs. CH: 8.4+1-0.5 mL; P>0.05, respectively). In summary, the varying doses of aqueous G. buchananii extract were all effective at stopping lactose-induced diarrhea, intestinal mucus secretion, and loss of body fluid through stool and urine. Based on the texture of feces, fluid content, stool fresh weight, and urine production, G. buchananii extract (1.0 g/L) was very effective at treating diarrhea and also promoting fluid retention. Overall, the effects of the extract were comparable to those of loperamide (8.4 mg/L).

A Lower Dose of G. buchananii Bark Extract Also Reversed Weight Loss Due to Lactose-Diet Induced Diarrhea

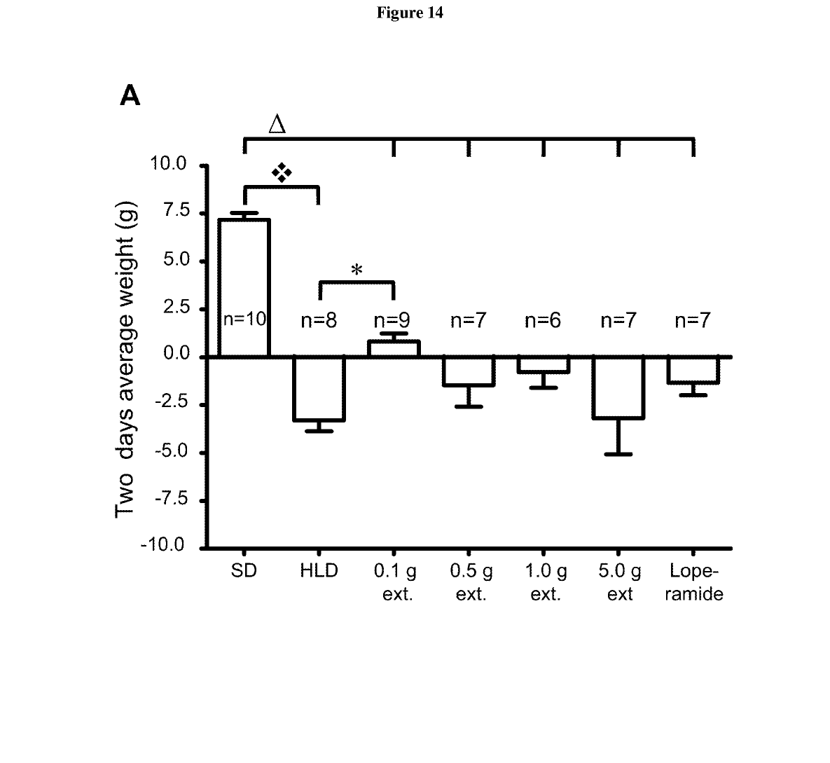

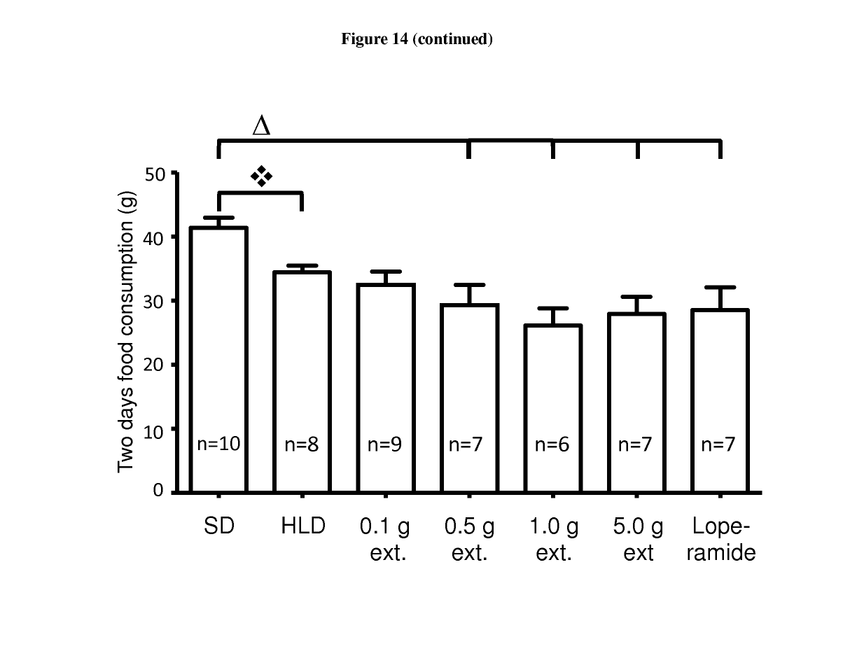

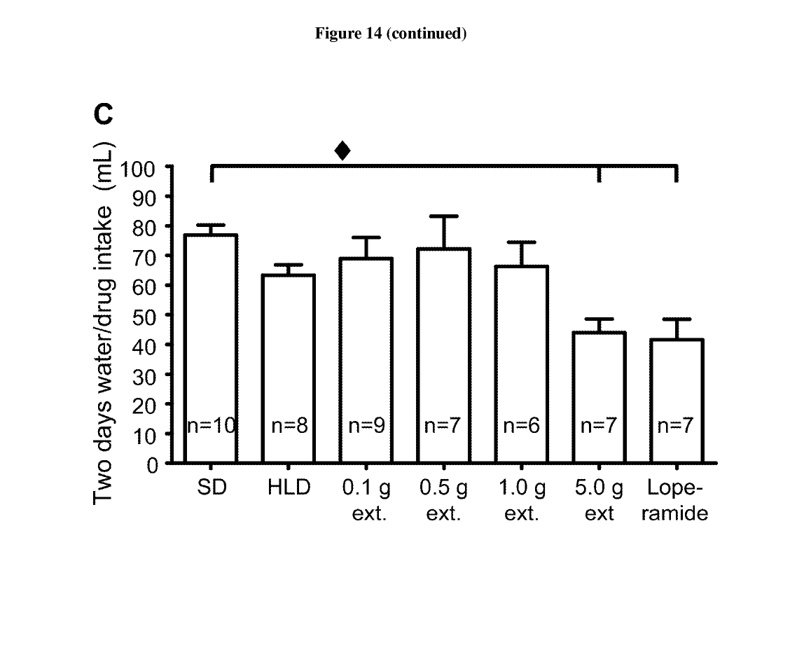

A HLD caused loss of weight (indicated by negative number values) in rats compared with rats fed a SO, while the SO fed rats actually gained weight (FIG. 14A; HLD: −3.12±0.58 g vs. SO: 7.50±0.50 g; P<0.001). Compared with rats on a HLD alone, the HLD rats treated with G. buchananii extract (0.1 g/L) gained weight (FIG. 14A; HLD: −3.12±0.58 g vs. GB: 0.82±0.4 g; P<0.05). Although not significant, G. buchananii extracts (0.5-1.0 g/L) showed a trend towards reversing the weight loss caused by ingesting a HLD (GB: −1.05±1.27 g and −70.78±0.81 g vs. HLD: −3.12±0.58 g; P>0.05, respectively). In contrast, rats treated with G. buchananii extract (5.0 g/L) lost weight with the same magnitude as rats on a HLD (GB: −3.20±1.87 g vs. HLD: −3.12±0.58 g; P>0.05). Loperamide showed a trend of reversing weight loss in the HLD rats by 1.78 g, which was not significant when compared with a HLD (LP: −1.33±0.64 g vs. HLD: −3.12±0.58 g; P>0.05). Although rats treated with a lower dose of G. buchananii (0.1 g/L) gained 2.15 g compared with rats treated with loperamide, the difference was not significant (GB: 0.82±0.4 g vs. LP: −1.33±0.64 g P>0.05). Taken together, these observations show that a HLD caused weight loss in rats, whilst G. buchananii (0.1 g/L) reversed this weight loss. Other lower doses of G. buchananii extract (0.5-1.0 g/L) showed considerable trends towards reversing weight loss.

Garcinia buchananii Extract Did not Improve Food Intake in Diarrheic Rats

A HLD reduced food intake in rats when compared with those on a SD (FIG. 14B; HLD: 34.39±1.08 g vs. SD: 41.37±1.59 g; P<0.01). There was no difference in food consumption between untreated HLD rats and rats treated with G. buchananii extract at doses of 0.1 g/L (HLD: 34.39±1.08 g vs. GB: 32.47±2.21 g; P>0.05), 0.5 g/L (HLD: 34.39±1.08 g vs. GB: 29.3±3.2 g; P>0.05), 1.0 g/L (HLD: 34.39±1.08 g vs. GB: 28.47±2.77 g; P>0.05) or 5.0 g/L (HLD: 34.39±1.08 g vs. GB: 27.94±2.67 g; P>0.05). Likewise, food consumption in the HLD rats was not improved by loperamide treatment (LP: 28.50±3.58 g vs. HLD: 34.39±1.08 g; P>0.05). There was also no significant difference in food intake between rats treated with loperamide and those treated G. buchananii extract (all doses; P>0.05). Taken together, these observations show that a HLD caused a significant decline in food intake. Garcinia buchananii extract and loperamide failed to improve food intake in rats with a HLD-induced diarrhea.

A HLD and 0.1-1.0 g/L G. buchananii Extract Did not Alter Water Consumption

Rats fed a SD consumed the same amount of water as those on a HLD (FIG. 14C; SD: 76.88±3.39 mL vs. HLD: 63.55±3.55 mL; P>0.05). No difference in water intake was apparent between rats on a HLD and rats treated with G. buchananii extract at dose of 0.1 g/L (HLD: 63.31±3.55 mL vs. GB: 68.93±7.12 mL; P>0.05), 0.5 g/L (HLD: 63.31±3.55 mL vs. GB: 72.25±10.95 mL; P>0.05), and 1.0 g/L (HLD: 63.55±3.55 mL vs. GB: 66.32±8.13 mL; P>0.05). However, G. buchananii extract at a dose of 5.0 g/L and loperamide decreased water consumption in the HLD rats when compared to the SD rats (G8: 44.00±4.57 mL and LP: 43.96±7.66 mL vs. SO: 76.88±3.39 mL; P<0.05 and P<0.01, respectively). In conclusion, at lower doses (0.1-1.0 g/L) G. buchananii extract did not significantly promote water intake in diarrheic rats. A higher dose (5.0 g/L) of extract and loperamide actually resulted in a decrease in water consumption.

The Anti-Motility Fractions PTLC1 and PTLC5 Treated Lactose-Induced Diarrhea and Reversed Weight Loss.

Rats treated with PTLC1 or PTLC5 recovered from diarrhea within 24 hours. One day after starting the treatments, rats treated with PTLC1 produced round, oblong and well-formed pellets. PTLC5 treated rats produced soft and poorly formed pellets, which gradually became transformed into well-formed and pliable fecal pellets after 2-3 days.

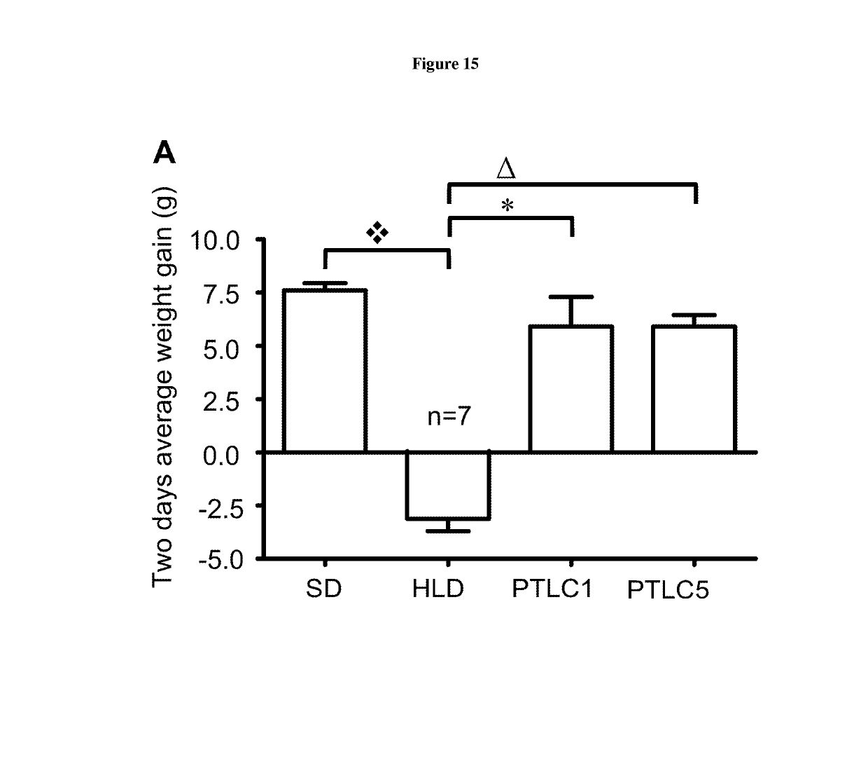

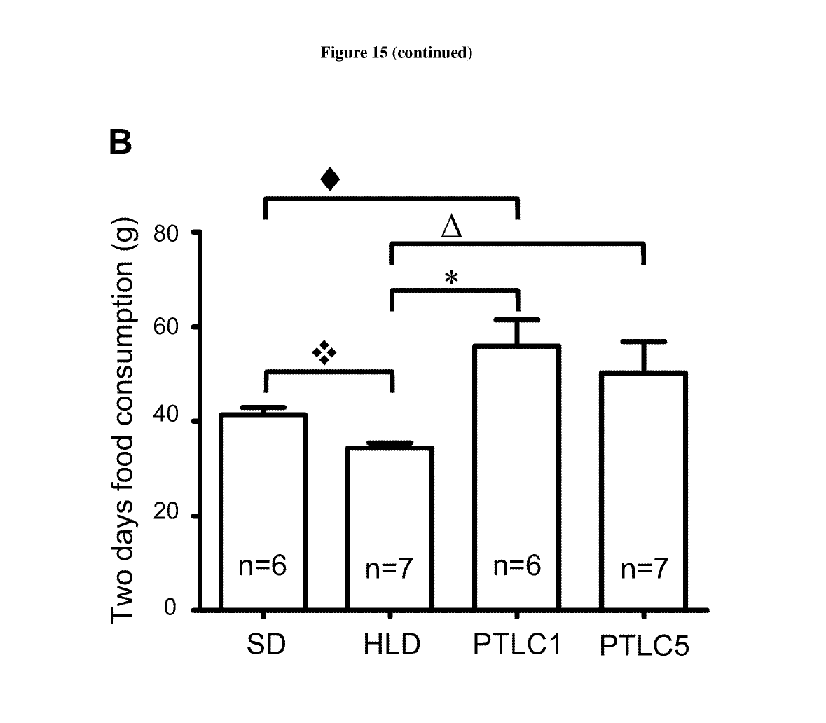

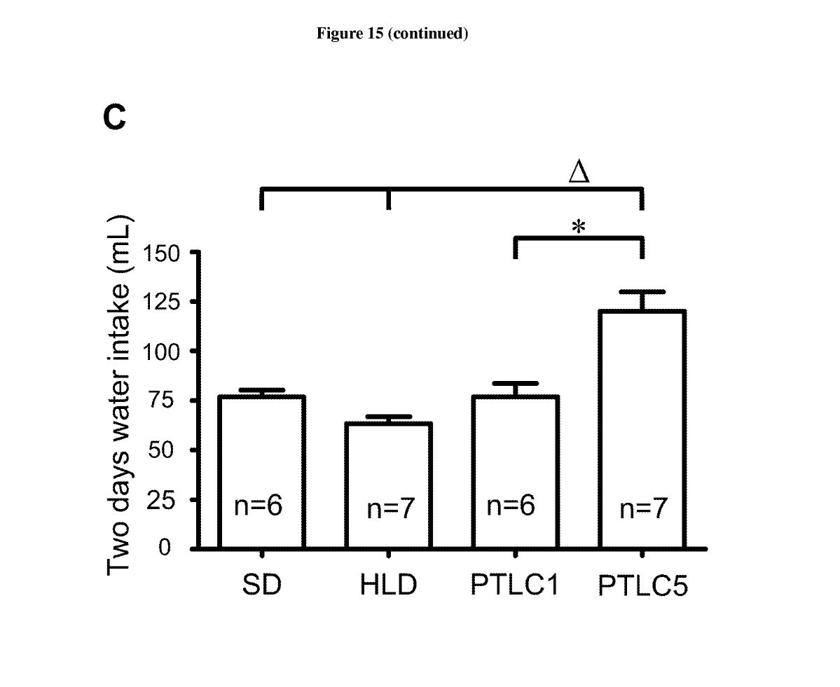

The transformation and hardening of diarrheic stools to form pellets was slower compared to the crude extract (0.5-5 g/L) in HLD rats treated with PTLC5. There was a complete reversal of weight loss in the HLD rats treated with PTLC1, when compared with the untreated HLD rats (FIG. 15A: PTLC1: 5.91±1.39 g vs. HLD: −3.12±0.58 g; P<0.001). Similarly, a complete reversal of weight loss was also apparent in diarrheic rats treated with PTLC5 (FIG. 15A: PTLC5: 5.91±0.54 g vs. −3.12±0.58 g; P<0.001), with no significant difference in magnitude of effect between PTLC1 and PTLC5 treatments (P>0.05). In summary, both PTLC1 and PTLC5 individually reversed the HLD induced weight loss, with a similar magnitude of effect observed with both treatments.

PTLC1 and PTLC5 Increased Food Consumption of Diarrheic Rats

Compared with untreated HLD rats there was a significant increase in food consumption in HLD rats treated with PTLC1 (FIG. 15B; HLD: 34.39±1.08 g vs. PTLC1: 55.93±5.56 g; P<0.001). Correspondingly, treating HLD rats with PTLC5 also significantly increased food consumption compared with untreated HLD rats (FIG. 15B; HLD: 34.39±1.08 g vs. PTLC5: 49.28±7.25 g; P<0.01). Interestingly, food intake of HLD rats treated with PTLC1 was significantly increased beyond that of SD rats (FIG. 15B; PTLC1: 55.93±5.56 g vs. SO: 41.37±1.59 g; P<0.01). There was no difference in food intake between rats treated with PTLC1 and PTLC5 (FIG. 15B; PTLC1: 55.93±5.56 g vs. PTLC5: 49.28 g±7.25; P>0.05) and between rats treated with PTLC5 and those on a SD (FIG. 15B; PTLC5: 49.28±7.25 g vs. SO: 41.37±1.59 g; P>0.05). In summary, both PTLC1 and PTLC5 significantly improved food consumption in HLD induced diarrheic rats, whilst PTLC1 also increased food intake above normal control levels.

PTLC5 Increased Fluid Consumption of Diarrheic Rats

There was no significant difference in fluid consumption between untreated HLD rats and HLD rats treated with PTLC1 (FIG. 15C; HLD: 63.31±3.55 mL vs. PTLC1: 76.96±6.82 mL; P>0.05). Fluid consumption in HLD rats treated with PTLC1 was almost identical to that of SD rats (FIG. 15C; PTLC1: 76.96±6.82 mL; vs. SD: 76.88±3.39 mL; P>0.05). Interestingly, treating HLD rats with PTLC5 significantly increased fluid consumption beyond that of SD rats (FIG. 15C; PTLC5: 120.20±9.83 mL vs. SD: 76.88±3.39 mL, P<0.001), untreated HLD rats (FIG. 15C; PTLC5: 120.20±9.83 mL vs. HLD: 63.31±3.55 mL, P<0.001) and HLD rats treated with PTLC1 (FIG. 15C; PTLC5: 120.20±9.83 mL vs. SD and PTLC1: 76.96±6.82 mL, P<0.01). In summary, PTLC5 markedly increased fluid intake in rats with HLD-induced diarrhea, whereas PTLC1 was without effect on fluid intake.

Gross Anatomical Observations of Intestine, Liver, Spleen and Kidney

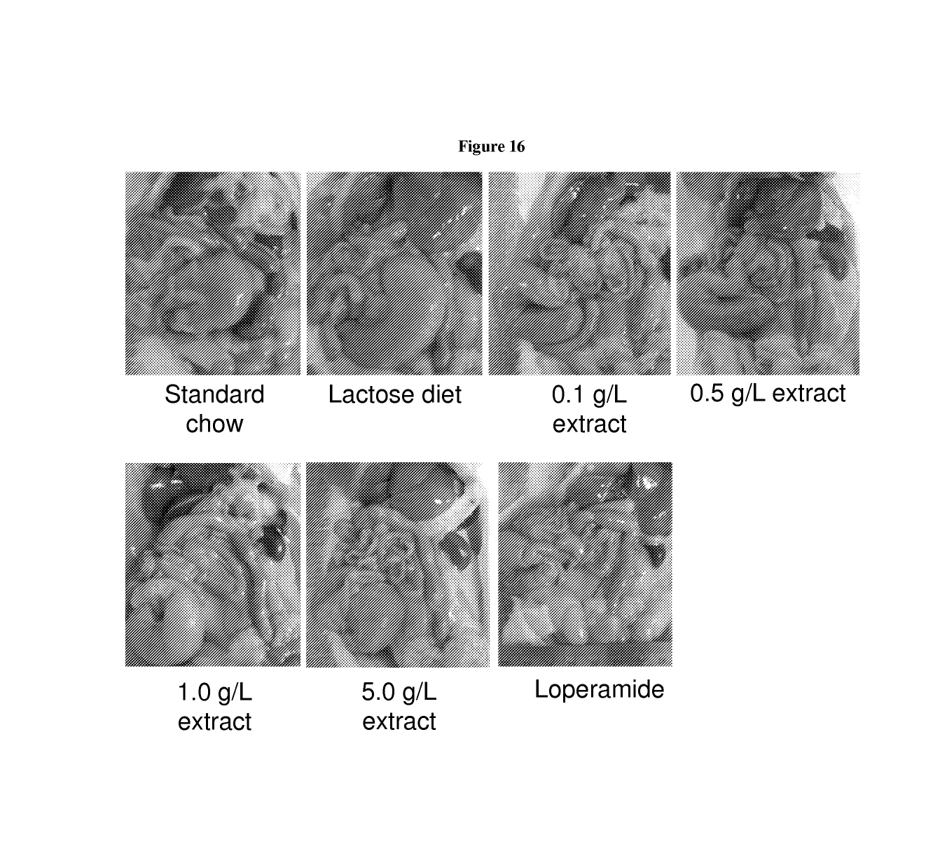

All SD rats had relatively small ceca and colons and less bloated with gas. Colons were filled with well-formed pellets (FIG. 16). Rats on a HLD had distended distal ileum, ceca and colons. The colons were often empty of stools but bloated with gas (FIG. 16).

HLD rats treated with G. buchananii extract (0.1-1.0 g/L) and loperamide also showed signs of distended ceca and colons but not as enlarged as those of untreated rats (cecum maximum diameter: 1.0 g/L: 2.1+1-0.3 cm vs. HLD: 2.9+1-0.5 cm). There were fewer well-formed pellets in the colons of rats treated with G. buchananii extract (0.1-0.5 g/L) as well as a relatively more bloated ceca and colons in comparison to those treated with G. buchananii extract (5.0 g/L). Rats treated with aqueous G. buchananii extract (1.0 g/L-5.0 g/L) had less bloated colons and produced relatively more well-formed fecal pellets in the colon (cecum maximum diameter: SD: 1.9+/−0.2 cm vs. HLD: 2.9+/−0.5 cm; P<0.01; HLD: 2.9+/−0.5 cm vs. 1.0 g/L extract: 2.1+/−0.3 cm; P<0.01 and HLD: 2.9+/−0.5 cm vs. LP: 2.0+/−0.2 cm; P<0.01; see FIGS. 13, 16). PTLC1 and PTLC5 also reduced bloating. None of the rats used in the experiment showed signs of hemorrhage or necrosis in the intestine, spleen, liver or kidney (FIG. 16). In summary, G. buchananii extract, its isolated fractions and loperamide all reduced bloating.

Discussion

The purpose of this study was to examine the effectiveness of G. buchananii extract and its fractions with anti-motility actions as a treatment against diarrhea using a model of lactose-induced diarrhea. We provide evidence that G. buchananii extracts and its anti-motility fractions are capable of treating the HLD-induced diarrhea, whilst significantly increasing food and fluid consumption and significantly increasing body mass. The efficacy of G. buchananii bark extract treatment when used at 1.0-5.0 g/L were in all cases highly comparable to those of loperamide, but actually outperformed loperamide in terms of weight gain or food/fluid intake. When used in small doses, G. buchananii extract (0.1 g/L) reversed the HLD diarrhea-induced weight loss.

This beneficial effect was augmented by both PTLC1 and PTLC5 individually. PTLC5 also significantly increased water intake. Garcinia buchananii extract and its fractions also reduced bloating. These new findings support the indigenous usage of the extract as a remedy for diarrheal diseases and the notion that G. buchananii extract holds great potential for the isolation of novel, non-opiate anti-diarrheal compounds. Garcinia buchananii bark extract stops diarrhea within 6-12 hours of treatment

Garcinia buchananii bark extract treats lactose-induced diarrhea within 6-12 hours

The ingestion of a HLD resulted in the onset of diarrhea in less than 24 hours. The signs of diarrhea and loss of body weight seen in this study correspond with findings from previous studies in which rats fed HLD had chronic diarrhea and became malnourished. A few animals with delayed onset diarrhea consumed less food indicating a correlation of disease severity with the amount of diet consumed. One of the new findings from this study was that G. buchananii extract, at all doses tested, treated lactose-induced diarrhea within 6-12 hours of initiating treatment. The overall effectiveness of 1.0-5.0 g/L G. buchananii extract in treating diarrhea was evidenced by the reduction of fecal fluid content leading to pellet production instead of stool. This effect was highly comparable to the effect of loperamide (8.4 mg/L). However, rats treated with loperamide produced up to four times fewer pellets compared with G. buchananii extract (1.0-5.0 g/L) treatment suggesting that loperamide caused a greater anti-motility effect than the extract. Fecal production in HLD rats treated with various does of the extract and loperamide did not return to normal SD values. The underlying reasons for this include feeding rats the HLD to maintain diarrhea, and a reduced food intake.

Garcinia buchananii extract reduced mucus and fecal fluid content. The extract caused the transformation of feces from a watery form to well-formed, pliable pellets suggesting that G. buchananii extract potentially has compounds that reduce intestinal mucus and fluid secretion, in addition to having compounds with anti-motility effects as shown in our previous studies. The excessive loss of body fluid occurs in all forms of diarrheal diseases and it is the main cause of debilitation and deaths.

We have shown for the first time, that G. buchananii extract causes a drastic decline in stool mucus and fluid content (fresh weight). This is associated with transformation of pellets from a soft loose form to normal pellets in less than 24 hours suggesting that G. buchananii extract prevents mucus secretion and loss of body fluids. Such a reduction of fecal mucus conforms with indigenous reports that an extract of the outer rind of the fruits of Garcinia indica is a folk remedy for dysentery and mucous diarrhea. Our observations suggest that G. buchananii contains compounds that reverse the increased mucus and fluid secretion associated with diarrhea due to lactose intolerance in rats. Whether the extract promotes intestinal fluid absorption needs to be confirmed by investigating the effect of the extract and its isolated compounds on mucosal fluid transport. Taken together, our observations suggesting that G. buchananii extract effectively reduced stool fluid content and bowel motility in a non-infectious model of diarrhea, sets the premise to test the extract against infectious diarrheas.

Lower Doses and Anti-Motility Fractions of G. buchananii Bark Extract Reverse Weight Loss Associated with Lactose Diarrhea-Induced.

Another interesting and novel finding of this study is that G. buchananii extract (0.1 g/L) and the anti-motility fractions, PTLC1 & PTLC5 reversed weight loss induced by a HLD. The considerable decrease in the weight of HLD rats indicates malnutrition, which is a key symptom of lactose-induced diarrhea in humans. In most incidences of diarrhea intestinal injury, altered mucosal function and loss of appetite results in malnutrition. Individuals with acute diarrhea consume less food, have reduced absorptive capacity than those who are healthy, which leads to the continuous decrease in weight. In most cases of chronic diarrhea and malnourished patients, the intestinal mucosa is damaged. Subsequently, patients become more prone to new and longer episodes of diarrhea, which exacerbates their nutritional status and health. Therefore, diarrhea in malnourished children and animals is difficult to treat and adequate nutrition is very critical to the treatment of diarrhea.

Our data strongly suggest that G. buchananii has the potential to promote weight gain both in lower doses and as purified components (PTLC1 and PTLC5). The reversal in weight loss observed in the present study could be due to a reduction of body fluid loss and an increase in food and fluid consumption (PTLC5). Whether G. buchananii extract and its derivatives could promote weight gain in humans when used as anti-diarrhea medications, and indeed the mechanisms involved are important questions that remain to be addressed. The exciting prospect is that G. buchananii bark extract may contain specific compounds that affect appetite. In support of this idea, the extract from the fruit rind of Garcinia indica is traditionally used to stimulate appetite. Our findings that G. buchananii serves as a effective anti-diarrheal remedy while promoting food and water consumption is extremely promising, as increased nutrition is critical to maintaining energy homeostasis during the rapid loss of fluids, essential electrolytes and nutrients in diarrheic patients. The effectiveness and advantages of the extract and its fractions highlights the need to determine the efficacy and safety of these preparations, especially as a treatment of diarrhea in children.

At a higher dose, G. buchananii extract did not alter HLD diarrhea-induced weight loss. Rather, there was a trend towards increasing weight loss. We speculate this could be attributable to (−)-hydroxycitric acid (HCA), a derivative of citric acid found in fruit rind form in Garcinia species. HCA is thought to cause weight loss by competitively inhibiting the enzyme adenosine triphospharease-citrate-lyase and increasing serotonin release or availability in the brain, which leads to suppression of appetite. If HCA is present in G. buchananii bark extract its effects may be become apparent when the extract is used at higher doses (5.0 g/L). Furthermore, the G. buchananii bark and its extracts have a bitter taste at increasing concentrations. As such it is possible that the bitter taste has an aversive effect for rats, reducing overall fluid consumption at the higher dose of the extract.

Garcinia buchananii Extract Reduces Intestinal Bloating

During most cases of lactose-induced diarrhea, there is an enlargement of the ileum, cecum and colon due to the accumulation of gas. 19 In humans, GI symptoms of lactose intolerance include pain, bloating, diarrhea and/or constipation and flatulence. All doses of crude G. buchananii extract, its fractions and loperamide reduced bloating in cecum and colon compared with untreated HLD rats. It has been shown that bloating in albino rats fed a HLD were associated with an increase in bacterial mass in the cecum, which may have also occurred in the HLD rats of the current study. However, it remains unclear whether these changes were due to the effect of drugs on intestinal bacteria or intestinal structure and physiology, or both. Whether the preparation from G. buchananii bark extract could benefit patients with symptoms of bloating and diarrhea such as patients with lactose- and food-intolerance, Irritable Bowel Syndrome needs to be tested.

Phytochemical analysis performed on G. buchananii extract and its fractions with anti-motility effects, PTLC1 and PTLC5 indicated the presence of flavonoids, tannins, alkaloids and steroids. These phytochemicals are considered to be responsible for the anti-diarrheal medicinal properties of most plant extracts. They act by suppressing gut motility, which delays gastrointestinal transit, the common feature of botanical extracts with anti-diarrheal properties. It appears evident that flavonoids, tannins, alkaloids and steroids are likely to be responsible for the antidiarrheal effects of G. buchananii extract, either singly or in various combinations.

Conclusion:

This study shows that Garcinia buchananii is an effective antidiarrheal remedy, with an efficacy comparable to loperamide. The complete reversal of weight loss, increase in food and fluid consumption are additional benefits that are required to effectively treat diarrhea. There is need to establish the exact bioactive compounds and the mechanisms underlying the anti-diarrheal effects of G. buchananii in order to better understand how the extract works and lay the basis to promote its use for broader human treatment.

### Example 6

### Isolation and Structural Characterization of Compounds from G. buchananii Fractions

### Materials and Methods

Chemicals.

The following reagents were obtained commercially: hydrogen peroxide (Merck, Hohenbrunn, Germany); 2,2′-azino-bis(3-ethylbenzothiazoline-6-sulfonic acid) diammonium salt (ABTS), peroxidase from horseradish (HRP), (±)-6-hydroxy-2,5,7,8-tetramethylchromane-2-carboxylic acid (Trolox), Fluorescein sodium salt (FL), 2,2′-azobis(2-methylpropinamidine) (AAPH), quercetin, (−)-epicatechin, (±)-naringenin, methyliodine, ascorbic acid (Sigma-Aldrich, Steinheim, Germany), rutin, (+)-(2R,3R)-taxifolin (AppliChem, Darmstadt, Germany). Water for chromatographic separations was purified with a Milli-Q Gradient A10 system (Millipore, Schwalbach, Germany), and solvents used were of HPLC-grade (Merck, Darmstadt, Germany). Deuterated solvents were obtained from Euriso-Top (Gif-sur-Yvette, France).

General Experimental Procedure.

1D and 2D NMR spectroscopy 1H, 1H-1H-gCOSY, gHSQC, gHMBC, 13C, 1H-1H rotating frame nuclear Overhauser enhancement spectroscopy (phase-sensitive ROESY) NMR measurements were performed on an Avance III 500 MHz spectrometer with a CTCI probe or an Avance III 400 MHz spectrometer with a BBO probe (Bruker, Rheinstetten, Germany). Mass spectra of the compounds were measured on a Waters Synapt G2 HDMS mass spectrometer (Waters, Manchester, UK) coupled to an Acquity UPLC core system (Waters, Milford, Mass., USA). For CD spectroscopy, methanolic solutions of the samples were analyzed by means of a Jasco J810 Spectro polarimeter (Hachioji, Japan). HPLC separations were performed using a preparative HPLC system (PrepStar, Varian, Darmstadt, Germany). MPLC separations were performed on a Man Sepacore® (Flawil, Switzerland) system using PP cartridges (id. mm, 1.150 mm) and LiChroprep® 19 RP18, 25-40 um mesh material (Merck, Darmstadt, Germany).

Plant Material.

Garcinia buchananii stem bark was collected from plants in their natural habitats in Karagwe Tanzania, and processed as described above. A sample of bark powder was deposited at the University of Idaho Stillinger herbarium (voucher #159918).

Extraction and Isolation.

G. buchananii bark 1 powder (10 g) was suspended in a mixture of ethanol/water (50 mL, 70:30), sonicated (10 minutes), stirred at RT (20 min), and filtered. The filtrate was extracted with hexane (50 mL), the ethanol/water extract was evaporated and then the sample was freeze-dried. Aliquots (1 g) of the freeze-dried ethanol/water extract were dissolved in a water/methanol mixture (10 mL, 50:50, v/v) and fractionated using MPLC. Chromatography was performed starting with a mixture (65/35, v/v) of aqueous formic acid (0.1% in water, pH 2.5) and MeOH increasing the MeOH content up to 55% within 25 minutes, and in 5 min to 100%. Eight fractions (M1-M8, 160, 70, 401, 63, 101, 51, 51 and 52 mg, Table 1) were collected, concentrated under reduced pressure and freeze dried.

Antioxidant Assays

Hydrogen Peroxide Scavenging Assay.

Hydrogen peroxide scavenging assay was performed in accordance with the method of Ichikawa, M. and colleagues (2002). Sample solutions at appropriate concentration were prepared using phosphate buffer (100 mM, pH 6.0). Sample solution (100 mL), phosphate buffer (30 mL, 100 mM, pH 6.0), and hydrogen peroxide solution (10 mL, 500 mM) were mixed in 96-well clear microplate (VWR, Ismaning, Germany). Then peroxidase (40 mL, 150 U/mL) and ABTS (40 mL, 0.1%) were added. The microplate was incubated at 37° C. for 15 min. The absorbance (A) of each well was measured at 414 nm with FLUOstar OPTIMA (BMG LABTECH, Offenburg, Germany). The scavenging effect (E) was calculated as shown using the formula below (blank stands for solution without hydrogen peroxide, and control did not include a test compound) and EC50 was calculated by the probit method. After freeze-drying three times 1 MPLC fractions M1-M8 were analyzed in natural concentrations.

E=[(A−Ablank)control−(A−Ablank)test]/(A−Ablank)control·100

Oxygen Radical Absorbance Capacity (ORAC) Assay.

ORAC assay was carried out according to the method of Ou, G. and colleagues (1993) with some modifications. Trolox and FL were used as a standard and a fluorescent probe respectively. Free radicals were produced by AAPH to oxidize FL. Different dilutions of Trolox (200, 100, 50, 25, 12.5 mM) and appropriate dilutions of tested sample were prepared with phosphate buffer (10 mM, pH 7.4). Trolox dilution (25 mL) or sample solution were pipetted into a well of 96-well black microplate (VWR) and then FL (150 mL, 10 nM) was added. The reaction mixture was incubated at 37° C. for 30 min Afterward s, fluorescence was measured every 90 sec at the excitation of 485 nm and the emission of 520 nm using FLUOstar OPTIMA. After 3 cycles, AAPH (25 mL, 240 mM) was added quickly, and then the measurement was resumed and continued up to 90 min (60 cycles in total). The background signal was determined using the first 3 cycles. The ORAC values were calculated according to the method of Ou, G. and colleagues (1993) and expressed as Trolox equivalent (mmol TE/mmol). After freeze-drying in triplicate, MPLC fractions M1-M8 were analyzed in natural concentrations.

Isolation and Structural Characterization of Compounds with Antioxidant Activity.

Fractions that showed higher levels of antioxidant activities were subjected to the identification and characterization of chemical compounds.

M3 afforded (2R,3R,2″R,3″R)-manniflavone (3, FIG. 17), and

MPLC fractions (M1 and M4-M5) were further purified by means of HPLC. M1: Chromatography was performed using a RP column (21.2, 250 mm, Phenylhexyl, 5 um; Phenomenex, Aschaffenburg, Germany) as the stationary phase. The effluent (18 mL/min) was monitored at 290 nm. The separation started with a mixture (83/17, v/v) of aqueous formic acid (0.1% in water, pH 2.5) and MeOH, and the MeOH content was increased up to 40% within 12 minutes. Collected fractions were concentrated under reduced pressure and freeze-dried twice, affording (2R,3R)-taxifolin-6-C-β-D-glucopyranoside (1, FIG. 17) and (2R,3R)-aromadendrin-6-C-β-D-glucopyranoside (2, FIG. 17). M4: Using the same column and flow rate as described above, chromatography was performed starting with a mixture (70/30, v/v) of aqueous formic acid (0.1% in water, pH 2.5) and ACN, and increasing the ACN content up to 43% within 13 minutes. The collected fraction was concentrated under reduced pressure and freeze-dried twice, affording (2R,3R,2″R,3″R) GB-2 (4, FIG. 17). M5: Using the same column and flow rate as described above, chromatography was performed starting with a mixture (65/32, v/v) of aqueous formic acid (0.1% in water, pH 2.5) and ACN, and increasing the ACN content up to 43% within 13 minutes. The collected fraction was concentrated under reduced pressure and freeze-dried twice, affording (2R,3S,2″S) buchananiflavanone (5, FIG. 17).

Methylation of Manniflavone.

Manniflavone (3) (100 mg) was dissolved in dry acetone (100 mL), and methyliodine (2 mL) and K₂CO₃, (2 g) added. The mixture was refluxed for 24 hr, with the addition of additional methyliodine (0.5 mL) and K₂CO₃, (1 g) after 8 hr. The mixture was evaporated to dryness and then taken up in a water/methanol mixture (2 mL, 50:50, 1 v/v) and purified by solid phase extraction (SPE) (Strata Gigatube C18, Phenomenex, Aschaffenburg, Germany). The SPE cartridge was flushed with water and the methanol eluate was further purified by means of HPLC. Monitoring the effluent (4.2 mL/min) at 290 nm, chromatography was performed using a RP column 10×250 mm, Phenylhexyl, 5 um (Phenomenex, Aschaffenburg, Germany) as the stationary phase and starting with a mixture (20/80, v/v) of aqueous formic acid (0.1% in water, pH 2.5) and MeOH. The MeOH content was increased to 100% within 20 minutes. Collected fractions were concentrated under reduced pressure and freeze-dried twice, affording (2R,3R,2″R,3″R)-Nonamethylmanniflavone (3a) and a mixture of two octamethylmanniflavanones (3b,c).

(2R,3R)-Taxifolin-6-C-β-D-glucopyranoside (1, FIG. 17): colorless powder; UV (MeOH/H₂O, 5/5, v/v) λₘₐₓ=225, 290, 345 nm; (−) HRESIMS m/z 465.1035 [M-H]. (calcd for C2+121012, 465.1033); CD (MeOH, 0.67 mmol/L): λₘₐₓ (Δε)=333 (+2.0), 296 (−6.5), 252 (+0.9), 222 (+6.6); ¹H NMR (500 MHz, DMSO-d₆, COSY): 3.10 [m, 1H, J=9.0 Hz, H—C(4″)], 3.14 [m, 1H, J=6.0 Hz, H(5″)], 3.16 [dd, 1H, J=8.4, 8.6 Hz H—C(3″)], 3.40 [d, 1H, J=11.1 Hz, H—C(6α″)], 3.67 [d, 1H, J=11.1 Hz, H—C(6β″)], 3.99 [pt, 1H, J=9.1 Hz, H—C(2″)], 4.45 [dd, 1H, J=5.3 Hz, H—C(3)], 4.49 [d, 1H, J=9.8 Hz, H—C(1″)], 4.97 [d, 1H, J=10.8 Hz, H—C(2)], 5.74 [d, 1H, J=5.7 Hz, HO—C(3)], 5.92 [s, 1H, H—C(8)], 6.74 [s, 2H, H—C(5′,6′)], 6.87 [s, 1H, H—C(2′)], 8.98 [2×brs, 2H, HO—C(3′,4′)], 12.47 [s, 1H, HO—C(5)]; ¹³C NMR (125 MHz, DMSO-d₆, HSQC, HMBC): δ 61.6 [C-6″], 70.3 [C-2″], 70.7 [C-4″], 71.6 [C-3], 73.0 [C-1″], 79.1 [C-3″], 81.6 [C-5″], 82.9 [C-2], 94.7 [C-8], 100.1 [C-4a], 106.0 [C-6], 115.2 [C-5′], 115.3 [C-2′], 119.3 [C-6′], 128.0 [C-1′], 145.0 [C-4′], 145.8 [C-3] 161.3 [1 C-8a], 162.6 [C-5], 166.1 [C-7], 197.9 [C-4].

(2R,3R)-Aromadendrin-6-C-β-D-glucopyranoside (2, FIG. 17): colorless powder; UV (MeOH/H₂O, 5/5, v/v) λₘₐₓ=213, 228, 293, 347 nm; (−) HRESIMS m/z 449.1100 [M-H]− (calcd for C₂₁H₂₁O₁₁, 449.1084)); CD (MeOH, 0.74 mmol/L): λₘₐₓ (Δε)=329 (+1.5), 291 (−3.8), 248 (+1.3), 233 (+3.2), 218 (+7.0); ¹H NMR (500 MHz, DMSO-d₆, COSY): 3.11 [m, 1H, J=9.1 Hz, H—C(4″)], 3.12 [m, 1H, H—C(5″)], 3.16 [pt, 1H, J=8.4, 8.7 Hz H—C(3″)], 3.41 [1H, HC (6α″)], 3.66 [d, 1H, J=10.7 Hz, H—C(6β″)], 4.00 [pt, 1H, J=9.2 Hz, H—C(2″)], 4.48 [d, 1H, J=9.8 Hz, H—C(1″)], 4.50 [d, 1H, J=11.0 Hz, H—C(3)], 4.99 [d, 1H, J=11.0 Hz, H—C(2)], 5.74 [brs, HO—C(3)], 5.83 [s, 1H, H—C(8)], 6.78 [d, 2H, J=8.6 Hz, H—C(3′,5′)], 7.29 [d, 2H, J=8.6 Hz, H—C(2′,6′)], 9.57 [brs, 1H, HOC (4′)], 12.50 [brs, 1H, HO—C(5)]; ¹³C NMR (125 MHz, DMSO-d₆, HSQC, HMBC): δ 61.5 [C-6″], 70.3 [C-2″], 70.6 [C-4″], 71.4 [C-3], 73.1 [C-1″], 79.1 [C-3″], 81.4 [C-5″], 82.7 [C-2], 95.2 [C-8], 99.5 [C-4a], 106.2 [C-6], 114.9 [C-3′,5′], 127.6 [C-1′], 129.3 [C-2′,6′], 157.7 [C-4′], 161.2 [C-8a], 162.7 [C-5], 167.5 [C-7], 197.0 [C-4].

(2R,3S,2″R,3″R)-manniflavanone (3, FIG. 17): colorless powder; UV (MeOH/H₂O, 6/4, v/v) λₘₐₓ=210, 290, 346 nm; (−) HRESIMS m/z 589.0989 [M-H]− (calcd for C₃₀H₂₁O₁₃, 589.0982); CD (MeOH, 0.46 mmol/L): λₘₐₓ (Δε)=341 (+1.3), 321 (−0.7), 303 (−5.6), 283 (+7.6), 240 (−1.3), 218 (−11.7); ¹H NMR (400 MHz, Acetone-d₆+DMSO-d₆, 9/1, 8° C., COSY): δ 4.05 [dd, J=5.4, 11.7 Hz, H—C(3″)], 4.30 [dd, J=5.4, 11.3 Hz, H—C(3″)], 4.55 [d, J=12.1, H—C(3)], 4.71 [d, J=12.1, H—C(3)], 4.91 [d, J=11.7 Hz, H—C(2″)], 5.09 [d, J=11.3 Hz, H—C(2″)], 5.45 [d, J=12.1 Hz, H—C(2)], 5.64 [d, J=5.9 Hz, HO—C(3″)], 5.68 [d, J=12.1 Hz, H—C(2)], 5.80 [d, J=5.9 Hz, HO—C(3″)], 5.81-5.94 [4×s, H—C(6″,1 6,8)], 6.03 [s, H—C(6″)], 6.60 [dd, J=1.8, 7.9 Hz, H—C(6′)], 6.66-6.71 [m, H—C(6′,5′,5′″)], 6.77-6.80 [2×dd, J=1.8, 8.4 Hz, H—C(6″)], 6.83-6.89 [m, H—C(5′,5′″,2′)], 6.89-6.94 [m, H—C(2′,2′″)], 8.80-9.00 [4×brs, HO—C(3′,4′,3′″, 4″)], 10.64, 10.68, 11.00, 11.24 [brs, HO—C(7″,7)], 11.89, 11.97 [s, HO—C(5″)], 12.29, 12.36 [s, HO—C(5)]; ¹³C NMR (100 MHz, Acetone-d₆+DMSO-d₆, 9/1, HSQC, HMBC): δ 48.2 [C-3], 73.0, 73.3 [C-3″], 82.3, 82.7 [C-2], 83.8 [C-2″], 95.4, 95.5, 96.1, 96.4, 96.6 [C-6″,6,8], 100.4, 101.0 [C-4a″], 101.9, 102.0 [C-4a,8″], 115.4, 115.5, 115.6, 115.9, 116.1 [0-5′″,5′,2′″,2′], 118.1 [0-6′″], 119.2, 119.4, 119.5 [C-6′″,6′,6′], 129.0, 129.1, 129.3, 129.5 [C-1′,1′″], 145.4, 145.5, 146.2, 146.3, 146.4, 146.5 [C-3′,4′,3′″,4′″], 160.4, 161.1 [C-8a″], 162.9, 163.2 [C-5″], 163.5, 163.6 [C-8a], 164.6, 164.7 [C-5], 165.4, 166.0 [C-7″], 167.2, 167.3 [C-7], 197.3, 197.5 [C-4], 198.2 [C-4″].

(2R,3S,2″R,3″R)-GB-2 (4, FIG. 17): colorless powder; UV (MeOH/H₂O, 6/4, v/v) λₘₐₓ=204, 292, 347 nm; (−) HRESIMS m/z 573.1037 [M-H]-(calcd for C₃₀H₂₁O₁₂, 573.1033); CD (MeOH, 0.47 mmol/L): λₘₐₓ (Δε)=341 (+2.0), 320 (−1.6), 303 (−9.1), 281 (+12.0), 246 (−2.3), 237 (0.0), 214 (−16.4); 1H NMR (400 MHz, Acetone-d₆+DMSO-d₆, 9/1, 8° C., COSY): δ 4.10 [dd, J=4.9, 11.7 Hz, H—C(3″)], 4.31 [dd, J=4.7, 11.2 Hz, H—C(3″)], 4.61 [d, J=12.2, H—C(3)], 4.77 [d, J=12.2, H—C(3)], 4.93 [d, J=11.7 Hz, H—C(2″)], 5.06 [d, J=11.2 Hz, H—C(2″)], 5.43 [m, HO—C(3″)], 5.53 [d, J=12.2 Hz, H—C(2)], 5.57 [m, HOC (3″)], 5.77 [d, J=12.2 Hz, H—C(2)], 5.82 [s, H—C(6″)], 5.91-6.03 [4×s, HC (6,8)], 6.73 [m, H—C(3′,5′)], 6.79 [d, J=8.0 Hz, H—C(6″)], 6.86-6.90 [m, HC (5′″)], 6.92 [s, H—C(2″)], 7.01 [d, J=1.5 Hz, H—C(2″)], 7.21 [m, J=8.9 Hz, HC (2′,6′)], 8.63-8.86 [4×brs, HO—C(3′″, 4″)], 9.33, 9.40 [brs, HO—C(4′)], 10.57, 10.91, 11.19 [brs, HO—C(7″,7)], 11.84, 11.92 [s, HO—C(5″)], 1 12.32, 12.39 [s, HO—C(5)]; ¹³C NMR (100 MHz, Acetone-d₆+DMSO-d₆, 9/1, HSQC, HMBC): δ 48.1 [C-3], 72.8, 73.3 [C-3″], 82.1, 82.5 [C-2], 83.8 [C-2″], 95.3, 95.4, 96.0, 96.4, 96.5 [C-6″,6,8], 100.3, 100.8 [C-4a″], 101.9 [C-4a], 101.8, 102.0 [C-8″], 115.2, 115.3 [C-3′,5′], 115.4, 115.5 [C-5′″], 115.6, 115.7 [C-2′″], 118.1, 119.8 [C-6′″], 128.4, 128.5 [C-1′″], 128.6, 128.8 [C-1′], 129.1 [C-2′,6′], 145.2, 145.6, 146.0, 146.5 [C-3′″,4′″], 158.5, 158.6 [C-4′], 160.3, 161.0 [C-8a″], 163.0, 163.3 [C-5″], 163.5, 163.6 [C-8a], 164.7, 164.8 [C-5], 165.5, 166.0 [C-7″], 167.1, 167.2 [C-7], 197.3, 197.4 [C-4], 198.2 [C-4″].

(2R,3S,2″S)-Buchananiflavanone, (2R,3S,2″S)-2-(3,4-dihydroxyphenyl)-2,2′,3,3′-tetrahydro-5,5,7,7-tetrahydroxy-2′-(3,4-dihydroxyphenyl)-[3,8′-Bi-4H-1-benzopyran]-4,4′-dione (5, FIG. 17): colorless powder; UV (MeOH/H₂O, 6/4, v/v) λₘₐₓ=215, 225, 291, 347 nm; (−) HRESIMS m/z 573.1037 [M-H]− (calcd for C₃₀H₂₁O₁₂, 573.1033); CD (MeOH, 0.37 mmol/L): λₘₐₓ (Δε)=341 (+1.5), 315 (−1.2), 298 (−5.6), 282 (+5.8), 239 (−2.5), 218 (−13.1); ¹H NMR (400 MHz, DMSO-d₆, COSY): δ 8 2.60 [dd, J=15.2 Hz, HC (3α″)], 2.69 [2×dd, J=14.7 Hz, H—C(3αβ″)], 2.96 [dd, J=13.6, 13.8 Hz, HC (3β″)], 4.50 [d, J=12.1, H—C(3)], 4.63 [d, J=12.0, H—C(3)], 5.29 [d, J=12.1 Hz, H—C(2″)], 5.39 [d, J=12.2 Hz, H—C(2)], 5.42 [d, J=12.1 Hz, H—C(2″)], 5.64 [d, J=12.0 Hz, H—C(2)], 5.80, 5.87, 5.90 [3×s, H—C(6,8,6″)], 6.49 [d, J=12.1 Hz, HC (6′)], 6.58-6.76 [m, H—C(5′,5′″,2′,2′″)], 6.62 [m, H—C(6″)], 6.63 [m, H—C(6′)], 6.71 [m, H—C(6″)], 6.81 [m, J=7.7 Hz, H—C(2′)], 6.83 [m, J=7.7 Hz, H—C(2′″)], 8.93-9.00 [brs, HO—C(3′,4′,3′″,4″)], 10.83 [brs, HO—C(7″,7)], 12.05, 12.15 [s, HO—C(5″)], 12.17, 12.21 [s, HO—C(5)]; ¹³C NMR (100 MHz, DMSO-d₆, HSQC, HMBC): δ 43.1 [C-3″], 47.4 [C-3], 78.5, 78.6 [C-2″], 81.6, 81.9 [C-2], 94.9, 95.0, 95.2, 95.5, 96.1 [C-6″,6,8], 101.1, 101.2 [C-4a,4a″], 101.6 1 [C-8″], 113.4 [C-2′], 114.0 [C-2′″], 114.7, 114.9, 115.1, 115.3, 115.6, 115.7 [C-2′,5′,2′″,5′″], 116.4, 117.2 [C-6′″], 118.5, 118.9 [C-6′″], 128.5, [C-1′], 129.9 [C-1′″], 144.7, 145.1, 145.3, 145.5, 145.6, 145.7 [C-3′,4′,3′“,4′″], 159.9, 160.8 [C-8a″], 162.0, 162.4 [C-5″], 162.6, 162.8 [C-8a], 163.6, 163.7 [C-5], 164.6, 165.0 [C-7″], 166.3, 166.4 [C-7], 196.1 [C-4″], 196.6 [C-4].

7 (2R,3R,2″R,3″R)-Nonamethylmanniflavanone (3a): slight brownish powder; UV (MeOH/H₂O, 9/1, v/v) λₘₐₓ=210, 290, 346 nm; (−) HRESIMS m/z 715.2390 [M-H]− (calcd for C₃₉H₃₉O₁₃, 715.2391) ¹H NMR (500 MHz, CDCl₃, COSY): δ 3.70-3.93 [9×s, CH₃O—C(5,7,3′,4′,3″,5″,7″,3′″,4′″)], 4.48 [d, 1H, J=12.5, H—C(3)], 4.73 [d, 1H, J=12.6, H—C(3″)], 5.65 [d, 1H, J=12.4 Hz, HC (2)], 5.83 [d, 1H, J=12.7 Hz, H—C(2″)], 6.15-6.19 [3×s, 3H, H—C(6,8,6″)], 6.56 [d, J=7.9 Hz, H—C(6′)], 6.61-6.91 [H—C(2′,5′,2′″,5′″)], 6.91 [d, J=8.2 Hz, HC (6′″)] 130 NMR (125 MHz, CDCl₃, HSQC, HMBC): δ 49.1 [0-3″], 51.2 [C-3], 55.6-56.3 [CH₃O-(5,7,3′,4′,3″,5″,7″,3′″,4′″)], 81.4 [C-2″], 81.6 [C-2], 93.2-93.6 [C-6″,6,8], 100.7, 100.8 [C-4a″,4a], 105.8 [0-8″], 109.4-110.8 [C-2′,5′,2′″,5′″], 119.8 [C-6′], 120.2 [C-6′″], 130.4 [C-1′″], 130.7 [C-1′], 147.7-149.4 [C-3′,4′,3′″,4′″], 162.5-170.1 [C-5,7,8a,8a″,5″,7″], 188.0 [C-4], 188.3 [0-4″].

Octamethylmanniflavanones (3b,c): slight brownish powders; UV (MeOH/H₂O, 9/1, v/v) λₘₐₓ=210, 290, 346 nm; (−) HRESIMS m/z 701.2225 [M-H]-(calcd for C₃₈H₃₇O₁₃, 701.2234), ¹³C NMR (125 MHz, CDCl₃): δ 55.5-56.1 [CH₃O-(5,7,3′,4′,3″,5″,7″3′″,4′″)].

(+)-(2R,3R)-Taxifolin (6, FIG. 17): CD (MeOH): λₘₐₓ (Δε)=330 (+2.5), 295 (−10.0), 252 (+2.0), 244 (+1.7), 222 (+10.6), 208 (−4.7).

### Results

In a first antioxidative screening, the aqueous ethanolic extract of Garcinia buchananii and MPLC fractions thereof was analyzed by means of ORAC and H₂O₂ assays (Table 5).

TABLE 5
Antioxidant activities of Garcinia buchananii extract and
fractions M1-M8.

| | | | | ORAC assay^{3,4} | |
|---|---|---|---|---|---|
| | | H₂O₂ assay^{1,2} | | (μmol TE/each | |
| | Each amount (mg) | EC₅₀ (dilution degree) | | amount) | |
| EtOH extract | 100.0 | 11320.5 | (10061.5-12721.7) | 1359.15 | (±14.84) |
| Recombination of M1-M8 | | 8912.8 | (7840.1-10040.1) | 1102.95 | (±46.09) |
| Calculated sum of M1-M8 | | 9879.8 | | 1251.78 | |
| M1 | 16.3 | 809.4 | (534.1-702.0) | 102.38 | (±3.05) |
| M2 | 7.1 | 673.1 | (600.6-748.9) | 80.89 | (±1.38) |
| M3 | 41.0 | 5879.3 | (5008.0-6473.8) | 744.83 | (±27.87) |
| M4 | 6.4 | 722.3 | (639.9-812.1) | 86.89 | (±1.61) |
| M5 | 10.3 | 1039.1 | (894.4-1207.7) | 141.68 | (±3.44) |
| M6 | 5.2 | 382.4 | (331.2-444.7) | 44.89 | (±1.58) |
| M7 | 8.3 | 572.2 | (509.9-641.2) | 42.34 | (±2.97) |
| M8 | 5.3 | 202.1 | (180.5-226.4) | 7.89 | (±0.20) |

¹Each sample was analyzed by means of H₂O₂ assay by triplicate studies.
²Range in brackets represents 95% confidence interval.
³Each sample was analyzed by means of ORAC assay by quadruplicate studies.
⁴Numerical value in brackets represents SD.

The ethanolic extract of Garcinia buchananii revealed an extraordinary high antioxidant value of 1359 μmol TE/100 mg. This is higher than or comparable to that of natural product extracts known to have high antioxidative activities, such as bilberry, elderberry, red wine extract, and grape seed extract. These have ORAC values of 265, 222, 694 and 1189 μmol TE/100 mg, respectively.

To get a deeper insight into the chemistry of this highly antioxidative aqueous ethanolic extract of Garcinia buchananii, as well as to focus on the highly antioxidative compounds, the sample was fractionated using medium pressure liquid RP-18 chromatography. Eight fractions (M1-M8) were obtained and then analyzed in their natural ratios for antioxidant activities using the ORAC and H₂O₂ scavenging assays. By far the highest antioxidant activity was observed both in the ORAC and hydrogen peroxide scavenging assays for fraction M3. In both cases, the activity of this fraction represented about the half of the whole activity of the crude extract (Table 5). This was followed by fraction M5, which showed the second highest activity in both assays, and by fractions M1, M2, and M4, which all had a similar range of antioxidative activities. Fractions M6-M8 had the lowest activities. Therefore, fractions M1-M5 were further purified by means of RP-HPLC as described above.

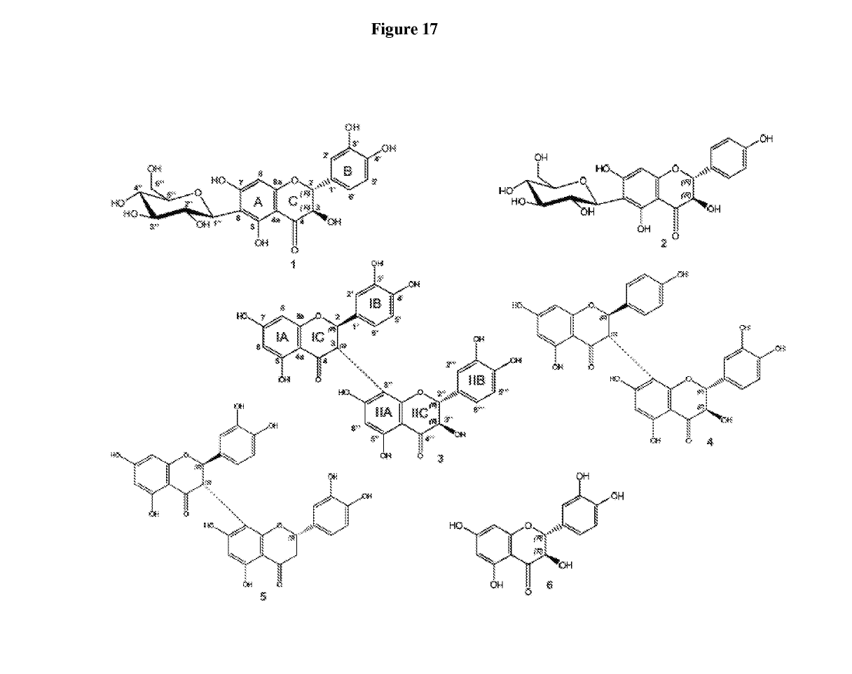

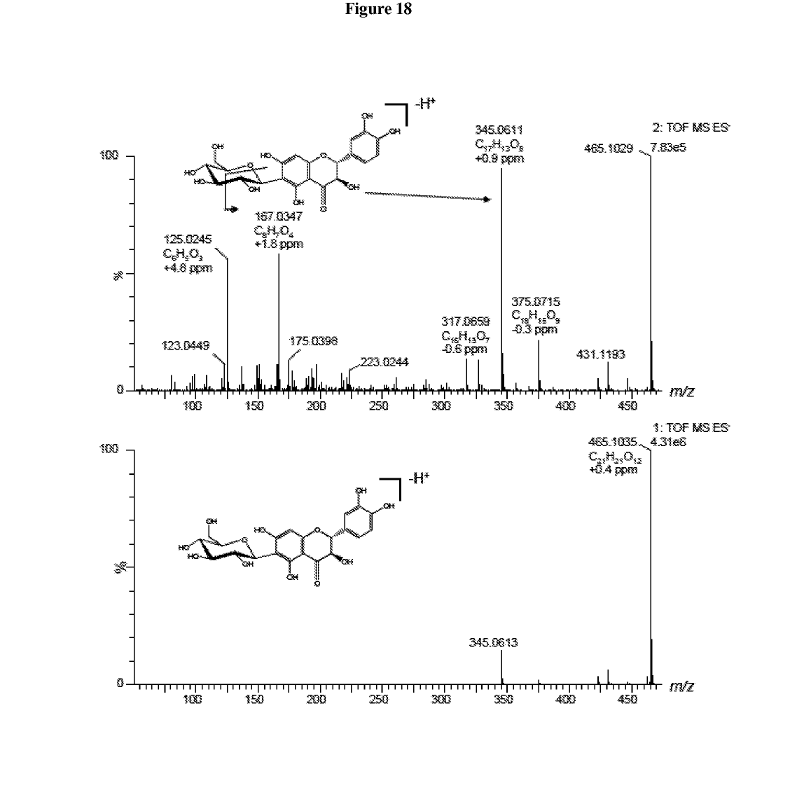

The antioxidative compound no. 1 isolated from fraction M1 was obtained as a colorless amorphous powder. This compound showed the typical absorption maxima expected for flavonoids. Results from electrospray ionization (ESI) MS indicated this compound forms an [M-H]⁻ ion with m/z 465, as well as a fragment ion with m/z 345, as expected for a C-glycoside. As shown in FIG. 18, high resolution LC-MS analysis confirmed the target compound to have the molecular formula C₂₁H₂₂O₁₂ and the fingerprint fragment C₁₇H₁₄O₁₈. The ¹H NMR spectrum of compound 1 showed an aromatic singlet for H—C(8) at 5.92 ppm, three aromatic protons resonating at 6.74 and 6.87 ppm showing an ABX coupling system. In addition, the two aliphatic protons H—C(3) and H—C(2) were observed coupling with each other at 4.45 and 4.97 ppm suggesting a taxifolin aglycone. Besides the signals of the flavanon aglycone, the ¹H NMR spectrum also exhibited seven aliphatic protons resonating at 3.10 [H—C(4″), 3.14 ppm [H—C(5″)], 3.16 ppm [H—C(3″)], 3.40 ppm [H—C(6a″)], 3.67 ppm [H—C(6b″)], 3.99 ppm [H—C(2″)], and 4.49 ppm [H—C(1″)] as expected for a hexose unit.

Considering all the coupling constants of the sugar moiety in the molecule, and, in particular, the coupling constant of J˜9 Hz observed for the protons H—C(1″) and H—C(2″), and comparing these values with the H—C(1)/H—C(2) coupling constants reported for β-D-glucopyranosides and α-D-glucopyranosides, the D-glucopyranose moiety was proposed and the β-linkage undoubtedly identified.

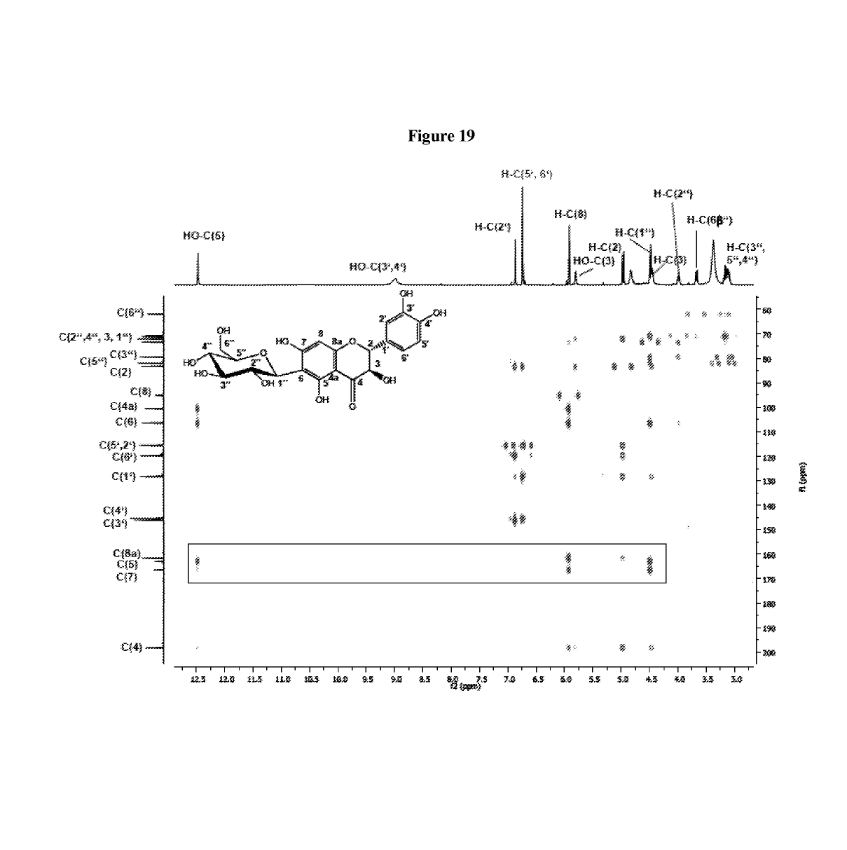

A comparison of the ¹³C NMR spectrum, in which 21 signals appeared, with the results of the heteronuclear single-quantum correlation spectroscopy (HSQC) experiment showing 12 signals, revealed 9 signals corresponding to quaternary carbon atoms. Unequivocal assignment of these quaternary carbon atoms and the hydrogen-substituted carbon atoms, respectively, could be successfully achieved by means of heteronuclear multiple bond correlation spectroscopy (HMBC) and HSQC. The typical ¹³C chemical shifts of the sugar part confirmed the D-glucopyranose. Additionally, the HMBC experiment revealed a correlation between the sugar proton H—C(1″) resonating at 4.49 ppm and neighboring carbon atoms C(5), C(7) and C(6), as well as no correlation to C(8a), thus demonstrating clearly the intramolecular 6-C-linkage of the b-D-glucopyranose to its aglycone (FIG. 19). The characteristic chemical shift of the carbon atom C(1″) at 73.0 ppm confirmed the C-linkage of the sugar part, and the taxifolin-6-C-b-D-glucopyranoside (25). The same strategy resulted in the structure of aromadendrin-6-C-β-D-glucopyranoside (26), isolated also from fraction M1 and detected as major constituent in fraction M2.

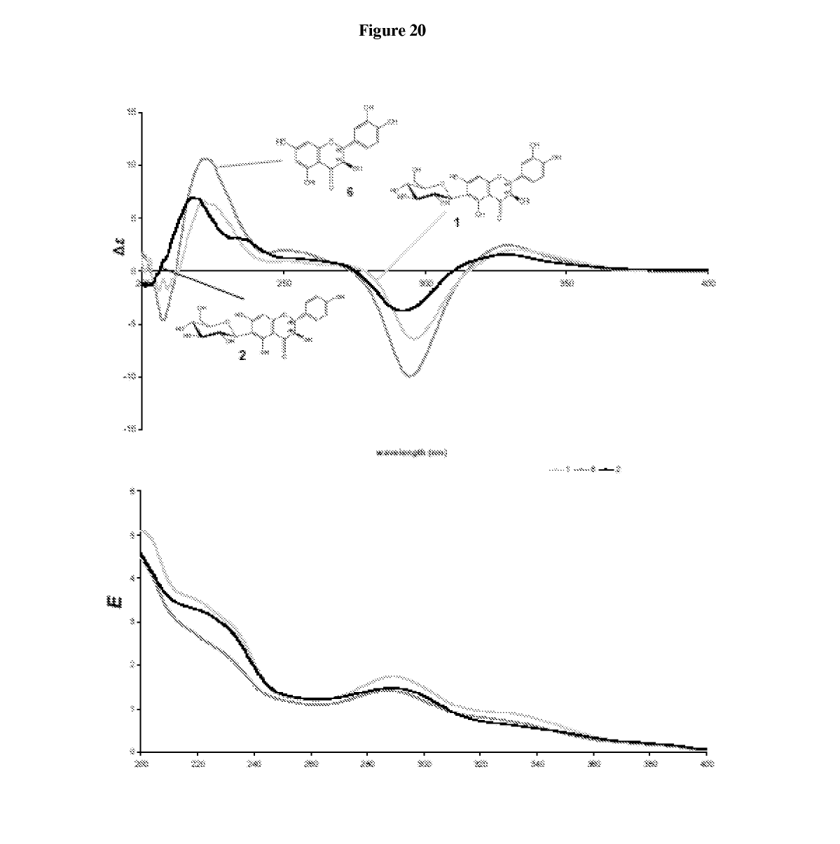

To clarify the configuration of the carbon atoms C(2) and C(3) present in the aglycone taxifolin and aromadendrin of compound 1 and 2, circular dichroism (CD) spectroscopic measurements were performed using the commercially available reference isomer (+)-(2R,3R)-taxifolin (6) as well as the isolated C-glycosides 1 and 2 (FIG. 20). The CD spectra of (+)-(2R,3R)-taxifolin (6) was well in line with literature data. The data obtained clearly demonstrated that the spectra of C-glycosides 1 and 2 isolated from fraction M1 were similar to the spectrum of (+)-(2R,3R)-taxifolin, therefore, the stereochemistry could be deduced as (2R,3R)-taxifolin-6-C-β-D-glucopyranoside (1) and (2R,3R)-aromadendrin-6-C-β-D-glucopyranoside (2).

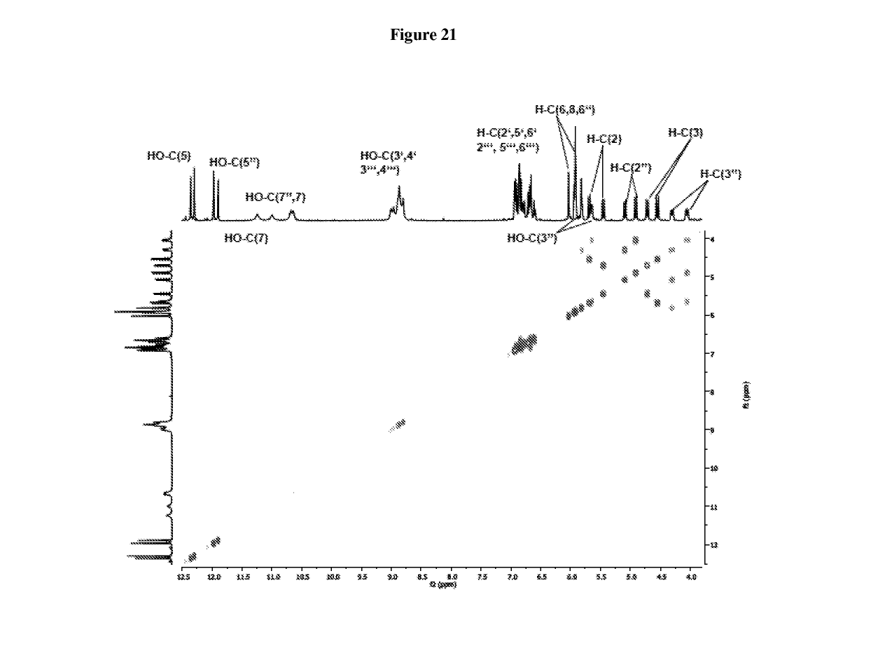

The antioxidative compounds 3-5 isolated from fractions M3-5 showed the typical absorption maxima expected for flavonoids, and high resolution LC-MS analysis confirmed the target compound to have the molecular formula C₃₀H₂₂O₁₃ for 3 and C₃₀H₂₂O₁₂ for 4 and 5, respectively. The ¹H NMR measurements of 3-5 in d₃-MeOD (RT), DMSO-d₆ (RT, 45° C.), acetone-d₆ (RT) as well as mixtures of acetone-d₆+DMSO-d₆ (9/1, v/v, RT, 8° C.) showed two series of signals typical for biflavonoids and duplication of nearly all signals, indicating the presence of two main rotational isomers (28-29). The sharpest signals were obtained at 8° C. (FIG. 21). The ¹H NMR spectrum of compound 3 showed four sharp exchangeable signals at 12.36 and 12.29 for HO—C(5) and 11.97 and 11.89 ppm for HO—C(5″), four broad exchangeable singlets from 10.64-11.24 ppm for the OH-groups of 7 and 7″, several broad exchangeable signals between 8.80-9.00 ppm for HO—C(3′,4′,3′″,4′″) and two exchangeable doublets for the protons HO—C(3″) at 5.64 and 5.80 ppm. Typical A-ring aromatic singlets for H—C(6,8,6″) from 5.81-5.94 ppm as well as characteristic C-ring aromatic protons resonating at 6.03-6.94 ppm could be observed. In addition, the two sets of aliphatic protons H—C(3,3″) and H—C(2,2″) coupling with each other were observed suggesting a GB-type biflavonoid (FIG. 21). The vicinal coupling constants of 11.3-12.1 Hz indicated their trans-diaxial relative configuration.

Unequivocal assignment of carbon atoms was successfully achieved by means of HMBC and HSQC. The HMBC experiment revealed a correlation between the proton H—C(3) resonating at 4.55 and 4.71 ppm and neighboring carbon atom C(8a″) and C(7″), as well as no correlation to C(5″), thus demonstrating clearly the intramolecular C-3/C-8″-linkage of the two flavanone monomers. Additionally, methylation of was performed, whereas after HPLC cleanup a mixture of two 8-fold methylated manniflavanone derivatives (3b,c) and nonamethylmanniflavone (3a) was obtained. The ¹³C chemical shifts of sterically hindered OMe substituents of flavonoids occur between 59-61 ppm as compared with 55-57 ppm for not ortho-disubstituted methoxyl groups. In 3a-c all aromatic OMe substituents were observed between 55.5-65.3 ppm. If the intramolecular linkage had been through C-3/C-6″ then C(5″)-OMe would have been sterically hindered and consequently deshielded. NMR data of compounds 3 and 3a are in line with partially described data for manniflavanone.

The 1/2D-NMR data of compound 4 was very similar to manniflavanone (3), besides the substitution pattern of the naringenin B-ring giving doublets (J=8.9) H—C(2′/6′) and H—C(3′/5′) at 7.21 and 6.73 ppm. Compound 4 was identified as GB-2, naringenin-C-3/C-8″ dihydroquercetin linked biflavanone.

In comparison to manniflavanone (3) 1/2D-NMR measurements of compound 5 revealed a methylene group at carbon atom C(3″) resonating at 43.1 ppm and therefore diastereotopic protons H—C(3ab″). Again, unequivocal assignment of carbon atoms could be successfully achieved by means of HMBC and HSQC and the linkage between the eridictyol monomers was confirmed via HMBC experiment, revealing a correlation between the proton H—C(3) resonating at 4.50 and 4.63 ppm and neighboring carbon atom C(8a″) and C(7″), as well as no correlation to C(5″), thus demonstrating clearly the intramolecular C-3/C-8″-linkage of the two eridictyol monomers. To the best of our knowledge, compound 5, which we have named buchananiflavanone, 3′,3′″,4′,4′″,5,5″,7,7″-octahydroxy-3,8″-biflavanone has never been described before.

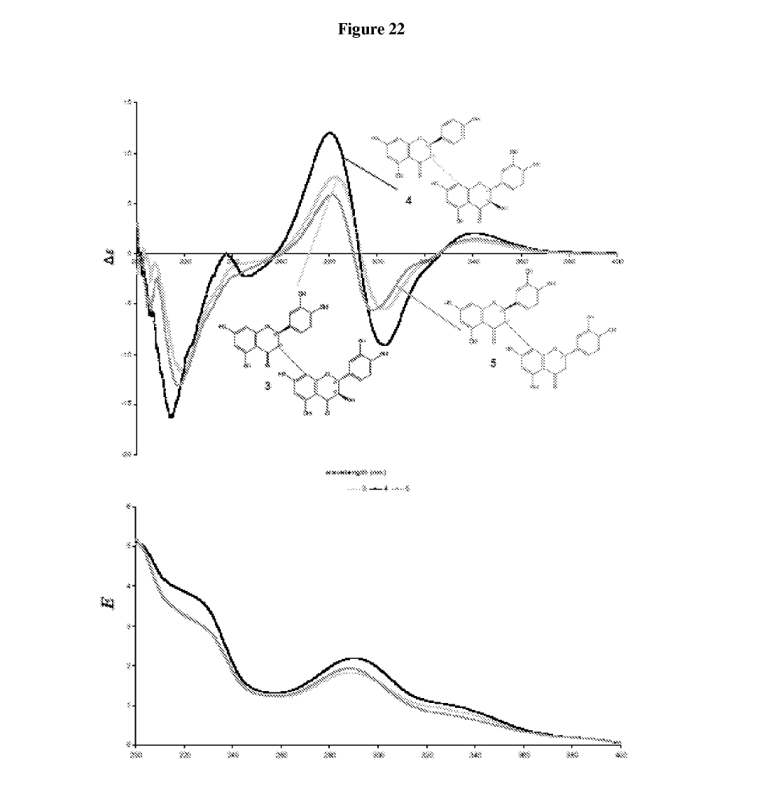

To clarify the configuration of the carbon atoms 1 C(2) and C(3) in compounds 3-5, the following CD spectroscopic measurements were performed with the commercially available (+)-(2R,3R)-taxifolin (6) as well as compounds 3-5. The CD spectra of GB-2 (4) (FIG. 22) was well in line with literature data. Consequently the stereochemistry of GB-2 (4) could be deduced as (2R,3S,2″R,3″R). Since the CD spectra of manniflavanone (3) was identical to GB-2 (4), the absolute configurations must be the same, and, thus the absolute configurations of manniflavanone must be revised to (2R,3S,2″R,3″R)-manniflavanone (3) (FIG. 22). The CD spectra of buchananiflavanone (5) (FIG. 22) was also very similar to (2R,3S,2″R,3″R)-GB-2 (4) and (2R,3S,2″R,3″R)-manniflavanone (3), showing typical CD bands with corresponding signs for 3-8″-biflavanones and/or mono flavanones like naringenin. In combination to Gaffield's rule and what has been shown by other investigations for n→p* and p→p* transitions of 3-8″-biflavanones consisting of either flavanones and hydroxyflavanones or flavanones and benzofuranones the stereochemistry of buchananiflavanone (5) could be deduced as (2R,3S,2″S).

### Example 6

### Antioxidative Activity of Isolated Compounds 1-5

Compounds 1-5 as well as known reference compounds with high antioxidative activity quercetin, rutin, (−)-epcatechin, ascorbic acid and (±)-naringenin were analyzed by means of ORAC and hydrogen peroxide scavenging assays (Table 6).

TABLE 6
Antioxidant activities of isolated compounds 1-5 and reference compounds.

| | H₂O₂ assay^{1,2} | | ORAC assay^{3,4} | | |
|---|---|---|---|---|---|
| | EC₅₀ (μM) | | (μmol TE/μmol) | | Literature^{5,6,7} |
| (2R,3R)-taxifolin-6-C-β-D-glucopyranoside (1) | 11.0 | (9.0-11.4) | 9.57 | (±0.50) | n.r.⁷ |
| (2R,3R)-aromadendrin-6-C-β-D-glucopyranoside (2) | 10.9 | (9.5-12.9) | 4.23 | (±0.08) | n.r.⁷ |
| (2R,3S,2″R,3″R)-manniflavanone (3) | 2.8 | (2.4-3.2) | 13.73 | (±0.43) | n.r.⁷ |
| (2R,3S,2″R,3″R)-GB-2 (4) | 2.2 | (1.9-2.6) | 12.10 | (±0.26) | n.r.⁷ |
| (2R,3S,2″S)-buchananiflavanone (5) | 14.4 | (12.8-16.5) | 10.50 | (±0.43) | n.r.⁷ |
| (+)-taxifolin (6) | 11.3 | (9.7-13.2) | 7.63 | (±0.68) | 9.74⁶ |
| ascorbic acid | 18.5 | (15.0-18.3) | 0.34 | (±0.10) | 0.95⁵ |
| rutin | 6.9 | (5.9-8.0) | 6.45 | (±0.28) | 6.01⁵; 13.70⁶ |
| quercetin | 6.1 | (5.3-7.1) | 5.61 | (±0.07) | 7.28⁵; 8.04⁶ |
| (−)-epicatechin | 4.1 | (3.7-4.6) | 9.65 | (±0.53) | 9.14⁶ |
| (±)-naringenin | 8.6 | (6.8-11.9) | 3.96 | (±0.19) | 9.23⁶ |

¹Each sample was analyzed by means of H₂O₂ assay by triplicate studies.
²Range in brackets represents 95% confidence interval.
³Each sample was analyzed by means of ORAC assay by quadruplicate studies.
⁴Numerical value in brackets represents SD.
⁵Values from Ou et al.;
⁶Values from Wolfe and Liu in which the stereochemistry of naringenin and taxifolin is not stated.
⁷n.r. not reported

In comparison to known very antioxidative single compounds, generally compounds 1-5 revealed relative high activity. By far the highest activity in both assays was observed for (2R,3S,2″R,3″R)-manniflavanone (3) and (2R,3S,2″R,3″R)-GB-2 (4), which showed outstanding activity in comparison to quercetin, rutin, (−)-epcatechin, ascorbic acid and (±)-naringenin as well as available literature data. Also, all EC₅₀ values of the hydrogen peroxide scavenging activity of compounds 1-5 are lower than in comparison to ascorbic acid. The newly isolated compound (2R,3S,2″S)-buchananiflavanone (5) revealed high activity in the hydrogen peroxide scavenging assay and also a very strong activity in the ORAC assay.

The application of the newly developed method for screening of natural antioxidative compounds by means of hydrogen peroxide scavenging assay dilution analysis in combination with ORAC assay on Garcinia buchananii extracts, revealed (2R,3R)-taxifolin-6-C-β-D-glucopyranoside (1), (2R,3R)-aromadendrin-6-C-β-D-glucopyranoside (2), (2R,3S,2″R,3″R)-manniflavanone (3), (2R,3S,2″R,3″R)-GB-2 (4) and the previously unreported compound (2R,3S,2″S)-buchananiflavanone (5) as highly antioxidative active constituents. When taken together, these findings indicate that G. buchananii bark extract is rich natural source of antioxidants with potential to be utilized as food supplements to fight chronic metabolic and degenerative diseases.

### Example 7

### Bioactivity of G. buchananii Fractions

Animals and Tissue Preparation for In Vitro Studies.

Adult male guinea pigs were used to obtain colons for motility assays and intracellular microelectrode recording in longitudinal smooth muscle-myenteric ganglia preparations to identify neuromechanisms. Animals were euthanized by exsanguination under deep isoflurane anesthesia. Intestines were collected in ice-cold Krebs solution (mM: 121 NaCl, 5.9 KCl, 2.5 CaCl₂, 1.2 MgCl₂, 25 NaHCO₃, 1.2 NaH₂PO₄ and 8 glucose; pH 7.4) and processed as needed for the specific experiments.

Motility Assays.

The guinea pig fecal pellet propulsive motility assay is a well-established model for analyzing neuroactive compounds that act on the enteric nervous system (ENS) to stimulate or inhibit gastrointestinal motility. Therefore, the motility assay was used as a high throughput-screening tool to identify bioactive MPLC fractions (M1-M8) that affect GI motility.

Segments of guinea pig distal colon (˜12 cm) were pinned in a 50 mL organ bath and continuously perfused with oxygenated Krebs solution at 36.5° C. Pellet propulsion velocities and contractile activities with spatio-temporal maps were measured by using the Gastrointestinal Motility Monitoring system and C-SOF-570 GIMM Software, respectively (GIMM; Med-Associates Inc., Saint Albans, Vt., USA) as described elsewhere. The test fractions were delivered to the colons in a Krebs bathing solution (serosal application) because G. buchananii extract and preparative thin layer chromatography (PTLC) fractions (PTLC1 and PTLC5) reduce motility with greater efficacy when applied serosally.

Findings.

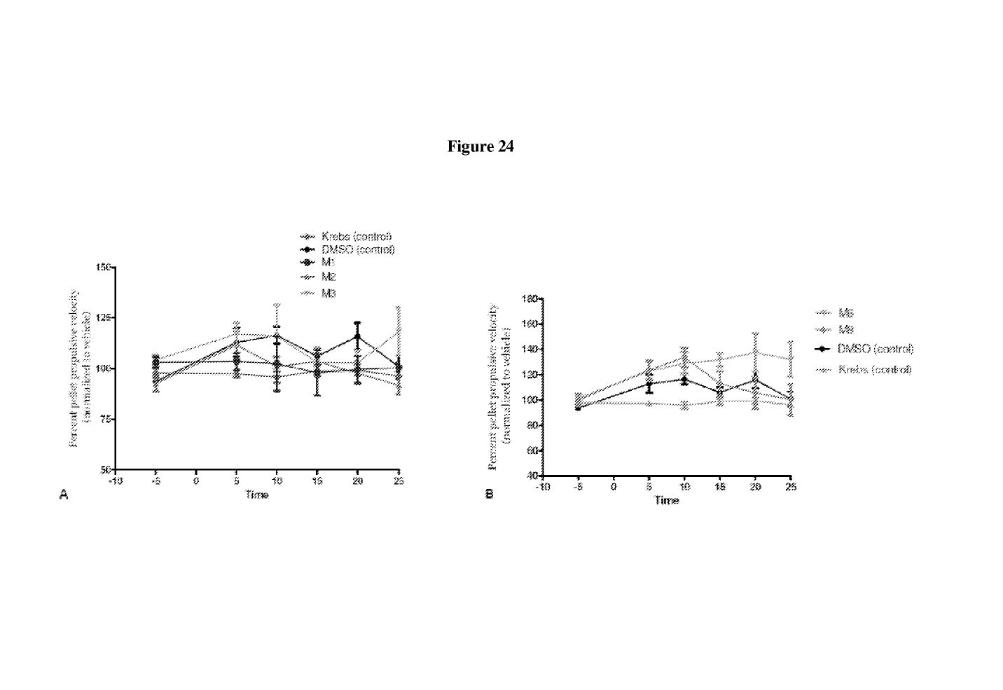

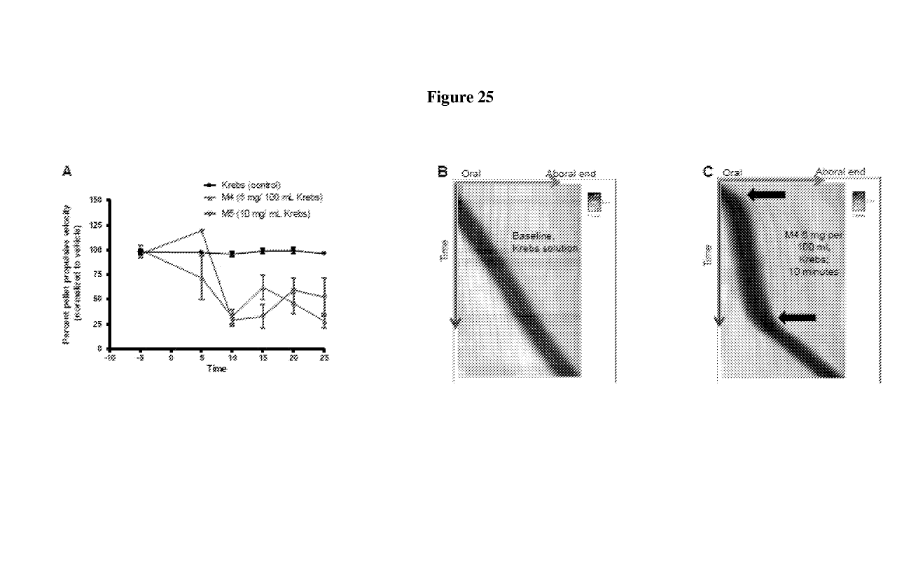

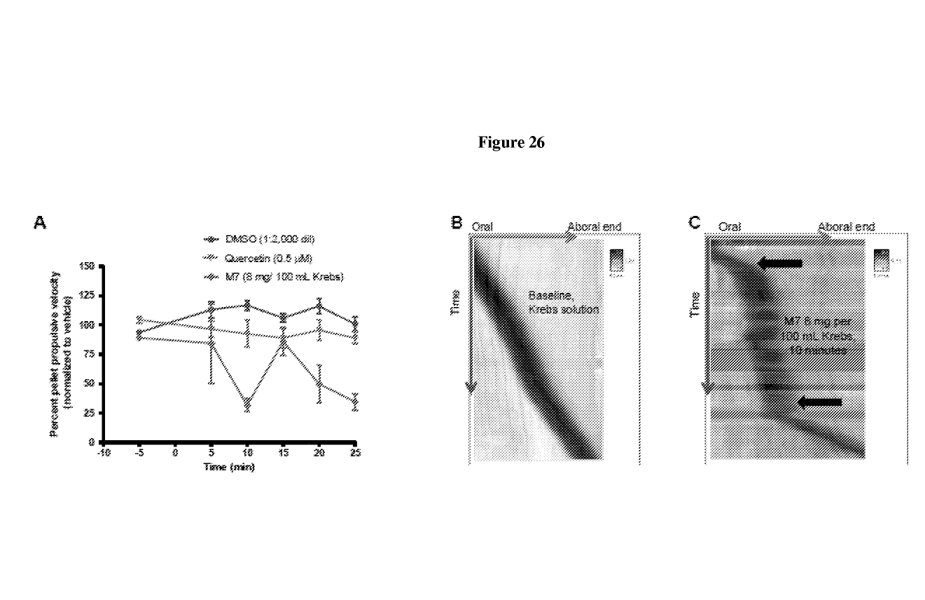

The results of motility assays in isolated guinea-pig distal colons showed that medium pressure liquid chromatography (MPLC) fractions: M1-M3 did not affect pellet propulsion velocity (FIG. 24A), suggesting that these fractions do not contain neuroactive anti-motility compounds. M6 and M8 increased colon motility, suggesting they have neuroactive, prokinetic compounds (FIG. 24B). M4, M5, and M7 inhibited propulsion (FIGS. 25-26) suggesting these fractions of G. buchananii stem bark extract contain neuroactive, antimotility compounds.

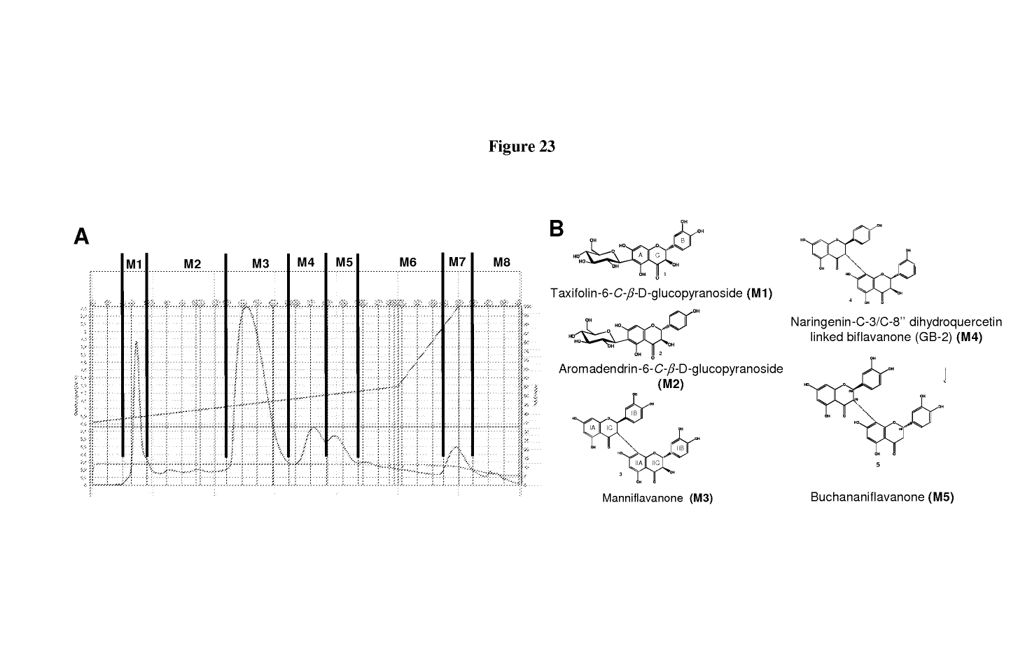

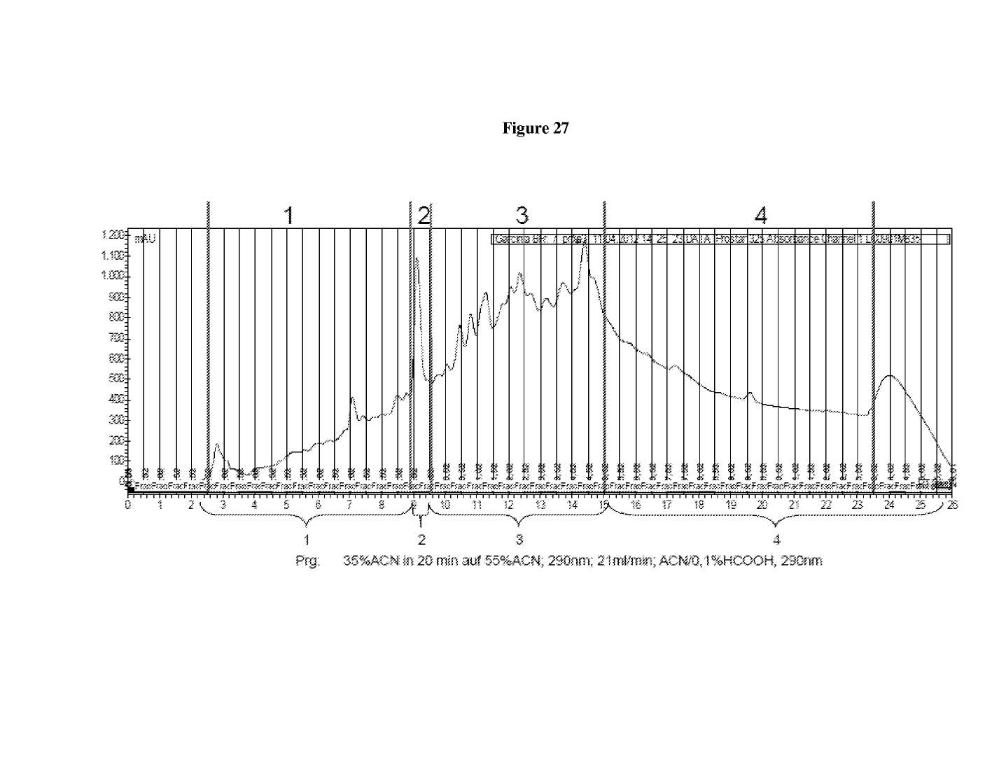

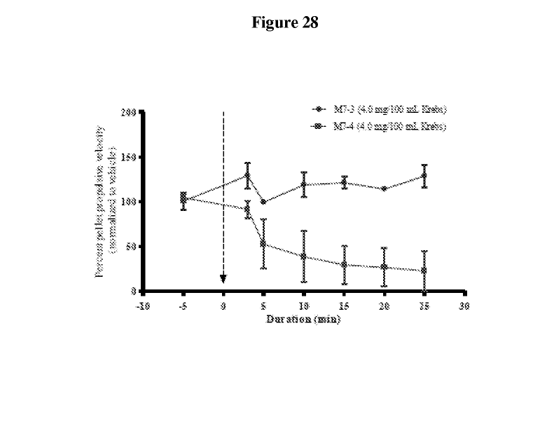

In the effects to identify bioactive compounds, we have isolated and identified the main compounds in fractions M1-M5 (Stark et al., 2012) and found that the main compound found in M4 is naringenin-dihydroquercetin-linked biflavanone. M5 contains a novel flavanone, buchananiflavanone (FIG. 23A-B). To identify the bioactive compound in M7, the fraction was subjected to a deeper analysis by using bioactivity-guided sub-fractionation. Medium pressure liquid chromatography was used to separate M7 into four subfractions (FIG. 27). The sub-fractions (M7-1-4) were analyzed using motility assays to identify the sub-fraction having anti-motility effects for ultimate isolation and characterization of the active compound. We found that when used at the same concentration as M7 whole fraction, only one sub-fraction of M7, which we have termed M7-4, inhibited pellet propulsion (FIG. 28).

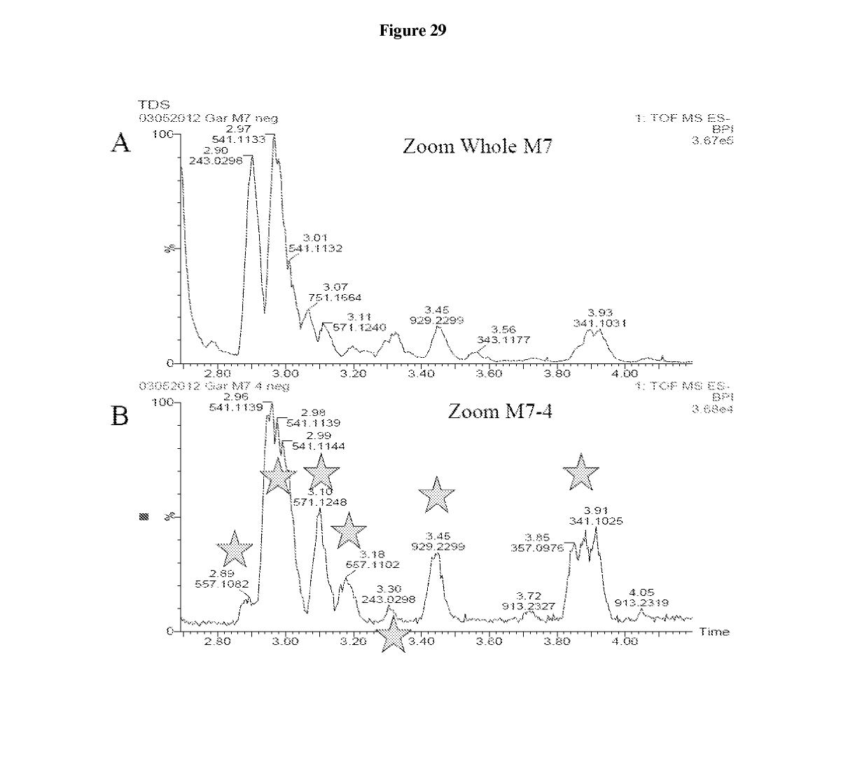

The isolation and characterization of the compound in M7-4 sub-fraction is being undertaken. Preliminary findings suggest that the active compound is likely a xanthone or flavanoid or flavonoid or other unknown compounds (FIG. 29A-B). To summarize, two compounds—naringenin-C-3/C-8″dihydroquercetin linked biflavanone (GB-2) identified by Jackson, B.; 1971 and buchananiflavanone, a novel biflavone from G. buchananii that we identified for the first time inhibits guinea pig colon propulsive motility, which is consistent with the findings obtained by using the extract and the PTLC1 and PTLC5 fractions, collected using silica gel fractionation. Together our findings support the notion that G. buchananii stem bark extract could be the basis of new therapies against diarrheal diseases including novel anti-motility compounds.

### Example 8

### Garcinia buchananii and its Derivative Antimotility Flavanones Inhibit Neurotransmission

M4 (GB-2) and MS (Buchananiflavanone) Inhibit Neurotransmission in Mechanosensory Neurons

Voltage sensitive dye imaging (Mazzuoli G. and colleagues 2009) was performed on whole mount preparations from guinea pig ileum myenteric plexus. The staining was performed with the dye Di-8-ANEPPS (2.2 μl in 1500 μl Krebs solution). In all the experiments 1 μM nifedipine was used to prevent muscle contraction. The experiment was performed with brief (500 ms) applications of Garcinia extract (0.5 g/100 ml Krebs) directly onto the ganglia. 18±7% of the myenteric neurons responded to this acute application with a spike frequency of 0.8 Hz. The first spike appeared with a delay of 465±233 ms after the stimulus application.

Next the ganglia were mechanically probed (control) with an intraganglionic injection method; then Garcinia extract (0.5 g/100 ml Krebs) was superfused to the entire tissue and another mechanical stimulation was performed. A third mechanical stimulation was done after 30 min wash out.

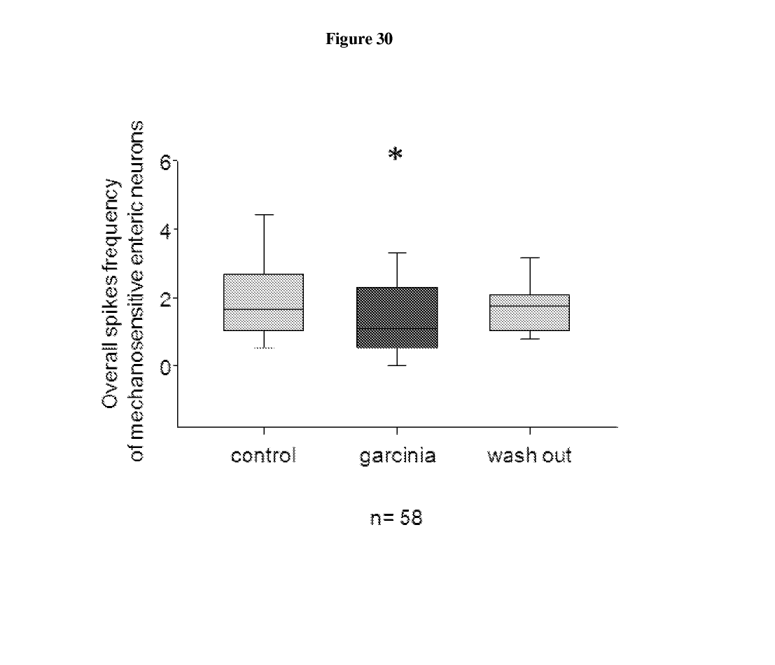

Results showed a significant decrease in the mechanosensitivity response of the myenteric neurons after perfusion with Garcinia that was recovered after wash out (FIG. 30).

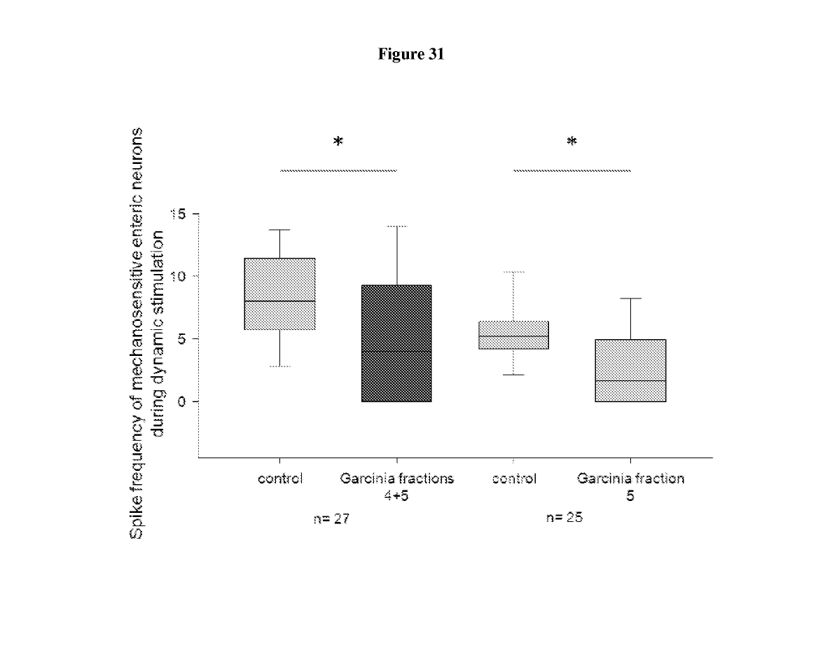

Experiments were also performed examining mechanical stimulation and perfusion of a mixture of G. buchananii MLPC fractions M4 and M5 or the M5 alone. M4 was used 6 mg in 100 ml HEPES modified Krebs solution. M5 was used 10 mg in 100 ml. M5 was also tested at lower concentration but had no significant effect. Results indicated that mechanosensitive responses of the myenteric neurons are significantly decreased after perfusion of M4 and M5 as well as with M5 alone (FIG. 31).

To summarize, these first results indicate that G. buchananii per se and in particular its fractions M4 and M5 have a direct effect on mechanosensitive enteric neurons of the guinea pig ileum myenteric plexus. These findings strongly suggest that M4 and M5 have compounds that inhibit neurotransmission, which are GB-2 and buchananiflavanone, respectively.

### Example 9

M4 (GB-2), M5 (Buchananiflavanone) and M7 Inhibit Neurotransmission: Evidence Based on Inhibition of Junction Potentials.

A standard intracellular microelectrode-recording assay was used to identify the G. buchananii stem bark extract MLPC fractions containing compounds that inhibit transmission in the myenteric ganglia of guinea pig distal colon. In order to conduct a rapid screening assay of all the fractions, the effect of M4-M5 and M7 on evoked inhibitory and excitatory junction potentials in the inner circular muscle in intact muscularis externa in the guinea pig distal colon were analyzed. Junction potentials in the GI tract are electrical events in smooth muscle cells elicited by neurotransmitter release. Excitatory neurotransmitters such as acetylcholine and substance P elicit excitatory junction potentials (EJPs) and a contraction oral to the contracting segment, which may or may not contain a food bolus or a pellet. Conversely, release of inhibitory neurotransmitters such as nitric oxide, and purines elicits inhibitory junction potentials (IJPs) and a relaxation. Typically, IJPs occur aboral to the contracting segment. Technically, in intracellular recording, studying junction potentials in smooth muscle cells are “readout” neurotransmission is relatively easy and quicker than direct recording from neurons. Therefore, this approach was used as a quick screening method to identify MLPC fractions containing neuroactive compounds.

Junction potentials were evoked by transmural stimulation (single pulses of 50-80 volts, 250 ms duration) on a grass S88 stimulator. Junction potentials were recorded from impaled circular smooth muscle cells by using published procedures. A segment of the guinea pig distal colon was cut, pinned and stretched mucosal side up in a ˜2.5 ml recording chamber. The mucosal and submucosal layers were dissected away with sharp forceps to expose the circular muscle layer. Tissues were maintained at 36.0-36.5° C. by continuous perfusion with an oxygenated Krebs solution (˜10 mL/min) containing nifedipine (2 μM) to inhibit smooth muscle contraction. Tissues were observed using an inverted Nikon Ti-S microscope (Nikon Instruments, Inc., Melville, N.Y., USA). Glass microelectrodes were loaded with 0.5 M KCl in the tip and then backfilled with 2.0M KCl, with resistances ranging from 100 to 120 mega Ohms was used to transmembrane potential was measured with MLB870B71 intracellular recording system amplifier (ADInstruments, Colorado Springs, Colo., USA) and signals were acquired and analysed with PowerLab 8/30 with LabChart Pro fo (ADInstruments, Colorado Springs, Colo., USA).

Findings:

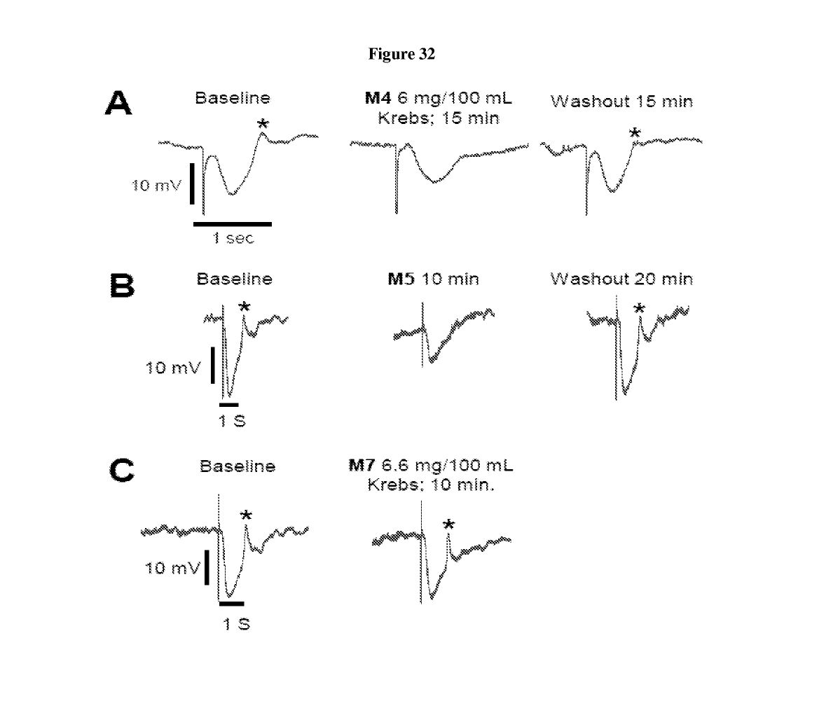

This bioactivity analysis showed that M4 and M5 reduced the amplitudes of the excitatory and inhibitory junctions potentials. Noteworthy, a washout rapidly restored junction potentials. Although M7 and its derivative, M7-4 inhibit colon motility, M7 did not affect neither the excitatory nor the inhibitory junction potentials, which is contrary to our expectations (FIG. 32, n=2). Unlike M4 and M5, M7 did not inhibit excitatory junction potentials (asterisks).

By using both the voltage sensitive dye imaging and intracellular microelectrode recording it was found that GB-2 (M4), and buchananiflavanone (M5) inhibit neurotransmission as evidenced by inhibition of mechanosensory neurons in the myenteric ganglia in the guinea pig ileum, and junction potentials in the circular smooth muscle layer of guinea pig distal colon. When taken together with motility data, these findings correspond with the observations made using the crude extract and confirm that GB-2 (M4) and buchananiflavanone (M4) inhibit neurotransmission presumably by inhibiting release of neurotransmitters or other mechanisms.

Contrary to our expectation, M7 (4 mg/100 mL Krebs) did not affect junction potentials. Two lines of evidence support the notion that M7 has neuroactive anti-motility compounds, which inhibit neurotransmission. First, as a whole, M7 inhibited pellet propulsion in the guinea pig distal colon. Second, after separating M7 into sub-fractions, sub-fraction M7-4 inhibited pellet propulsion in the guinea pig distal colon. Collectively, these observations indicate the need of a detailed analysis including measuring the dose response curves of M7-4 and analyze the anti-motility effects to identify sub-fractions of M7-4 in order to isolate, test and characterize the neuroactive compounds in M7-4.

We have established that inhibition of fEPSP by G. buchananii stem bark extract in the myenteric ganglia of guinea pig distal colon does not involve activation of α2-adrenoceptors and opioid receptors ([3]). The findings summarized here strongly support these original observations and show that the bioactive compounds are biflavanones, in particular, GB-2 in M4, buchananiflavanone in M5 and flavanones, xanthones or unknown compounds in M7 that inhibit neurotransmission.

The peristaltic reflexes that underlie intestinal motility depend on the release of 5-HT from mucosal sensory transducer cells called enterochromaffin (EC) cells and subsequent activation of 5-HT receptors on intramucosal sensory nerve endings as well as through intrinsic rhythmic electrical activity in the gut wall. Research has shown flavonoids and xanthones extracted from various parts of plant from the genus Garcinia affect 5-HT receptors. For example, gamma-mangostin, the xanthone from a fruit hull of G. mangostana, inhibits 5-HT2A receptors in the CNS, vascular smooth muscles and blood platelets. Quercetin, the flavonoid found in Garcinia plants, inhibited mouse and frog, Xenopus laevis oocyte 5-HT3A oocytes receptors. Likewise, scutellarin and ikonnikoside, the flavonoids from Scutellaria lateriflora, affect 5-HT7 receptors, while Kadowaki, M. and co-workers (1997) demonstrated that concurrent blockage of 5-HT3- and 5-HT4-receptors drastically inhibited colonic propulsive motility and markedly reduced diarrhea in conscious rats and mice. We have recently shown that G. buchananii bark extract and its fractions (PTLC1 and PTLC5) reduce colon motility by affecting 5-HT3 and 5-HT4 receptors. Although the mechanisms of these compounds are not known, our studies employing the crude extract and PTLC fractions suggest that it is possible that these compounds act at least in part by inhibiting 5-hydroxytryptamine (5-HT) receptor subtypes 3 and 4. Still it is possible that neuroactive compounds in G. buchananii bark extract inhibiting purinergic receptors, 5-HT1A receptors, and presynaptic neurotransmitter release. These assumptions are supported by the finding that flavonoids such as quercetin can inhibit acetylcholine release or interfere with presynaptic G-protein coupling and neurotransmitter release. It is possible that neuroactive compounds in G. buchananii extract inhibit presynaptic receptors on cholinergic neurons. In addition, plant polyphenols and the flavonoid quercetin are believed to selectively block nicotinic acetylcholine receptors. This suggests that this is another potential mechanism that could be utilized by G. buchananii extract-derivative flavanones to inhibit fEPSPs.

Presynaptic receptors are coupled to G-proteins, and some compounds can act via these receptors to inhibit neurotransmitter release via activation of K⁺ channels causing either (a) a reduction of action potential efficacy, (b) inhibition of Ca²⁺ channels that leads to impairment of exocytotic machinery or (c) inhibiting neurotransmitter release from storage vesicles. Presynaptic inhibition of neurotransmitter release in the ENS depends on pertussis toxin (PTX)-sensitive presynaptic receptor-G-protein coupling mechanisms. All these a possible candidate targets for GB-2, buchananiflavanone and M7-4 that need to be tested.

Their actions are readly reversible, which corresponds with the observations made previously when using the whole G. buchananii stem bark extract.

### Example 10

### Garcinia Stem Bark Extract Contains Bioactive Compounds that Affect Sensory Neurotransmission

The activation of sensory neurons by signal molecules is critical for initiating propulsive motility, secretion and the transmission of information to the central nervous system. Therefore, it is possible that G. buchananii extract or GB-2 or buchananiflavanone or M7-D or all three reduce motility by inhibiting the sensory neurons in the gut.

In electrophysiology the ENS neurons presenting a shoulder on the repolarizing phase of the action potential and a large after-hyperpolarizing potential (AHP) at the soma are classified as intrinsic sensory neurons. These neurons are also called the intrinsic primary afferent neurons (IPANs) or AH/type 2 neurons. Those without these features are generally called S-type neurons. Another class of sensory neurons is the non-spiking neurons, which do not generate action potentials in response to depolarizing current pulses.

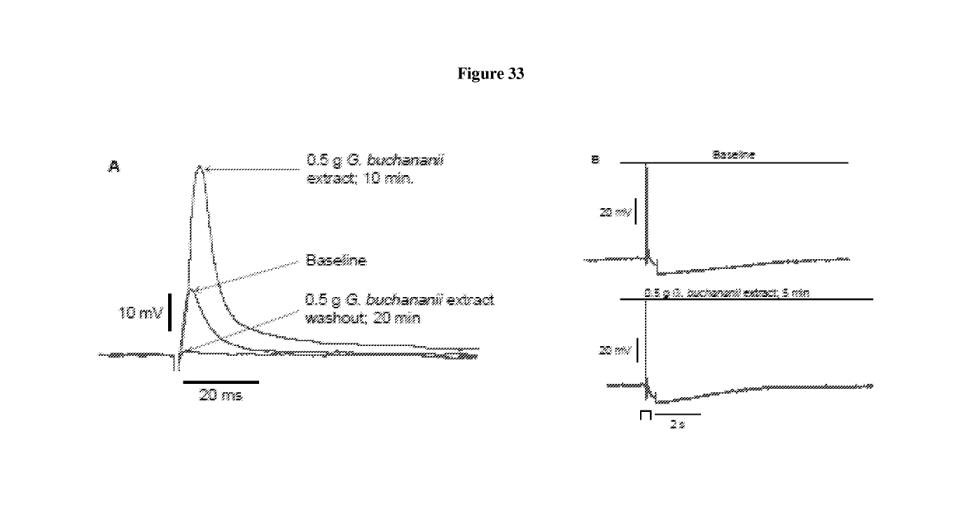

In our preliminary studies in the myenteric ganglia of guinea pig ileum, we observed that G. buchananii extract did not affect the resting membrane potential of AH neurons but caused increased firing of antidromic action potentials (FIG. 33A). In some cases the extract did not affect the firing properties of AH neurons (FIG. 33B), while in other cases, G. buchananii extract elicited an increased rate of spontaneous firing activity. The ileum was chosen because it was suitable for these studies because the population of AH neurons is higher in ileum compared to colon (30% versus 5-10% of all myenteric neurons). The excitatory post-synaptic potentials (fEPSPs), which are common in S-type neurons are rarely seen in AH neurons. Preliminary data suggested that fEPSPs to AH neurons are reduced by G. buchananii bark extract, which is similar to previous findings of synaptic inhibition in S-type neurons. It is possible that GB-2, buchananiflavanone and M7-D inhibit motility by affecting the excitability of AH-type neurons, which is another avenue that should be pursued rigorously.

### Example 11

### Garcinia buchananii stem bark extract Derivative Compounds and Recombinants of these Compounds in their Natural Concentrations, have Extraordinary Antioxidant Power

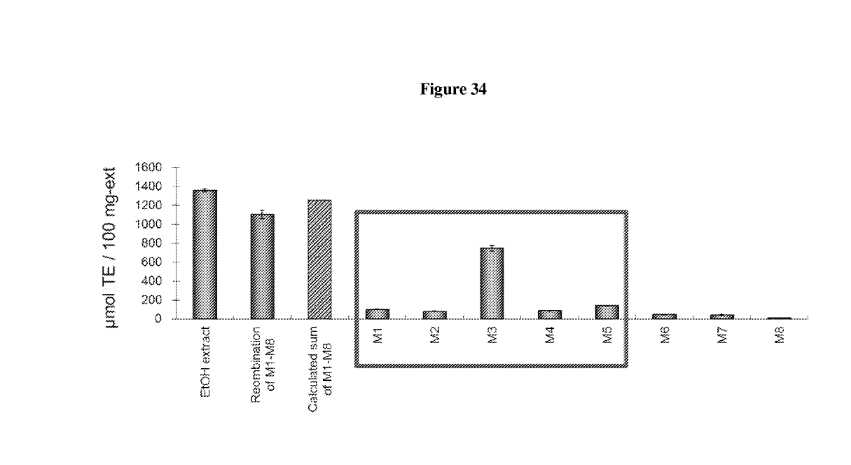

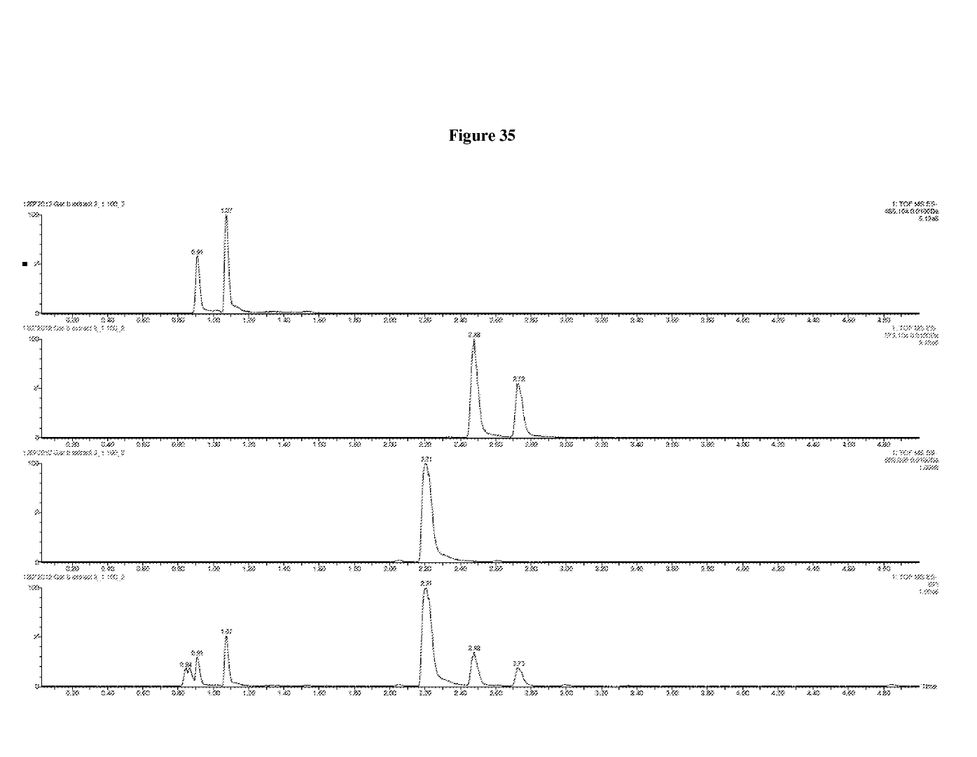

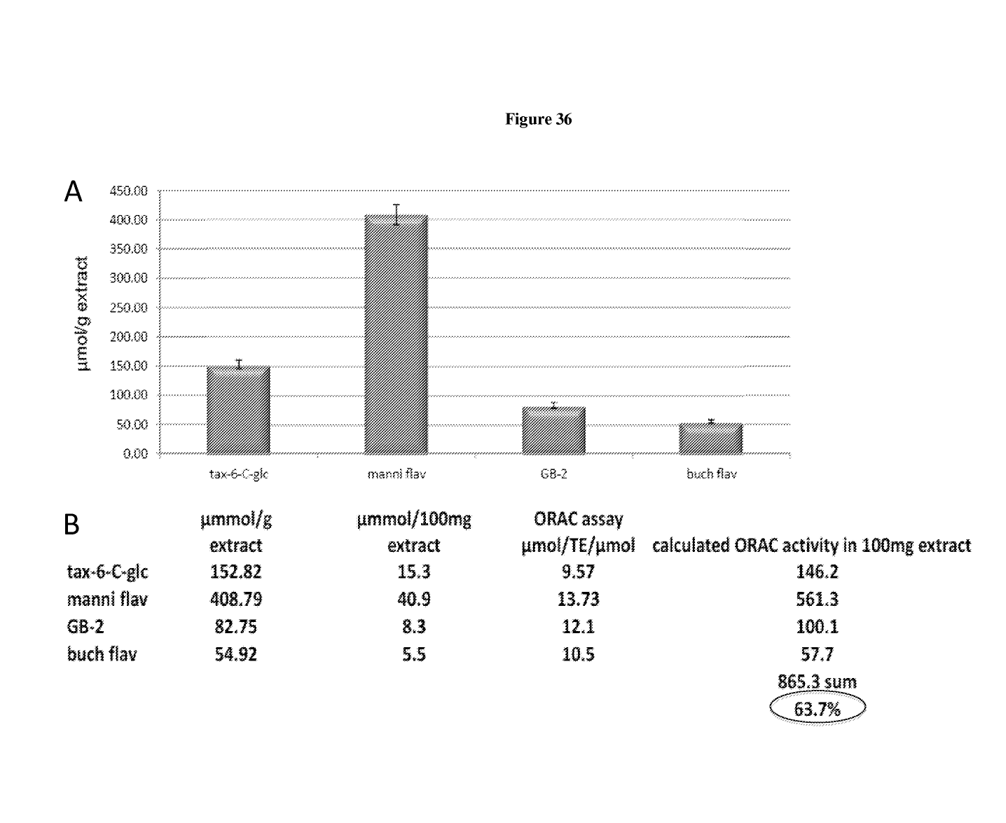

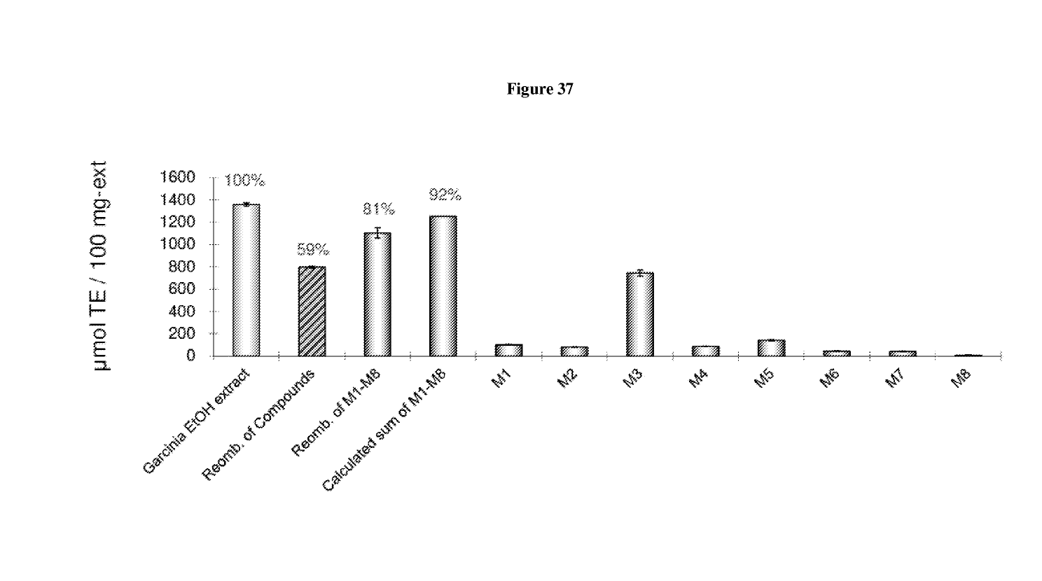

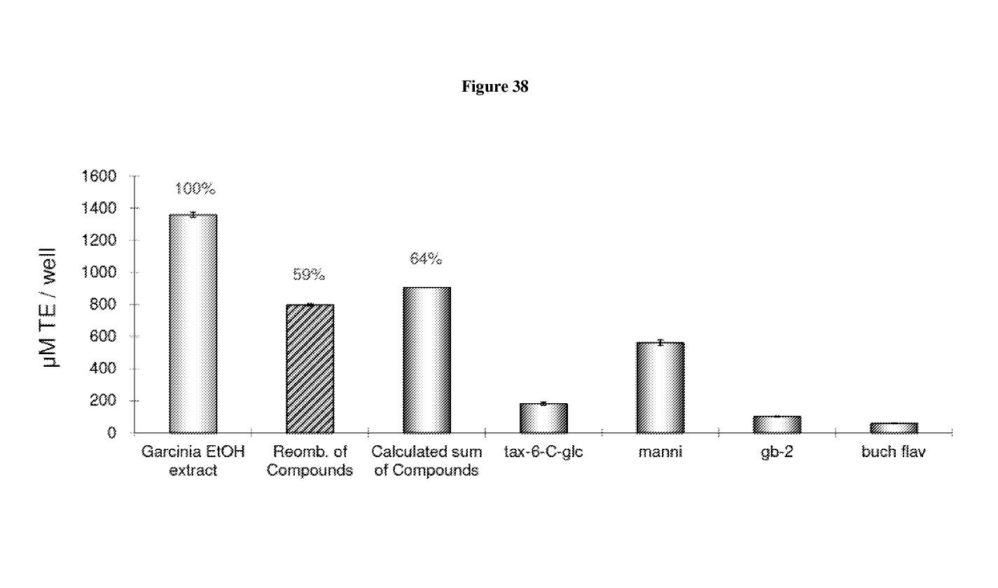

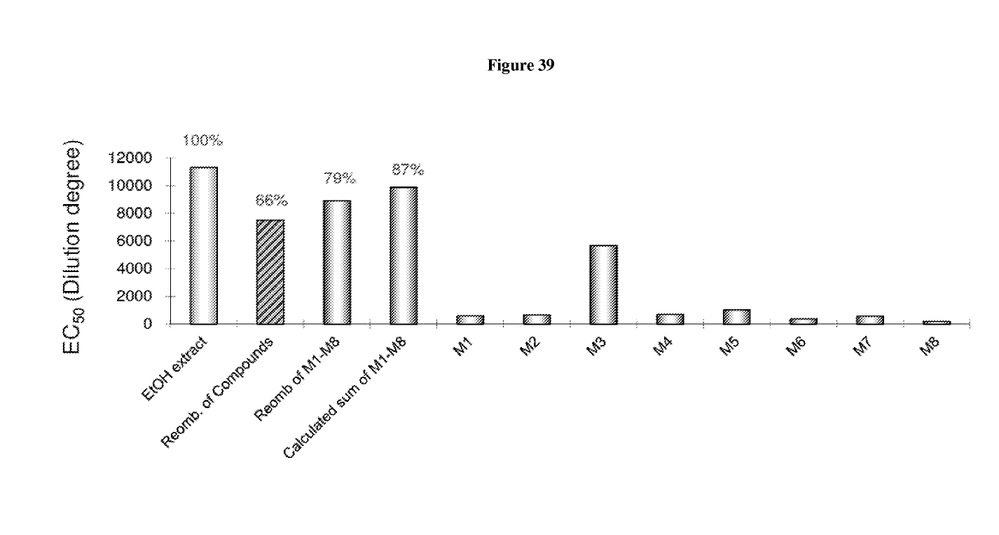

Whole G. buchananii stem bark extract and its fractions (M1-8) tested in the natural concentration using the ORAC assay. G. buchananii stem bark extract and the M1 and M3-M5 fractions collected by using medium pressure liquid chromatography (MPLC) have powerful antioxidative activity (FIG. 34). The four main antioxidative compounds were quantified using UPLC-time of flight mass spectroscopy as shown in FIG. 35. Quantitative demonstration of the antioxidative activity of the four compounds isolated from G. buchananii stem bark extract was found using the ORAC assay (FIGS. 6 and 36). FIG. 39 shows the same data using the H₂O₂ assay. The antioxidative activity of whole aq. EtOH extract was 1359 μmol TE. The calculated ORAC activity of the four compounds with powerful anti-oxidative activities combined is 64% of the whole extracts (sum of single activity). FIG. 37 shows that the four compounds isolated from M1 and M3-M5 have 59% of the antioxidative activity as measured by ORAC. FIG. 38 shows the antioxidant activity of each of the compounds in isolation.

### Example 12

### Garcinia buchananii Stem Bark Extract Derivative Compounds Inhibit Ca²⁺ Signaling in Smooth Muscle Cells

In studying the anti-diarrheal effects of G. buchananii stem bark extract in rats, it was observed that the extract causes smooth muscle relaxation. Consequently, the effect of G. buchananii stem bark extract on calcium transients was investigated and the compound with relaxant actions was identified by testing the extract's fractions and purified compounds on action potentials in gallbladder smooth muscle (GBSM) and slow wave action potentials in circular smooth muscle cells (CSMC) in the guinea pig and porcine colons.

Materials and Methods:

Calcium imaging and standard intracellular microelectrode recording were used to determine the effect of G. buchananii extract on the discharge and frequency of Ca²⁺ transients, Ca²⁺ flashes and Ca²⁺ waves. Six fractions (M1-M5 and M7) collected using medium pressure liquid chromatography and manniflavanone, a pure compound from M3 were analyzed for the effect on the resting membrane potential and action potentials in intact GBSM preparations, and slow wave action potentials in CSMC in guinea pig and porcine colon.

Results:

Garcinia buchananii stem bark extract (0.5 g bark powder/100 mL Krebs) inhibited Ca²⁺ flashes and Ca²⁺ waves, and spontaneous action potentials, in guinea pig GBSM. FIGS. 40A and B demonstrate that G. buchananii extract contains compounds that inhibit Ca²⁺ transients (Ca²⁺ flashes and Ca²⁺ waves) in smooth muscle cells. Panel A shows pictures of guinea pig gallbladder smooth muscle fascicles, and traces of Ca⁺ flashes (synchronized peaks with asterisks) and Ca²⁺ waves (asynchronous, unlabeled peaks) generated from selected smooth muscle cells (blue and red blocks in the pictures). Panel B shows that compared with control (panel A) G. buchananii extract (0.5 g stem bark powder/100 ml physiological saline solution; PSS) causes rapid inhibition of Ca⁺ flashes and Ca⁺ waves in gallbladder smooth muscle cells. G. buchananii stem bark extract reduced the discharge of Ca²⁺ flashes guinea pigs smooth muscle when applied on mucosal surface (full thickness gallbladder preparation; FIG. 40C) and directly on intact muscle preparation from gallbladder (muscularis; FIG. 40C) and distal colon (muscularis; FIG. 40D).

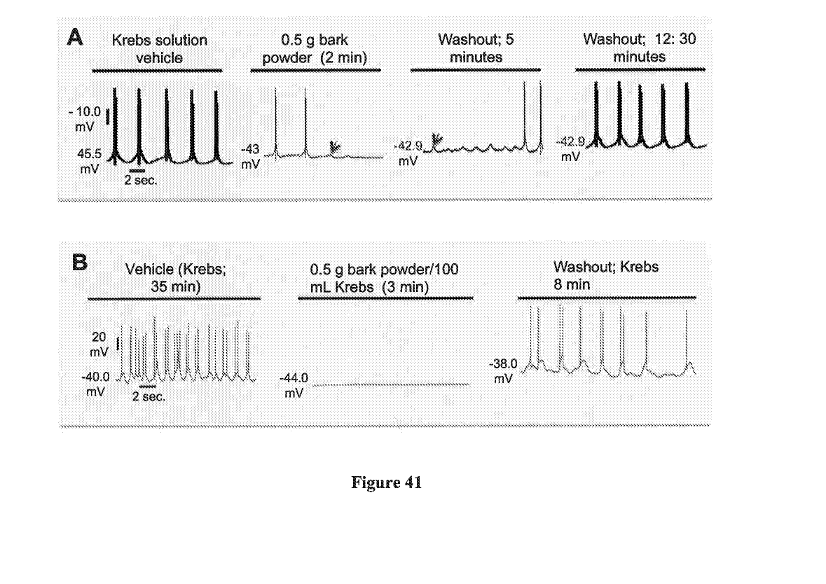

G. buchananii extract contains compounds that inhibit action potentials in smooth muscle cells. FIG. 41A shows traces of action potentials (AP) from guinea pig gallbladder smooth muscle cells (GBSM; standard intracellular microelectrode recording in intact tissues) showing that compared with Krebs solution vehicle, the extract (0.5 g stem bark powder/100 ml vehicle) inhibits spikes of AP (rapid membrane depolarizations due to Ca²⁺ influx via L-type calcium channels and sub-threshold membrane depolarizations (arrows). These observations suggest tat the extract has compounds or a compound that inhibits L-type Ca²⁺ channels and intracellular Ca²⁺ handling. Washout restores sub-threshold membrane depolarizations first (arrows, 2 & 5 min washout) followed by superimposed spikes (C). Overall, washout restores AP discharge to the original rhythmic pattern and frequency.

Demonstration of the effect of whole G. buchananii extract on slow wave action potentials (SW) and membrane potentials in guinea pig colon smooth muscle cells is shown in FIG. 41B. FIG. 41B shows traces of slow wave action potentials (SW) showing that compared with Krebs vehicle, G. buchananii extract from 0.5 g stem bark powder/100 ml Krebs inhibits SW in circular smooth muscle cells in intact preparations of muscularis externa. Washout rapidly restores SW. The rhythmicity and synchronicity of SW returns to normal after a washout.

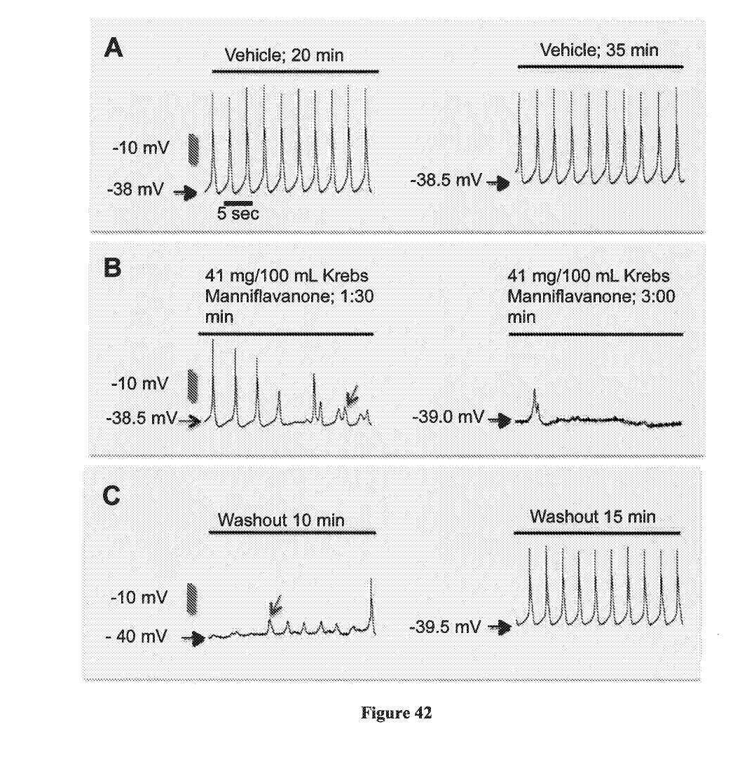

FIG. 42 shows traces of action potentials from gallbladder smooth muscle cells showing that compared with Krebs vehicle (A), manniflavanone (B; 41.0 mg/100 mL Krebs) inhibits action potentials (AP). Like the whole extract, manniflavanone inhibited spikes of AP (rapid, Ca²⁺ influx-dependent membrane depolarizations) first, suggesting the extract and manniflavanone inhibit L-type Ca²⁺ channels before inhibiting the subthrehold membrane depolarizations (arrow; B). Washout restored subthrehold membrane depolarizations first (arrow; C), followed by superimposed spikes. Compare with this Figure with FIG. 42A.

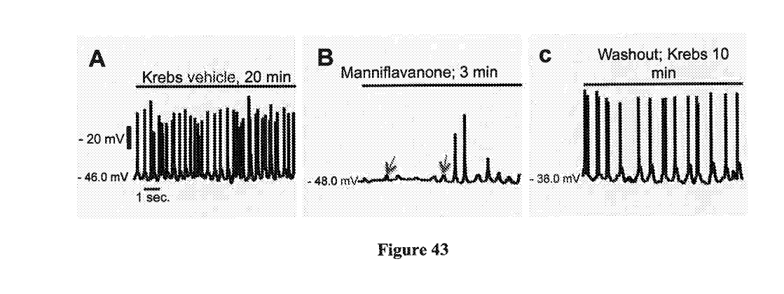

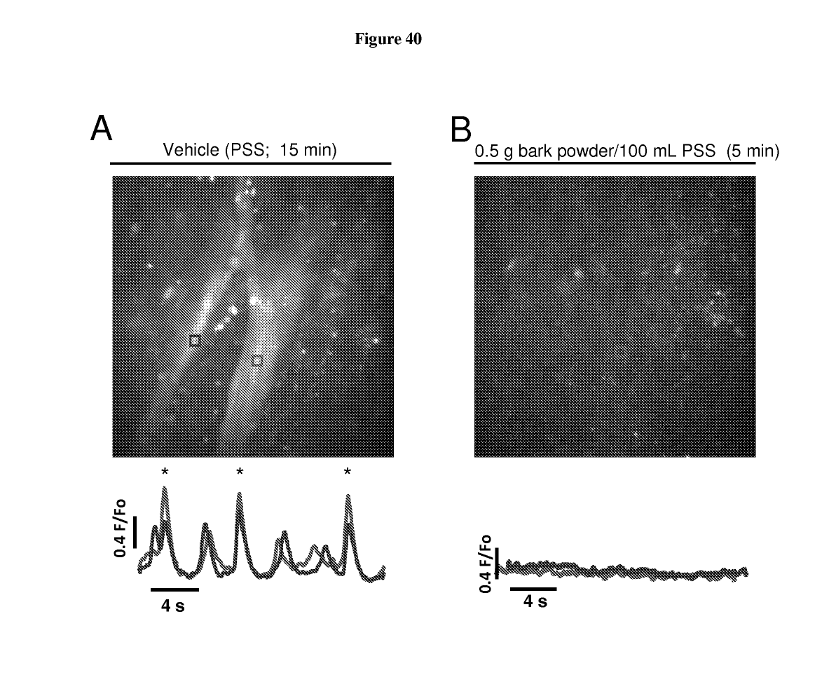

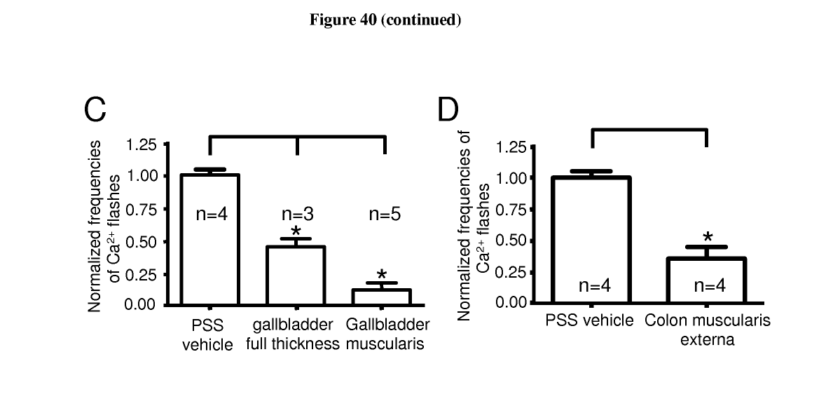

FIG. 43 shows traces of slow wave action potentials (SW) obtained by intracellular recording from guinea pig colon smooth muscle cells in the inner circular layer of muscularis externa. Compared with Krebs vehicle (A) manniflavanone (41.0 mg/100 mL Krebs) inhibits spikes of SW (rapid Ca²⁺ influx-dependent membrane depolarizations) and subthrehold membrane depolarizations (arrows) suggesting the extract inhibits L-type Ca²⁺ channels (B) in intestinal smooth muscle cells. Washout restores these events to normal frequency and similar rhythmic pattern. Compare with this Figure with FIG. 42B.

The extract, its MLPC fraction, M3 and manniflavanone, a compound isolated from M3 inhibited action potentials in GBSM and slow waves action potentials in CSMC. The effects of the extract, M3 and manniflavanone were readily reversed by a washout.

Conclusions:

Garcinia buchananii extract and its derivative manniflavanone, inhibit L-type calcium channels and intracellular Ca²⁺ mobilization and have smooth muscle relaxation effects. These findings suggest that Garcinia buchananii extract and manniflavanone have potential as new drugs in therapies requiring the use of antioxidants, and Ca²⁺ channel blockers for lowering blood pressure in patients with hypertension, abnormally rapid heart rhythms and angina, muscle spasms, prevention of migraine headaches and epilepsy.

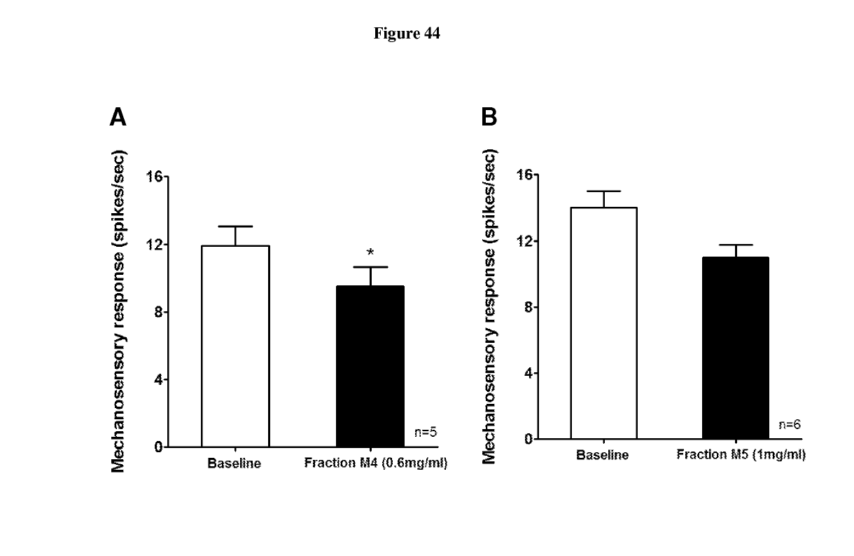

Data presented in FIG. 44 show that M4 and M5, the MPLC fractions of Garcinia buchananii stem bark extract contain anti-nociceptive compounds

FIG. 44 shows colonic afferent recordings with G. buchananii stem bark extract fractions M4 (A) and M5 (B). Both M4 and M5 inhibited nociceptor function, but not to the same extent as the whole G. buchananii stem bark extract (shown previously). These findings suggest that other fractions we have demonstrated to posses neuroactive compounds M7 and M6 might be involved in nociceptor inhibition. Note: The main bioactive compounds in M4 and M5 are GB-2 and buchananiflavanone, respectively.

### Example 13

### Matching PTLC1 and PTLC5 with High Pressure (HPLC) and Medium Pressure (MPLC) Liquid Chromatographic Separations

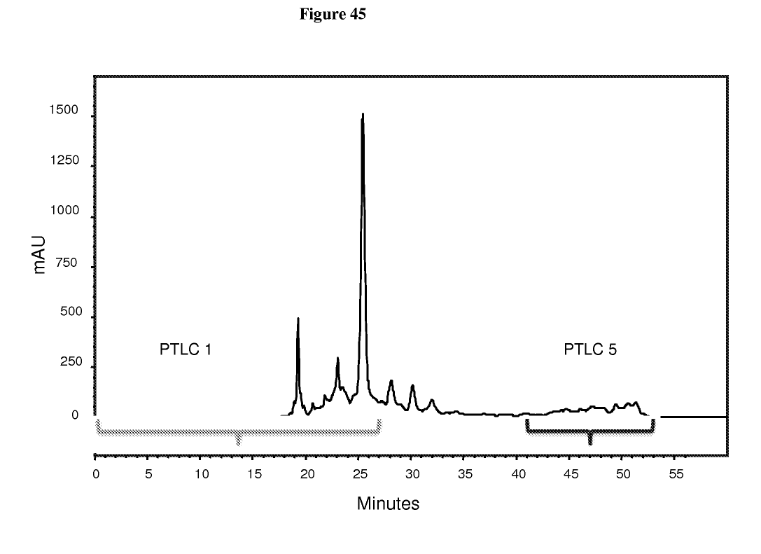

PTLC fractions and MPLC fractions were prepared using different methods, and have different compositions. G. buchananii stem bark extract were separated into PTLC1 and PTLC5 fractions using HPLC. Their chromatograms were compared (FIG. 45). Separations using HPLC showed that the main component of G. buchananii stem bark extract, which is in both cases the major peak in HPLC traces (shown here) and the main peak of MPLC separation (M3; manniflavanone) correspond with PTLC1 (orange bracket). By correlating the chromatograms and the biological effects of PTLC5 (blue bracket) correspond with M7.

Using prokinetic effect as marker, PTLC2 and to a smaller degree PTLC3 increased motility. This activity corresponds with M6. Therefore, these observations suggest that PTLC1 inhibited motility contains M4 (GB-2) and M5 (buchananiflavanone). Thus, PTLC1 seems to correspond with M1-M5.

Effect of Garcinia buchananii on Secretory Activity:

We used the Ussing chamber voltage clamp technique to measure the secretory activity of mucosal/submucosal preparations of guinea pig distal colon and human small and large intestinal specimen from patients undergoing abdominal surgery.

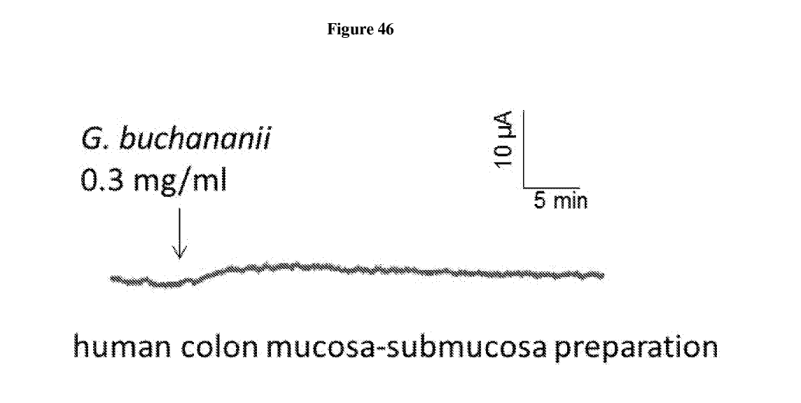

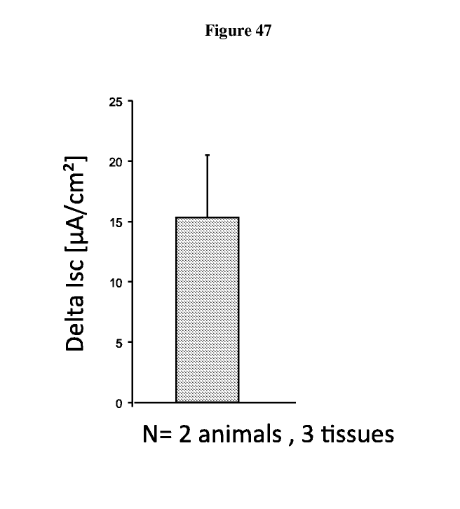

Preliminary studies showed that basolateral application of G. buchananii (0.15-0.6 mg/ml) induced a reproducible increase in the measured short circuit in human mucosal/submucosal preparations (n=4 patients, 6 tissues) (FIG. 46) as well as in the guinea pig preparations. Application of 0.3 mg/ml increased the measured short circuit current in guinea pig preparations by 15.3±5.2 μA/cm² (n=2 animals, 3 tissues) (FIG. 47). In the administered concentrations G. buchananii had no influence on nerve-mediated secretion. Secretory response to electrical field stimulation before and after application of G. buchananii was unchanged both in guinea pig and in human tissue preparations. Numerous experiments are needed to determine the concentration depended effects of G. buchananii extract and it's derivertive compounds in order to define the dose and compounds that cause reduced intestinal mucus and fluid secretion observed in vivo, in this case lactose-diarrhea treatment experiments.

Effect of Garcinia buchananii on Motility:

Organ bath experiments were done to measure the effect of G. buchananii on motility using circular muscle strip preparations of human small and large intestine from patients undergoing abdominal surgery. In the first experiments we used of G. buchananii in a concentration of 0.06 mg/ml, 0.12 mg/ml and 1.2 mg/ml (n=2 patients, 2 tissues), but we could detect no change in motility. In a concentration of 3.0 mg/ml in one preparation the tone was decreasing.

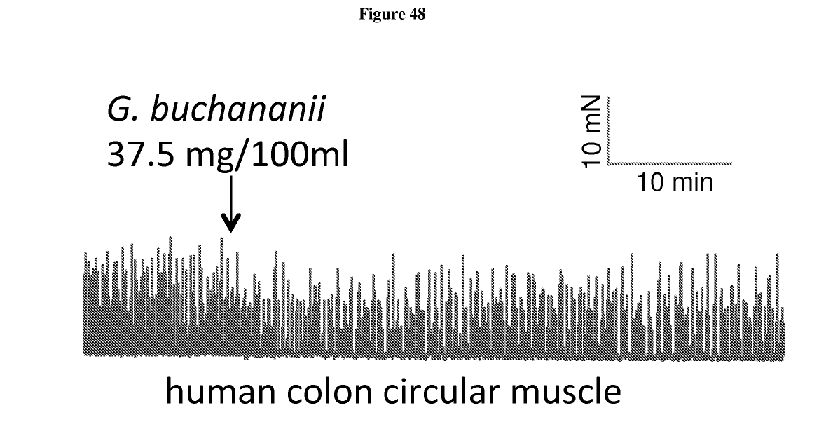

MPLC fraction 1 (M1) of G. buchananii [37.5 mg/100 ml] showed a spasmolytic effect by reducing amplitude and frequency of the contractions (FIG. 48). Thus, M1 showed an anti-motility effect. MPLC fraction 3 (M3) had a strong spasmolytic effect when used in a concentration of 102.5 mg/ml.

What is claimed is:

1. A composition comprising an isolated compound with a structure as defined in formula 11 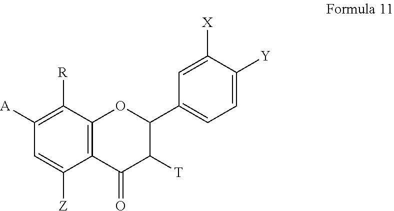
wherein A, X, Y and Z are each independently H, OH, halogen, CN, amine, imine or imide, and wherein R and T are bonds to other molecules with formula 11.

2. The composition of claim 1, wherein the isolated compound is a compound with a structure as defined in formula 5

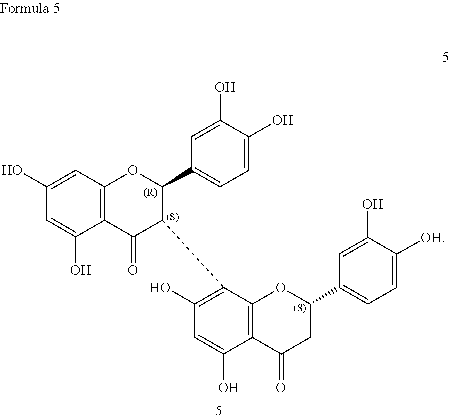

3. The composition of claim 1, wherein one of A, X, Y or Z must be halogen, CN, amine, imine or imide.

4. The composition of claim 1, wherein the composition is a pharmaceutical composition.

5. The composition of claim 4, further comprising a pharmaceutically acceptable salt and/or excipient.

6. The composition of claim 5, wherein the isolated compound has a formula is selected from the group consisting of

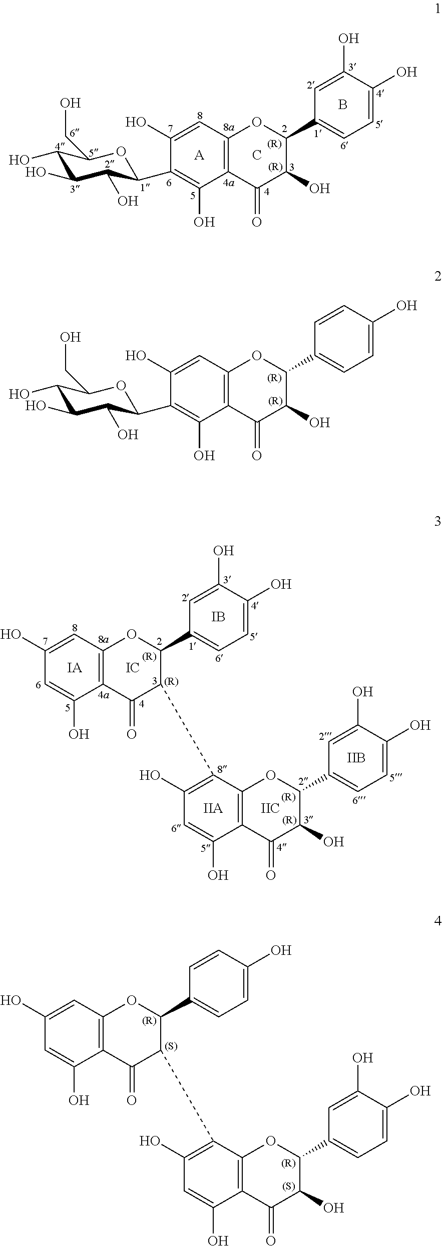

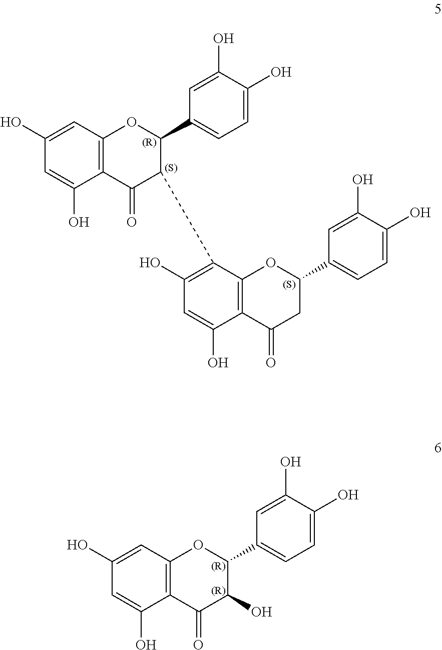

7. A composition comprising a fraction of an extract of a component of G. buchananii wherein the extract is enriched in a compound having a formula selected from the group consisting of

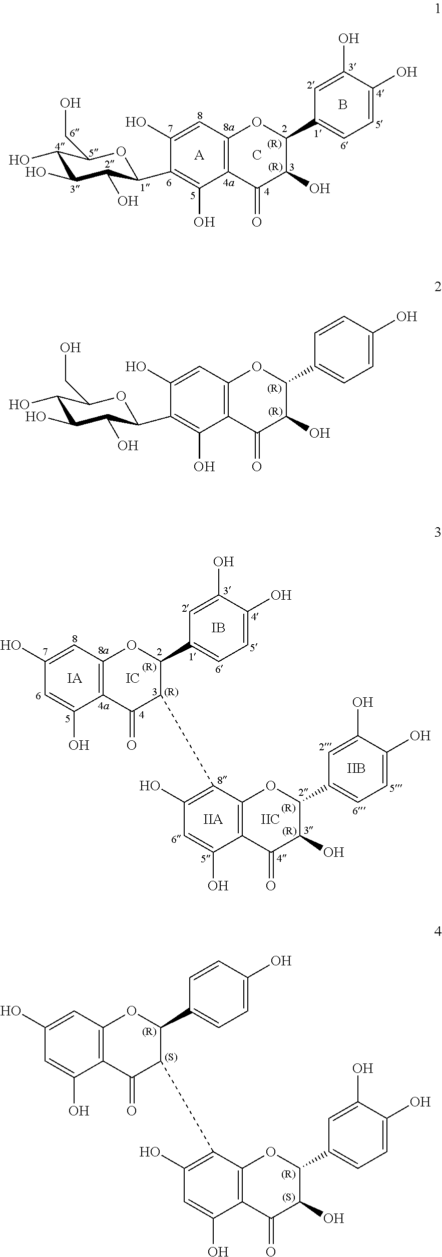

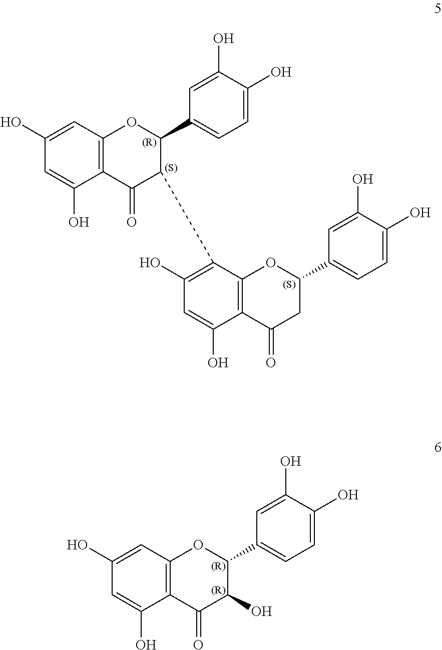

8. The composition of claim 7, wherein the component of G. buchananii is the bark of G. buchananii.

9. The composition of claim 8, wherein the bark is stem bark.

10. The composition of claim 7, wherein the fraction is isolated using preparative thin layer chromatography (PTLC).

11. The composition of claim 7, wherein the fraction is isolated using medium pressure liquid chromatography (MPLC) and high pressure liquid chromatography (HPLC).

12. A composition comprising a fraction of an extract of a component of G. buchananii wherein the extract is enriched in a compound with antioxidant properties.

13. The composition of claim 12, wherein the fraction has an EC₅₀ of less than 200 using the H₂O₂ assay.

14. The composition of claim 12, wherein the fraction has a value greater than 1 μmol TE/μmol) using the ORAC assay.

15. A method of inhibiting Ca²⁺ mobilization is smooth muscle cells by administering the composition of claim 1.

16. The method of claim 15, wherein the smooth muscle cells are in vivo.

17. A method of reducing the redox state of a biological system by administering the composition of claim 1.

18. The method of claim 17, wherein the biological system is selected from the group consisting of a cell, a tissue, an organ and an organism.

19. A method of treating diarrhea in a subject in need thereof comprising administering the composition of claim 1.

20. The method of claim 19, wherein the subject is a mammal.

21. A method of treating pain in a subject in need thereof comprising administering the composition of claim 1.

22. The method of claim 21, wherein the subject is a mammal.

23. A method of isolating the compound of claim 1 comprising:
1) producing an extract from G. buchananii bark comprising water and ethanol;
2) fractionating the extract using PTLC or MPLC or HPLC;
3) isolating fractions with antioxidant activity,
thereby isolating the compound of claim 1.

24. The method of claim 23, wherein the water is present in the extract at between 5 and 50%.

25. The method of claim 23, wherein the fractionation splits the extract into between 3 and 10 fractions.

26. The method of claim 25, wherein the fractionation splits the extract into between 5 and 8 fractions.

27. The method of claim 23, wherein the extract was filtered, extracted with hexane, evaporated and resuspended in a water/methanol mixture.

28. The method of claim 27, wherein the fractionation was performed by MPLC and HPLC.

29. The method of claim 23, wherein the fraction was performed by PTLC and HPLC.

30. A method of treating constipation in a subject in need thereof comprising administering a fraction of a component of G. buchananii wherein the fraction comprises at least one compound present in the PTLC2 or M6 fractions.

31. The method of claim 30, wherein the subject is a mammalian subject.

32. The method of claim 31, wherein the component of G. buchananii is the bark of G. buchananii.

33. The method of claim 32, wherein the bark is stem bark.

34. The method of claim 30, wherein the fraction is isolated using preparative thin layer chromatography (PTLC).

35. The method of claim 30, wherein the fraction is isolated using medium pressure liquid chromatography (MPLC).

36. The method of claim 30, wherein the fraction is isolated using or high pressure liquid chromatography (HPLC).
